(19) United States
(12) Patent Application Publication
(10) Pub. No.: US 2021/0113656 A1
(43) Pub. Date: Apr. 22, 2021

(54) TREATMENT OF STAGE III NSCLC AND MITIGATION OF PATHOLOGICAL CONDITIONS ASSOCIATED WITH THE TREATMENT

(71) Applicant: Merck Patent GmbH, Darmstadt (DE)

(72) Inventors: Italia Grenga, Burlington, MA (US); Isabelle Dussault, Needham, MA (US); Yulia Vugmeyster, Winchester, MA (US); Akash Khandelwal, Griesheim (DE); Olaf Christensen, Cambridge, MA (US); Samer El Bawab, Frankfurt Am Main (DE); Yan Lan, Belmont, MA (US)

(21) Appl. No.: 17/117,485

(22) Filed: Dec. 10, 2020

Related U.S. Application Data
(63) Continuation of application No. PCT/US2019/036725, filed on Jun. 12, 2019.
(60) Provisional application No. 62/855,170, filed on May 31, 2019.
(60) Provisional application No. 62/800,808, filed on Feb. 4, 2019.
(60) Provisional application No. 62/684,385, filed on Jun. 13, 2018.

(51) Int. Cl. A61K 38/17; C07K 16/28; A61K 51/00; A61P 35/00; A61K 33/243; A61K 31/7048; A61K 31/519; A61K 31/337

(52) U.S. Cl. CPC A61K 38/179; C07K 16/2827; A61K 51/00; A61P 35/00; A61K 2039/505; A61K 31/7048; A61K 31/519; A61K 31/337; C07K 2319/33; A61K 33/243

(57) ABSTRACT

This disclosure relates generally to dosage regimens for targeted TGF-β inhibition with a bi-functional fusion protein for use in a method of treating a treatment naïve patient diagnosed with stage III non-small cell lung cancer (NSCLC), and/or mitigating a pathological condition associated with chemotherapy and radiotherapy (cCRT).

### CROSS-REFERENCE TO RELATED APPLICATIONS

This application claims the benefit of and priority to U.S. Provisional Patent Application No. 62/684,385, filed Jun. 13, 2018; to U.S. Provisional Patent Application No. 62/800,808, filed Feb. 4, 2019; and to U.S. Provisional Patent Application No. 62/855,170, filed May 31, 2019, the entire disclosures of which are incorporated by reference herein.

### SEQUENCE LISTING

The instant application contains a Sequence Listing which has been submitted electronically in ASCII format and is hereby incorporated by reference in its entirety. Said ASCII copy, created on May 31, 2019, is named EMD-010WO_SL_ST25.txt and is 75,888 bytes in size.

### FIELD OF THE DISCLOSURE

The present disclosure relates generally to dosage regimens for targeted TGF-β inhibition with a bi-functional fusion protein for use in a method of treating a treatment naïve subject diagnosed with stage III non-small-cell lung cancer (NSCLC), and/or mitigating a pathological condition associated with chemotherapy and radiotherapy (cCRT).

### BACKGROUND

Treatment of locally advanced, unresectable, stage III NSCLC with chemotherapy and concurrent radiation therapy (cCRT) often fails to contain disease progression in NSCLC patients. Moreover, radiation therapy causes pathological conditions, e.g., pulmonary fibrosis. Radiation-induced fibrosis of the lung may occur in lung tissue irradiated at ≥20 Gy within the first 6 months after initiation of treatment.

TGFβ is a major profibrotic molecule that contributes to the development of pulmonary fibrosis. US patent application publication number US 20150225483 A1, incorporated herein by reference, describes a bi-functional fusion protein that combines an anti-programmed death ligand 1 (PD-L) antibody with the soluble extracellular domain of tumor growth factor beta receptor type II (TGFβRII) as a TGFβ neutralizing “Trap,” into a single molecule. Specifically, the protein is a heterotetramer, consisting of the two immunoglobulin light chains of anti-PD-L1, and two heavy chains comprising the heavy chain of anti-PD-L genetically fused via a flexible glycine-serine linker to the extracellular domain of the human TGFβRII (see FIG. 1). This anti-PD-L1/TGFβ Trap molecule is designed to target two major mechanisms of immunosuppression in the tumor microenvironment. US patent application publication number US 20150225483 A1 describes administration of the Trap molecule at doses based on the patient's weight.

The present disclosure provides dosage regimens for targeted TGF-β inhibition with an anti-PD-L1/TGFβ Trap molecule for use in a method of treating a treatment naïve subject diagnosed with stage III NSCLC, and/or mitigating pathological conditions (e.g., pulmonary fibrosis, pneumonitis) associated with concurrent cCRT.

### SUMMARY OF THE DISCLOSURE

For an effective treatment of patients diagnosed with stage III NSCLC, and to counter acute and long term symptomatic lung injury due to fibrosis, the present disclosure provides a therapeutic regimen that treats stage III NSCLC, and spares as much normal lung tissue as possible from radiation-induced damage, and, thereby improves disease prognosis and overall survival of the NSCLC patients.

In one aspect, the present disclosure provides an anti-PD-L1/TGFβ Trap with concomitant cCRT to simultaneously target two immune suppressive pathways: PD-L1 and TGF-β, and, thereby treat stage III NSCLC, while minimizing the development of pathological conditions (e.g., pulmonary fibrosis, pneumonitis) associated with concomitant radiotherapy, and increasing the time-to-onset of metastasis and/or time to distant metastasis of the stage III NSCLC in the patient.

The present disclosure provides improved dosing regimens for administration of bifunctional proteins targeting PD-L1 and TGFβ for treating stage III NSCLC, while minimizing the development of pathological conditions (e.g., pulmonary fibrosis, pneumonitis) associated with concomitant radiotherapy, and increasing the time-to-onset of metastasis and/or time to distant metastasis of the stage III NSCLC in the patient. Specifically, body weight independent (BW-independent) dosing regimens and related dosage forms involving administration of at least 500 mg (e.g., 1200 mg, 1800 mg, 2400 mg) of the bifunctional protein administered at various dosing frequencies can be used as an anti-tumor and anti-cancer therapeutic for treating stage III NSCLC, while minimizing the development of pathological conditions (e.g., pulmonary fibrosis, pneumonitis) associated with concomitant radiotherapy, and increasing the time-to-onset of metastasis and/or time to distant metastasis of the stage III NSCLC in the patient. The BW-independent dosing regimen ensures that all stage III NSCLC patients, irrespective of their body weight, will have adequate drug exposure at the tumor site.

The bifunctional protein of the present disclosure (anti-PD-L1/TGFβ Trap molecule) includes a first and a second polypeptide. The first polypeptide includes: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor (TGFβ) (e.g., a soluble fragment). The second polypeptide includes at least a variable region of a light chain of an antibody that binds PD-L1, in which the heavy chain of the first polypeptide and the light chain of the second polypeptide, when combined, form an antigen binding site that binds PD-L1 (e.g., any of the antibodies or antibody fragments described herein). Because the bifunctional protein of the present disclosure binds to two targets, (1) PD-L1, which is largely membrane bound, and (2) TGFβ, which is soluble in blood and interstitium, the BW-independent dosing regimen requires a dose that is effective not only to inhibit PD-L1 at the tumor site but also sufficient to inhibit TGFβ.

In one aspect, the disclosure provides dosage regimens for targeted TGF-β inhibition with a bi-functional fusion protein for use in a method of treating a treatment naïve subject diagnosed with stage III non-small cell lung cancer (NSCLC), and/or mitigating a pathological condition associated with chemotherapy and radiotherapy (cCRT).

In one aspect, the disclosure provides dosage regimens for targeted TGF-β inhibition with a bi-functional fusion protein for use in a method of treating a stage III NSCLC that exhibits squamous or non-squamous histology, and/or mitigating a pathological condition associated with chemotherapy and radiotherapy (cCRT). In certain embodiments, the stage III NSCLS is unresectable.

In one aspect, the present disclosure provides a method of treating advanced unresectable stage III NSCLC in a patient by administering to the patient an anti-PD-L/TGFβ Trap of the present disclosure in combination with cCRT (e.g., platinum-based chemoradiation), followed by administering the anti-PD-L1/TGFβ Trap to the patient. In certain embodiments, the present disclosure provides a method of treating advanced unresectable stage III NSCLC in a patient by administering to the patient an anti-PD-L1/TGFβ Trap in combination with and following concurrent platinum-based chemoradiation.

In certain embodiments, cCRT is administered as either cisplatin/etoposide, cisplatin/pemetrexed, or carboplatin/paclitaxcel concurrently with radiation (e.g., radiation delivered by intensity-modulated radiation therapy).

In certain embodiments, the present disclosure provides a method of treating advanced unresectable stage III NSCLC, which has a non-squamous histology, in a patient by administering to the patient an anti-PD-L1/TGFβ Trap in combination with cCRT (e.g., cisplatin/pemetrexed and radiation) followed by administering the anti-PD-L1/TGFβ Trap to the patient. In certain embodiments, the present disclosure provides a method of treating advanced unresectable stage III NSCLC in a patient by administering to the patient an anti-PD-L1/TGFβ Trap in combination with and following concurrent cisplatin/pemetrexed and radiation (e.g., radiation delivered by intensity-modulated radiation therapy).

The disclosure also features a method of promoting local depletion of TGFβ. The method includes administering a protein described above, where the protein binds TGFβ in solution, binds PD-L1 on a cell surface, and carries the bound TGFβ into the cell (e.g., a cancer cell).

The disclosure also features a method of inhibiting SMAD3 phosphorylation in a cell (e.g., a cancer cell or an immune cell), the method including exposing the cell in the tumor microenvironment to a protein described above.

Other embodiments and details of the disclosure are presented herein below.

### BRIEF DESCRIPTION OF THE DRAWINGS

FIG. 1 is a schematic drawing of an anti-PD-L1/TGFβ Trap molecule including one anti-PD-L1 antibody fused to two extracellular domains (ECDs) of TGFβ Receptor II via a (Gly₄Ser)₄Gly (SEQ ID NO: 11) linker.

FIG. 2 shows a graph of a two-step ELSA demonstrating that anti-PD-L1/TGFβ Trap simultaneously binds to both PD-L1 and TGFβ.

FIG. 3 is a graph showing anti-PD-L1/TGFβ Trap induces a dramatic increase in IL-2 levels.

FIG. 4A is a graph showing in vivo depletion of TGFβ1 in response to the anti-PD-L1/TGFβ Trap. Line graphs represent naïve, isotype control, and three different doses, as indicated in the legend. FIG. 4B is a graph showing in vivo depletion of TGFβ2 in response to the anti-PD-L1/TGFβ Trap. Line graphs represent naïve, isotype control, and three different doses, as indicated in the legend. FIG. 4C is a graph showing in vivo depletion of TGFβ3 in response to the anti-PD-L1/TGFβ Trap. Line graphs represent naïve, isotype control, and three different doses, as indicated in the legend. FIG. 4D is a graph showing that occupancy of PD-L by the anti-PD-L1/TGFβ Trap supports a receptor binding model in the EMT-6 tumor system.

FIG. 5 is a graph showing anti-tumor efficacy of anti-PD-L1/TGFβ Trap control (anti-PD-L1(mut)/TGFβ) in Detroit 562 xenograft model.

FIG. 6A is a box-plot of C_{avg} distribution for an entire population for a fixed (1200 mg) versus mg/kg based dosing (17.65 mg/kg) in a simulated population of 68 kg median body weight. FIG. 6B is a box-plot of exposure AUC distribution for an entire population for a fixed (1200 mg) versus mg/kg based dosing (17.65 mg/kg) in a simulated population of 68 kg median body weight. FIG. 6C is a box-plot of C_{trough} distribution for an entire population for a fixed (1200 mg) versus mg/kg based dosing (17.65 mg/kg) in a simulated population of 68 kg median body weight. FIG. 6D is a box-plot of Cₘₐₓ distribution for an entire population for a fixed (1200 mg) versus mg/kg based dosing (17.65 mg/kg) in a simulated population of 68 kg median body weight.

FIG. 6E is a box-plot of C_{avg} distribution for an entire population for a fixed (500 mg) versus mg/kg based dosing (7.35 mg/kg) in a simulated population of 68 kg median body weight. FIG. 6F is a box-plot of exposure AUC distribution for an entire population for a fixed (500 mg) versus mg/kg based dosing (7.35 mg/kg) in a simulated population of 68 kg median body weight. FIG. 6G is a box-plot of C_{trough} distribution for an entire population for a fixed (500 mg) versus mg/kg based dosing (7.35 mg/kg) in a simulated population of 68 kg median body weight. FIG. 6H is a box-plot of Cₘₐₓ distribution for an entire population for a fixed (500 mg) versus mg/kg based dosing (7.35 mg/kg) in a simulated population of 68 kg median body weight.

FIGS. 7A-7C are graphs showing the predicted PK and PD-L1 receptor occupancy (“RO”) of anti-PD-L1/TGFβ Trap molecules at doses and schedules associated with tumor stasis in mice. FIG. 7A is a graph showing the predicted plasma concentration vs. time. FIG. 7B is a graph showing the predicted PD-L1 RO vs. time in PBMC. FIG. 7C is a graph showing the predicted PD-L1 RO vs. time in tumor.

FIG. 8 represents box plots of gene expression signatures associated fibrosis in control mice (untreated), and in mice treated with an anti-PD-L1/TGFβ Trap molecule, radiation, and anti-PD-L1/TGFβ Trap molecule and radiation.

FIG. 9 represents gene expression signatures of Cxcl12, Fap, and Cdc6 (based on RNA sequencing analysis) after mice were treated with radiation, anti-PD-L1/TGFβ Trap molecule, and concomitant anti-PD-L1/TGFβ Trap and radiation. “Control” represents gene expression in mice that remained untreated.

FIG. 10 is a schematic diagram of the therapeutic regimen described in Example 3. Stable disease, partial response, and complete response are denoted by SD, PR, and CR, respectively.

FIG. 11 is a schematic diagram of the therapeutic regimen described in Example 4. Stable disease, partial response, and complete response are denoted by SD, PR, and CR, respectively.

FIGS. 12A-12C are bar graphs showing that anti-PD-L1/TGFβ Trap and Trap control, but not anti-PD-L1 decrease chemotherapy-induced fibrosis. FIG. 12A shows that while anti-PD-L1 antibody did not affect the collagen content relative to isotype control, both Trap control and anti-PD-L1/TGFβ Trap treatment significantly decreased collagen content (total collagen (percent picrosirius red (PSR); PSR staining is a commonly used histological technique to visualize collagen in paraffin-embedded tissue sections. PSR stained collagen appears red in light microscopy)); p=0.0038 and p=0.0019, respectively). FIG. 12B shows that while anti-PD-L1 antibody did not affect the percent αSMA relative to isotype control, both Trap control and anti-PD-L1/TGFβ Trap treatment significantly decreased the percent αSMA (p=0.0003 and p=0.0013, respectively). FIG. 12C are bar graphs showing that anti-PD-L1/TGFβ Trap reduces the ratio of pSmad2/3 relative to isotype control treatment (p=0.0006).

FIG. 13A is a scatterplot showing that anti-PD-L1/TGFβ Trap monotherapy resulted in a reduction in the epithelial-mesenchymal transition (EMT) signature score relative to isotype control (p<0.0001), and that the combination of anti-PD-L1/TGFβ Trap and radiation therapy significantly downregulated the EMT signature score relative to isotype control (p<0.0001).

FIG. 13B is a scatterplot showing that pro-fibrotic gene signature scores were also decreased by anti-PD-L1/TGFβ Trap monotherapy but were significantly increased by radiation therapy relative to isotype control (p<0.0001). Furthermore, combining radiation with anti-PD-L1/TGFβ Trap reduced pro-fibrotic signature score relative to radiation alone.

FIG. 14A depicts box-plots showing that anti-PD-L1/TGF$ Trap combined with radiation therapy significantly reduced ACTA2 expression. While radiation treatment alone had no significant effect on ACTA2 expression, anti-PD-L1/TGFβ Trap monotherapy and anti-PD-L1/TGFβ Trap combined with radiation therapy significantly reduced ACTA2 expression in the 4T1 model (p<0.0001 and p=0.0236, respectively).

FIG. 14B depicts box-plots showing that anti-PD-L1/TGFβ Trap significantly reduced CTGF expression relative to isotype control (p=0.0019) and, while radiation treatment increased CTGF, as expected, anti-PD-L1/TGFβ Trap combination significantly counteracted the effects of radiation treatment compared to radiation monotherapy (P=0.0024).

FIG. 14C depicts box-plots showing that anti-PD-L1/TGFβ Trap significantly reduced FAP expression relative to isotype control (p<0.0001) and the reduction in FAP seen with radiation therapy was further reduced by the combination of anti-PD-L1/TGFβ Trap with radiation (P=0.0054).

FIG. 15 depicts box-plots showing the number of α-SMA+ pixels determined for multiple regions of interest (ROIs) per tumor and normalized to ROI area; each symbol represents the proportion of positive pixels for a single tumor. P-values were determined by one-way ANOVA. Scale bars, 250 m.

FIGS. 16A-16D are images showing that anti-PD-L1/TGFβ Trap treatment reduces α-SMA expression in mouse tumors. Relative to isotype control (FIG. 16A), anti-PD-L1/TGFβ Trap treatment significantly reduced α-SMA expression (p<0.0001) (FIG. 16B), while radiation therapy significantly increased α-SMA expression (p=0.0002) (FIG. 16C). The combination of anti-PD-L1/TGFβ Trap with radiation therapy significantly reduced α-SMA expression relative to radiation monotherapy (p=0.0001) (FIG. 16D), suggesting that anti-PD-L1/TGFβ Trap can reduce radiation-induced cancer-associated fibroblasts (CAFs) activity.

FIG. 17 is a schematic diagram of the therapeutic regimen described in Example 6. Stable disease, partial response, and complete response are denoted by SD, PR, and CR, respectively.

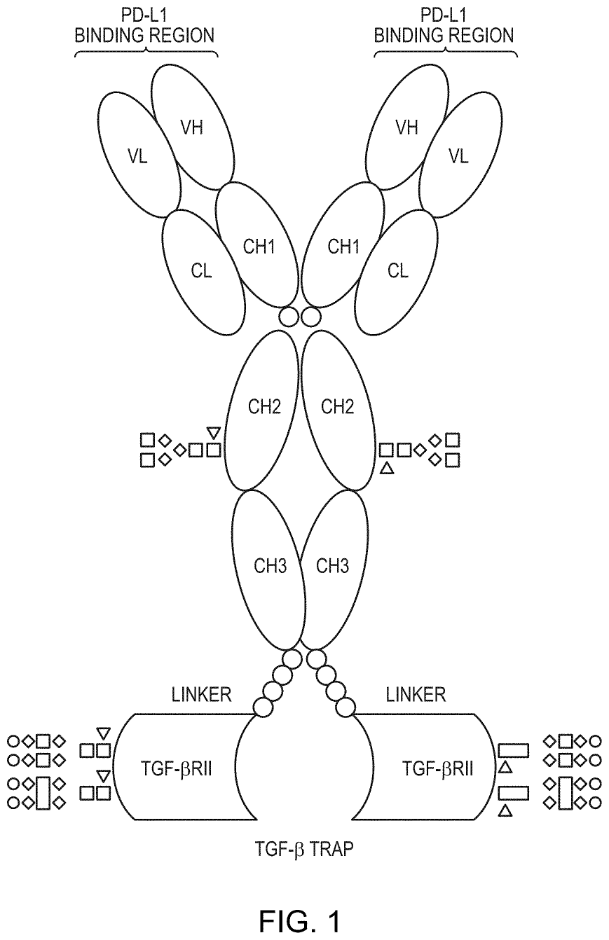

### DETAILED DESCRIPTION

By “TGFβRII” or “TGFβ Receptor II” is meant a polypeptide having the wild-type human TGFβ Receptor Type 2 Isoform A sequence (e.g., the amino acid sequence of NCBI Reference Sequence (RefSeq) Accession No. NP_001020018 (SEQ ID NO: 8)), or a polypeptide having the wild-type human TGFβ Receptor Type 2 Isoform B sequence (e.g., the amino acid sequence of NCBI RefSeq Accession No. NP_003233 (SEQ ID NO: 9)) or having a sequence substantially identical to the amino acid sequence of SEQ ID NO: 8 or of SEQ ID NO: 9. The TGFβRII may retain at least 0.1%, 0.5%, 1%, 5%, 10%, 25%, 35%, 50%, 75%, 90%, 95%, or 99% of the TGFβ-binding activity of the wild-type sequence. The polypeptide of expressed TGFβRII lacks the signal sequence.

By a “fragment of TGFβRII capable of binding TGFβ” is meant any portion of NCBI RefSeq Accession No. NP_001020018 (SEQ ID NO: 8) or of NCBI RefSeq Accession No. NP_003233 (SEQ ID NO: 9), or a sequence substantially identical to SEQ ID NO: 8 or SEQ ID NO: 9 that is at least 20 (e.g., at least 30, 40, 50, 60, 70, 80, 90, 100, 110, 120, 130, 140, 150, 160, 175, or 200) amino acids in length that retains at least some of the TGFβ-binding activity (e.g., at least 0.1%, 0.5%, 1%, 5%, 10%, 25%, 35%, 50%, 75%, 90%, 95%, or 99%) of the wild-type receptor or of the corresponding wild-type fragment. Typically such fragment is a soluble fragment. An exemplary such fragment is a TGFβRII extra-cellular domain having the sequence of SEQ ID NO: 10.

“Treatment naïve” refers to subjects or patients who have not received prior systemic treatment for their stage III NSCLC since being diagnosed with the disease. In various embodiments of the present disclosure, treatment naïve patients have not received prior therapy with an anti-PD-1, anti-PD-L, or anti-Cytotoxic T-lymphocyte-associated antigen-4 (CTLA-4) antibody (including ipilimumab), or any other antibody or drug specifically targeting T-cell co-stimulation or checkpoint pathways. In various embodiments of the present disclosure, treatment naïve patients are selected for first-line (1L) treatment of the present invention.

“PD-L1 positive” or “PD-L1+” indicates ≥1% PD-L1 positive tumor cells as determined, for example, by the Dako IHC 22C3 PharmDx assay, or by the VENTANA PD-L1 (SP263) assay.

“PD-L1 high” or “high PD-L1” refers to ≥80% PD-L positive tumor cells as determined by the PD-L1 IHC 73-10 assay (Dako), or tumor proportion score (TPS)≥50% as determined by the Dako IHC 22C3 PharmDx assay (TPS is a term of art related to the IHC 22C3 PharmDx assay, which describes the percentage of viable tumor cells with partial or complete membrane staining (e.g., staining for PD-L1)). Both IHC 73-10 and IHC 22C3 assays select a similar patient population at their respective cutoffs. In certain embodiments, VENTANA PD-L1 (SP263) assay, which has high concordance with 22C3 PharmDx assay (see Sughayer et al., Appl. Immunohistochem. Mol. Morphol., (2018)), can also be used for determining PD-L1 high expression level.

By “substantially identical” is meant a polypeptide exhibiting at least 50%, desirably 60%, 70%, 75%, or 80%, more desirably 85%, 90%, or 95%, and most desirably 99% amino acid sequence identity to a reference amino acid sequence. The length of comparison sequences will generally be at least 10 amino acids, desirably at least 15 contiguous amino acids, more desirably at least 20, 25, 50, 75, 90, 100, 150, 200, 250, 300, or 350 contiguous amino acids, and most desirably the full-length amino acid sequence.

By “patient” is meant either a human or non-human animal (e.g., a mammal). “Patient,” “subject,” “patient in need thereof,” and “subject in need thereof” are used interchangeably in the present disclosure, and refer to a living organism suffering from or prone to a disease or condition that can be treated by administration using the methods and compositions provided in the present disclosure.

The terms “treat,” “treating,” or “treatment,” and other grammatical equivalents as used in the present disclosure, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

The term “consolidation” in the context of a therapeutic regimen of the present disclosure is used as is commonly understood in the art. For example, according to the National Cancer Institute, the term “consolidation therapy” is a “[t]reatment that is given after cancer has disappeared following the initial therapy. Consolidation therapy is used to kill any cancer cells that may be left in the body. It may include radiation therapy, a stem cell transplant, or treatment with drugs that kill cancer cells. Also called intensification therapy and postremission therapy.” https://www.cancer.gov/publications/dictionaries/cancer-terms/def/consolidation-therapy, last visited on Jun. 9, 2018.

The term “progression-free survival” or PFS is defined as the time from randomization (which can occur 6 or more weeks after treatment initiation) to the date of the first documented event of tumor progression or death in the absence of disease progression. The term “overall survival” is defined as the time from randomization until death from any cause. Progression-free survival is assessed by the investigators, according to RECIST, version 1.1, as a predefined sensitivity analysis.

The term “mitigate,” “mitigating,” or “mitigation,” and other grammatical equivalents as used in the present disclosure, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis.

By “cancer” is meant stage III (stage IIIA, stage IIIB and/or stage IIIC) non-small cell lung cancer (NSCLC) is used according to its plain and ordinary meaning, characterized by, for example, by the National Cancer Institute of the United States of America. Thus, in various embodiments, the cancer has spread, for example, to lymph nodes on the same side of the primary tumor or to lymph nodes on the opposite side of the chest as the primary tumor.

The term ‘unresectable’ means a cancer that cannot be removed through surgery.

The terms “risk,” “at risk,” and “risk factor,” are used here as conventionally understood in the art. For example, a risk factor is any attribute, characteristic or exposure of an individual that increases the likelihood of developing a disease or injury. In certain embodiments, a person at risk of developing a disease, disorder, or condition means that the person is exposed to a risk factor that contributes or enhances the probability of incidence of that disease, disorder, or condition.

Throughout the description and claims of the present disclosure the word “comprise” and other forms of the word, such as “comprising” and “comprises,” means including but not limited to, and is not intended to exclude, for example, other components.

By “co-administer” it is meant that a composition described herein is administered at the same time, just prior to, or just after the administration of additional therapies. The protein and the composition of the present disclosure can be administered alone or can be co-administered with a second, third, or fourth therapeutic agent(s) to a patient. Co-administration is meant to include simultaneous or sequential administration of the protein or composition individually or in combination (more than one therapeutic agent).

The term “a” is not meant to limit as a singular. In certain embodiments, the term “a” may refer to a plural form. As used throughout the present disclosure, the singular forms “a,” “an,” and “the” include plural reference unless the context clearly dictates otherwise. Thus, for example, a reference to “a composition” includes a plurality of such compositions, as well as a single composition.

A “reconstituted” formulation is one which has been prepared by dissolving a lyophilized formulation in an aqueous carrier such that the bifunctional molecule is dissolved in the reconstituted formulation. The reconstituted formulation is suitable for intravenous administration (IV) to a patient in need thereof.

The term “about” refers to any minimal alteration in the concentration or amount of an agent that does not change the efficacy of the agent in preparation of a formulation and in treatment of a disease or disorder. In embodiments, the term “about” may include ±15% of a specified numerical value or data point.

Ranges can be expressed in the present disclosure as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed in the present disclosure, and that each value is also disclosed as “about” that particular value in addition to the value itself. It is also understood that throughout the application, data are provided in a number of different formats and that the data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

An “isotonic” formulation is one which has essentially the same osmotic pressure as human blood. Isotonic formulations will generally have an osmotic pressure from about 250 to 350 mOsmol/kgH₂O. The term “hypertonic” is used to describe a formulation with an osmotic pressure above that of human blood. Isotonicity can be measured using a vapor pressure or ice-freezing type osmometer, for example.

The term “buffering agent” refers to one or more components that when added to an aqueous solution is able to protect the solution against variations in pH when adding acid or alkali, or upon dilution with a solvent. In addition to phosphate buffers, there can be used glycinate, carbonate, citrate buffers and the like, in which case, sodium, potassium or ammonium ions can serve as counterion.

An “acid” is a substance that yields hydrogen ions in aqueous solution. A “pharmaceutically acceptable acid” includes inorganic and organic acids which are nontoxic at the concentration and manner in which they are formulated.

A “base” is a substance that yields hydroxyl ions in aqueous solution. “Pharmaceutically acceptable bases” include inorganic and organic bases which are non-toxic at the concentration and manner in which they are formulated.

A “lyoprotectant” is a molecule which, when combined with a protein of interest, prevents or reduces chemical and/or physical instability of the protein upon lyophilization and subsequent storage.

A “preservative” is an agent that reduces bacterial action and may be optionally added to the formulations herein. The addition of a preservative may, for example, facilitate the production of a multi-use (multiple-dose) formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3pentanol, and m-cresol.

A “surfactant” is a surface active molecule containing both a hydrophobic portion (e.g., alkyl chain) and a hydrophilic portion (e.g., carboxyl and carboxylate groups). Surfactant may be added to the formulations of the invention. Surfactants suitable for use in the formulations of the present invention include, but are not limited to, polysorbates (e.g. polysorbates 20 or 80); poloxamers (e.g. poloxamer 188); sorbitan esters and derivatives; Triton; sodium laurel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linolcamidopropyl-, myristamidopropyl-, palmidopropyl-, or isostearamidopropylbetaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethylene glycol, polypropyl glycol, and copolymers of ethylene and propylene glycol (e.g., Pluronics, PF68 etc.).

### Body Weight-Independent Dosing Regimen

Body weight-independent dosing regimens involving the administration to treatment naïve patients of at least 500 mg of the bifunctional anti-PD-L1/TGFβ Trap molecules described herein have been developed, informed by the results of a variety of pre-clinical and clinical assessments of the molecules. Two studies investigated the safety, tolerability, and pharmacokinetics of the molecules, and included assessments of PD-L1 target occupancy on peripheral blood mononuclear cells obtained from the blood of treated patients and measurements of the concentrations of TGFβ1, TGFβ2, and TGFβ3. These assessments were based on data from a total of 350 subjects (dose escalation cohorts of 1, 3, 10 and 20 mg/kg in solid tumors, and expansion cohorts of 3 mg/kg, 10 mg/kg, 500 mg, and 1200 mg in selected tumor types).

### PK/Efficacy Model (Mouse Model)

Experiments were also conducted to determine the efficacy of the anti-PD-L1/TGFβ Trap molecule in a tumor model. Efficacy results from EMT-6 xenografts were used to establish the PK/Efficacy model. The established PK model in mice was used to simulate anti-PD-L1/TGFβ Trap plasma exposure for the efficacy experiment settings. The estimated parameters are reported in Table 1. The estimated KC50 value was 55.3 μg/mL, which represents the average plasma concentrations for which 50% of the maximal anti-tumor activity of the anti-PD-L1/TGFβ Trap molecule could be achieved.

Basic diagnostics plots of the model revealed no model misspecification. The model predictions are able to capture the tumor volume distributions. Conditional weighted residuals are normally distributed with a 0 mean and 1 variance without a trend. The PK/Efficacy model was then used to simulate tumor growth inhibition (TGI) using the human predicted concentration-time profiles at different doses.

TABLE 1
Mouse PK/Efficacy model parameters for anti-PD-
L1/TGFβ Trap molecule in EMT-6 xenograft mice

| Parameters | Estimate | Std | CV % | % IIV |
|---|---|---|---|---|
| K_{g} (h⁻¹) | 0.068 | 0.0005 | 0.82 | 40 |
| Kₕ (h⁻¹) | 0.055 | 0.0024 | 4.4 | 76 |
| KC₅₀ (ng/mL) | 55324.6 | 522.3 | 4.4 | 232 |
| Kₘₐₓ | 2 | 0.09 | 1 | 93 |
| Baseline (mm³) | 88.3 | 0.87 | 1 | 47 |

### Response Analysis Based on PD-L1 Occupancy (in a Mouse Model)

Using the efficacy experiments, responses in mice have been analyzed and sorted by either tumor regression or tumor stasis, and PK and PD-L1 receptor occupancy (RO) have been predicted based on the integrated PK/RO model. The approach demonstrated that an anti-PD-L1/TGFβ Trap molecule plasma concentration between 40 and 100 μg/mL associated with a PD-L1 RO above 95% in tumor is required to reach tumor regression. The plasma concentration of anti-PD-L1/TGFβ Trap molecule between 10 and 40 μg/mL associated with a PD-L1 RO above 95% in periphery is required to reach tumor stasis.

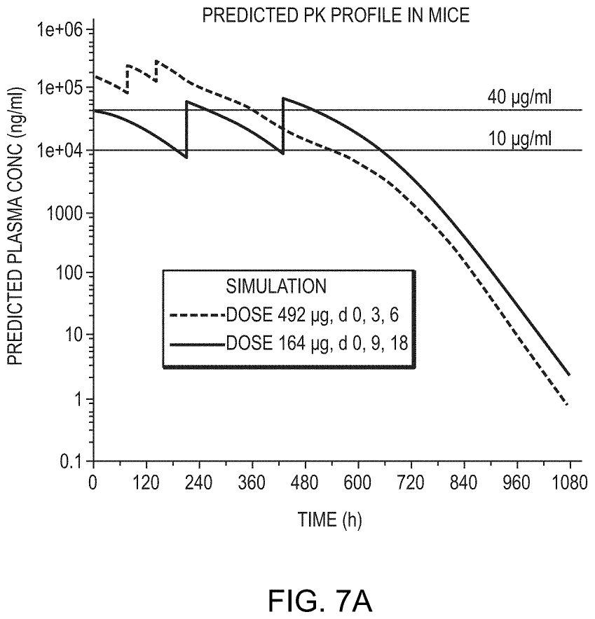

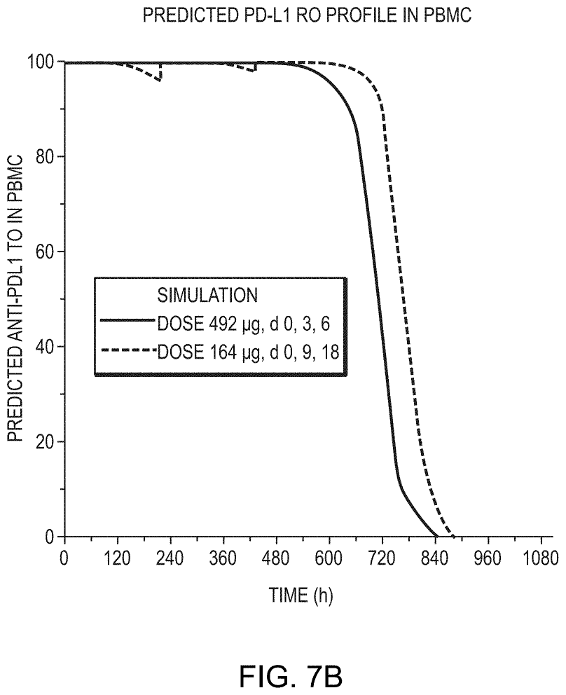

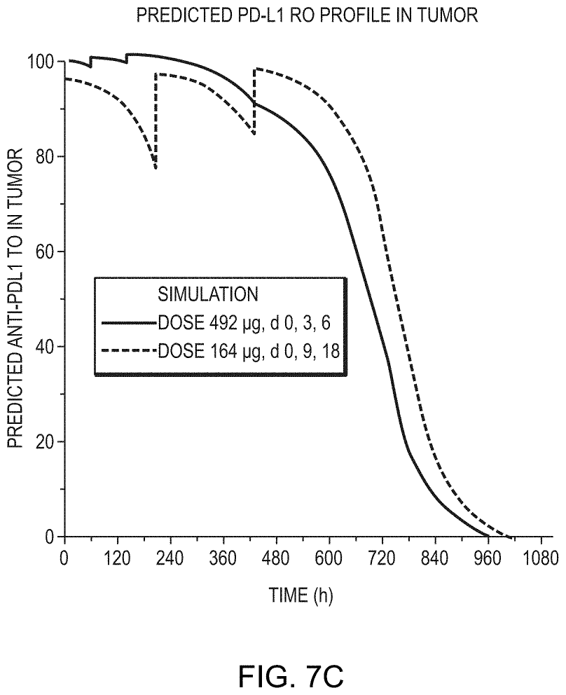

Response analysis and predicted PK/RO in mice lead to FIGS. 7A-7C, which summarize the PK/RO/Efficacy for the anti-PD-L1/TGFβ Trap molecule in mice. 95% of PD-L1 RO is achieved at a plasma concentration of 40 μg/mL with an expected/estimate TGI of only about 65%. Increasing the concentration above 40 μg/mL results in an additional increase in tumor growth inhibition. 95% of tumor growth inhibition is achieved at average plasma concentration of about 100 μg/mL.

Based on the population PK model described below, a flat dose of at least 500 mg administered once every two weeks is required to maintain an average concentration of about 100 μg/mL, while a flat dose of about 1200 mg administered once every two weeks is required to maintain a C_{trough} of about 100 μg/mL. In certain embodiments about 1200 mg to about 3000 mg (e.g., about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg about 2400 mg, etc.) of a protein product of the present disclosure (e.g., anti-PD-L1/TGFβ Trap) is administered to a subject. In certain embodiments, about 1200 mg of anti-PD-L1/TGFβ Trap molecule is administered to a subject once every two weeks. In certain embodiments, about 1800 mg of anti-PD-L1/TGFβ Trap molecule is administered to a subject once every three weeks.

In embodiments, about 1200 mg to about 3000 mg (e.g., about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, etc.) of the protein product with a first polypeptide that includes the amino acid sequence of SEQ ID NO: 3, and a second polypeptide that includes the amino acid sequence of SEQ ID NO: 1 is administered to a subject. In certain embodiments, about 1200 mg to about 3000 mg (e.g., about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, etc.) of the protein product with a first polypeptide that includes a first polypeptide comprising the amino acid sequences of SEQ ID NOs: 35, 36, and 37, and a second polypeptide comprising the amino acid sequences of SEQ ID NOs: 38, 39, and 40 is administered to a subject.

In certain embodiments, about 1200 mg of the protein product with a first polypeptide that includes the amino acid sequence of SEQ ID NO: 3, and a second polypeptide that includes the amino acid sequence of SEQ ID NO: 1 is administered to a subject once every two weeks. In certain embodiments, about 1800 mg of the protein product with a first polypeptide that includes the amino acid sequence of SEQ ID NO: 3, and a second polypeptide that includes the amino acid sequence of SEQ ID NO: 1 is administered to a subject once every three weeks. In certain embodiments, about 1200 mg of the protein product that includes a first polypeptide comprising the amino acid sequences of SEQ ID NOs: 35, 36, and 37, and a second polypeptide comprising the amino acid sequences of SEQ ID NOs: 38, 39, and 40 is administered to a subject once every two weeks. In certain embodiments, about 1800 mg of the protein product that includes a first polypeptide comprising the amino acid sequences of SEQ ID NOs: 35, 36, and 37, and a second polypeptide comprising the amino acid sequences of SEQ ID NOs: 38, 39, and 40 is administered to a subject once every three weeks.

### Establishing Body Weight-Independent Dosing Regimen

Informed by the clinical and pre-clinical data, a new, body weight-independent dosing regimen for the administration of anti-PD-L1/TGFβ Trap molecules has been created to achieve less variability in exposure, reduce dosing errors, reduce the time necessary for dose preparation, and reduce drug wastage compared to the mg/kg dosing, thus facilitating favorable treatment outcomes. According to one embodiment, a flat dose of at least 500 mg can be administered, regardless of the patient's body weight. According to another embodiment, a flat dose of at least 1200 mg can be administered, regardless of the patient's body weight. According to another embodiment, a flat dose of 1800 mg can be administered, regardless of the patient's body weight. According to certain embodiments, a flat dose of 2400 mg can be administered, regardless of the patient's body weight. Typically, such doses would be administered repeatedly, such as once every two weeks or once every 3 weeks, for example. For example, a flat dose of 1200 mg can be administered once every two weeks, or a flat dose of 1800 mg can be administered once every three weeks, or a flat dose of 2400 mg can be administered once every three weeks.

### Pharmacokinetic (PK) Analysis Sampling in Humans

An example of pharmacokinetic analysis to determine the optimal flat dose of the anti-PD-L1/TGFβ Trap is provided by the experiments described below.

Serum samples for pharmacokinetic (PK) data analysis were collected before the start of the first dose and at the following time points after the first dose: on Day 1 immediately after the infusion and 4 hours after the start of the infusion; on Day 2 at least 24 hours after the Day 1 end of infusion; and on Days 8 and 15. At selected subsequent dosing occasions pre-dose, end-of-infusion and 2 to 8 hours after the end of infusion samples were collected on days 15, 29, 43. For later time points on days 57, 71 and 85, pre-dose samples were or were to be collected followed by once every 6 weeks PK sampling until 12 weeks, then once every 12 weeks PK sampling. In the expansion phase sparse PK sampling was conducted.

The PK data described above were used to produce a population PK model and to perform simulations of possible dosing regimens. A modeling method, known as the full approach model, described in Gastonguay, M., Full Covariate Models as an Alternative to Methods Relying on Statistical Significance for Inferences about Covariate Effects: A Review of Methodology and 42 Case Studies, (2011) p. 20, Abstract 2229, was applied to the population model data obtained from the simulations to obtain parameters having the following features: 2-compartment PK model with linear elimination, IIV on CL, V1, and V2, combined additive and proportional residual error, full covariate model on CL and Vi. The following baseline covariates were included in the final model: age, weight, sex, race, albumin, CRP, platelet count, eGFR, hepatic impairment, ECOG score, tumor size, tumor type, and previous treatment with biologics. The following estimates of typical parameter estimates of pharmacokinetics of the protein of the present disclosure (e.g., anti-PD-L1/TGFβ Trap) were obtained: clearance (CL) 0.0177 L/h (6.2%), central volume of distribution (V1) 3.64 L (8.81%), peripheral volume of distribution (V2) 0.513 L (25.1%), and inter-compartmental clearance (Q) 0.00219 L/h (17.8%). The inter-patient variability was 22% for CL, 20% for V1, and 135% for V2. Body weight was a relevant covariate on both CL and Vi. To support the flat dosing approach, the impact of the dosing strategy on the exposure variability of the protein of the present disclosure (e.g., anti-PD-L1/TGFβ Trap) was explored. Specifically, simulations were performed to compare the exposure distribution using a flat dosing approach of 1200 mg once every two weeks versus a BW-adjusted dosing approach of either 17.65 mg/kg once every two weeks (corresponding to 1200 mg once every two weeks for a 68 kg subject or 15 mg/kg once every two weeks (corresponding to 1200 mg for a 80 kg subject). Further simulations were performed to compare the exposure distribution using a flat dosing approach of 500 mg once every two weeks versus a BW-adjusted dosing approach of 7.35 mg/kg once every two weeks (corresponding to 500 mg once every two weeks for a 68 kg subject). In addition, simulations were performed to assess the following flat doses at once every three weeks: 1200 mg, 1400, mg, 1600 mg, 1800 mg, 2000 mg, 2200 mg, 2400 mg, 2600 mg, 2800 mg, 3000 mg.

The following methodology for simulations was used: N=200 sets of parameter estimates were drawn from multivariate normal distribution of parameter estimates, using the final PK model variance-covariance matrix. For each parameter estimate, 200 IIV estimates were drawn from $OMEGA multivariate normal distribution, resulting in total 40000 (200×200) subjects. The original dataset (N=380) was resampled with replacement to generate 40000 sets of matched covariates and steady-state exposure metrics (AUC, C_{avg}, C_{trough} and Cₘₐₓ) were generated for each dosing regimen.

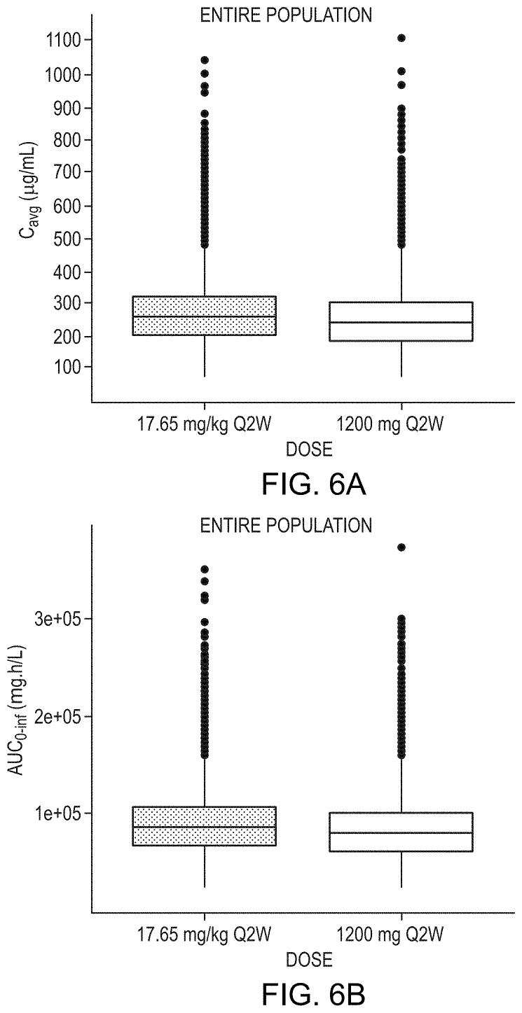

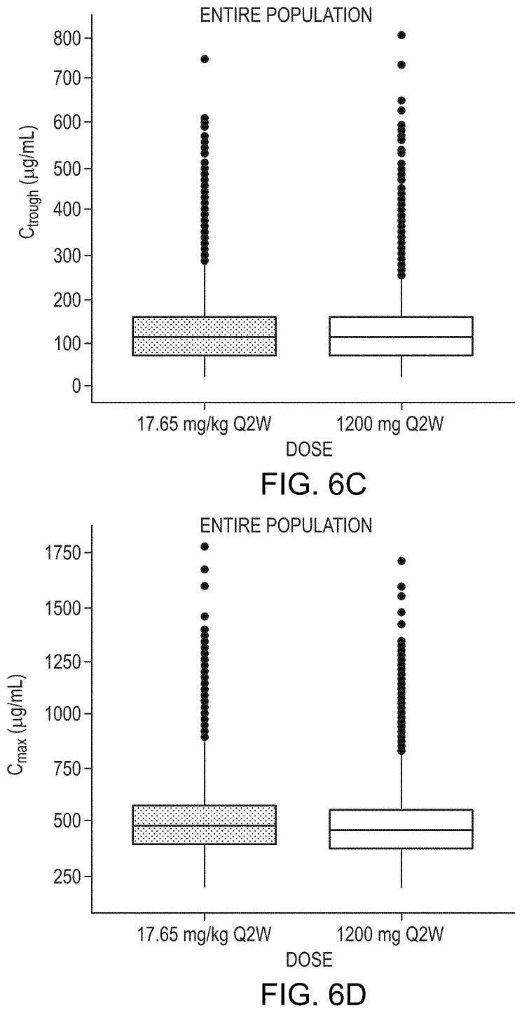

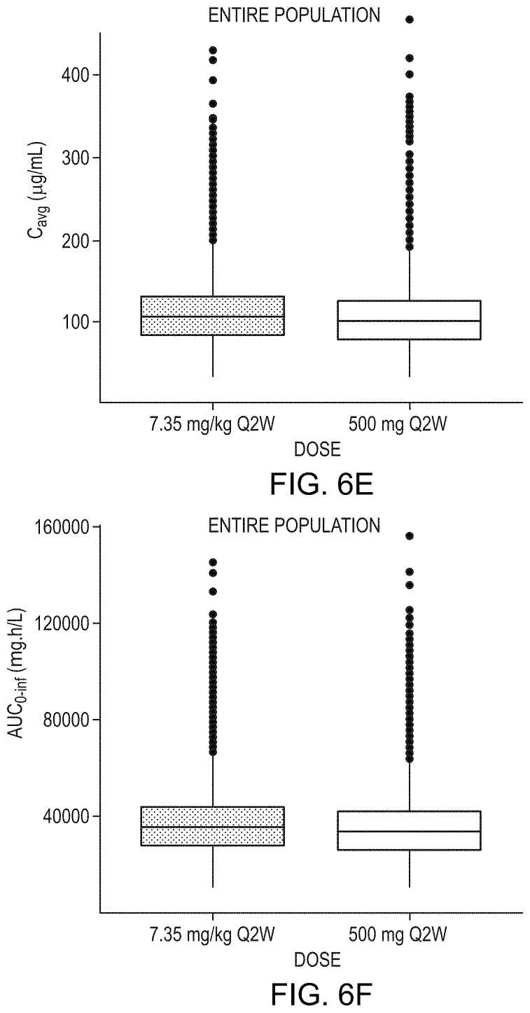

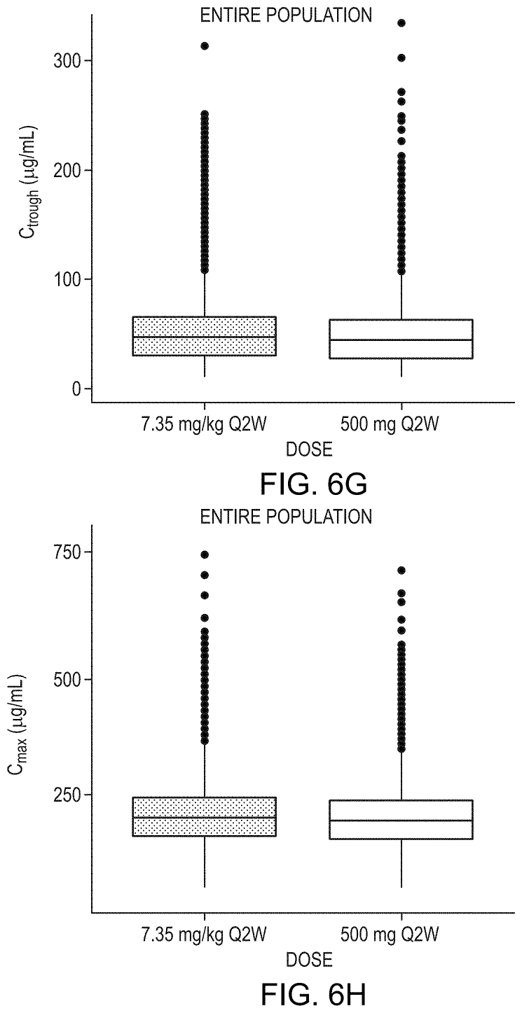

Simulations showed that across a wide BW spectrum, variability in exposure is slightly higher for BW-based dosing in comparison with fixed dosing. An example of exposure distribution at 17.65 mg/kg and 1200 mg flat dose, or 7.35 mg/kg and 500 mg flat dose for a median body weight of 68 kg is shown in FIGS. 6A and 6E, respectively. Simulations also showed the opposite trend in exposure distributions across weight quartiles across the patient population: low-weight patients have higher exposure with fixed dosing, whereas high-weight patients have higher exposure with BW-adjusted dosing.

Establishing Efficacious Dose/Dosing Regimen in Humans: Preliminary Dose-Response in 2^{nd} Line Non-Small-Cell Lung Cancer (2L NSCLC) Following Once Every 2 Weeks (q2w) Dosing of Anti-PD-L1/TGFβ Trap

An example of the therapeutic efficacy of the anti-PD-L1/TGFβ Trap is established by the clinical study described below.

Patients with advanced NSCLC unselected for PD-L who progressed following 1^{st} line standard treatment (no prior immunotherapy) were randomized to receive the anti-PD-L1/TGFβ Trap of the present disclosure at 500 mg or 1200 mg (n=40 per cohort) once every two weeks (q2w), until disease progression, unacceptable toxicity, or trial withdrawal. The primary objective was to assess best overall response (BOR) per Response Evaluation Criteria in Solid Tumors version 1.1 (RECIST v1.1). Other objectives included dose exploration and safety/tolerability assessment. Tumor cell PD-L1 expression levels (Ab clone 73-10 (Dako) [>80%=>50% with Ab clone 22C3 (Dako)]) were characterized as PD-L1<1%, ≥1% (PD-L1+), or ≥80% (PD-L1-high). Tumor cell PD-L1 expression was evaluable in 75 patients.

As of data cut-off at the time of analysis, 80 patients received anti-PD-L1/TGFβ Trap for a median of 11.9 weeks (range, 2-66.1), with a median follow-up of 51.1 weeks. Ten patients remain on treatment. Investigator-assessed confirmed overall response rate (ORR) was 23.8% (500 mg ORR, 20.0%; 1200 mg ORR, 27.5%), with 18 partial responses (PR) seen across both dose levels, and 1 complete response (CR) seen at 1200 mg. As shown in Table 2, clinical activity was observed across PD-L1 expression levels: ORR was 37.0% in PD-L1+ and 85.7% in PD-L1-high patients at 1200 mg. The most common treatment-related adverse events (TRAEs) were pruritus (20.0%), maculopapular rash (18.8%), and decreased appetite (12.5%). Grade 3 TRAEs occurred in 23 patients (28.8%), and Grade 4 TRAEs occurred in 2 patients. Eight patients (500 mg, n=2; 1200 mg, n=6) discontinued treatment due to TRAEs. No treatment-related deaths occurred.

TABLE 2
Observed response rate in 2L NSCLC patients treated with either
500 mg or 1200 mg of anti-PD-L1/TGFβ Trap once every 2 weeks

| ORR | 500 mg | 1200 mg | Total |
|---|---|---|---|
| All, n, % | 8/40, 20.0 | 11/40, 27.5 | 19/80, 23.8 |
| PD-L1+ (≥1%) pts, n, % | 6/31, 19.4 | 11/27, 40.7 | 17/58, 29.3 |
| PD-L1 high (≥80%) pts, n, % | 2/6, 33.3 | 6/7, 85.7 | 8/13, 62.0 |

These results demonstrate that anti-PD-L1/TGFβ Trap monotherapy was well tolerated and showed efficacy across PD-L1 subgroups, with an ORR at 1200 mg of 37.0% and 85.7% in PD-L1+ and PD-L1-high patients, respectively. Given the response rates significantly improved at higher PD-L1 tumor cell expression (e.g., patients treated at 1200 mg), this promising activity of anti-PD-L1/TGFβ Trap observed as a 2L treatment is expected to translate or increase as a first line (1L) therapy in treatment naïve PD-L1-high or PD-L1-independent NSCLC patients.

Establishing Dosing Regimen with Various Dosing Frequencies

Data regimens with various dosing frequencies have been created to allow less frequent administration and/or to allow coordination of dosing schedules with concomitant medications. Specifically, the preliminary population PK modeling and simulation methodology described above has been used to simulate exposures for various dosing regimens and to compare regimens based on exposure.

Based on these simulations, a flat dose of at least 500 mg administered once every two weeks is required to maintain an average concentration of about 100 ag/mL for a typical subject, while a flat dose of about 1200 mg administered once every two weeks is required to maintain a C_{trough} of about 100 μg/mL.

Based on simulations for C_{avg}, 1200 mg once every two weeks is equivalent to 1800 mg once every three weeks, while for C_{trough}, 1200 mg once every two weeks is equivalent to 2800 mg once every three weeks. And for C_{avg}, 500 mg once every two weeks is equivalent to 750 mg once every three weeks; for C_{trough} 500 mg once every two weeks is equivalent to 1,167 mg once every three weeks.

### TGFβ as a Cancer Target

The current disclosure permits localized reduction in TGFβ in a tumor microenvironment by capturing the TGFβ using a soluble cytokine receptor (TGFβRII) tethered to an antibody moiety targeting a cellular immune checkpoint receptor found on the exterior surface of certain tumor cells or immune cells. An example of an antibody moiety of the disclosure to an immune checkpoint protein is anti-PD-L1. The bifunctional molecule of the present disclosure, sometimes referred to herein as an “antibody-cytokine Trap,” is effective precisely because the anti-receptor antibody and cytokine Trap are physically linked. The resulting advantage (over, for example, administration of the antibody and the receptor as separate molecules) is partly because cytokines function predominantly in the local environment through autocrine and paracrine functions. The antibody moiety directs the cytokine Trap to the tumor microenvironment where it can be most effective, by neutralizing the local immunosuppressive autocrine or paracrine effects. Furthermore, in cases where the target of the antibody is internalized upon antibody binding, an effective mechanism for clearance of the cytokine/cytokine receptor complex is provided. Antibody-mediated target internalization was shown for PD-L1, and anti-PD-L1/TGFβ Trap was shown to have a similar internalization rate as anti-PD-L1. This is a distinct advantage over using an anti-TGFβ antibody because first, an anti-TGFβ antibody might not be completely neutralizing; and second, the antibody can act as a carrier extending the half-life of the cytokine.

Indeed, as described below, treatment with the anti-PD-L1/TGFβ Trap elicits a synergistic anti-tumor effect due to the simultaneous blockade of the interaction between PD-L on tumor cells and PD-1 on immune cells, and the neutralization of TGFβ in the tumor microenvironment. Without being bound by theory, this presumably is due to a synergistic effect obtained from simultaneous blocking the two major immune escape mechanisms, and in addition, the depletion of the TGFβ in the tumor microenvironment by a single molecular entity. This depletion is achieved by (1) anti-PD-L1 targeting of tumor cells; (2) binding of the TGFβ autocrine/paracrine in the tumor microenvironment by the TGFβ Trap; and (3) destruction of the bound TGFβ through the PD-L1 receptor-mediated endocytosis. Furthermore, the TGFβRII fused to the C-terminus of Fc (fragment of crystallization of IgG) was several-fold more potent than the TGFβRII-Fc that places the TGFβRII at the N-terminus of Fc.

TGFβ had been a somewhat questionable target in cancer immunotherapy because of its paradoxical roles as the molecular Jekyll and Hyde of cancer (Bierie et al., Nat. Rev. Cancer, 2006; 6:506-20). Like some other cytokines, TGFβ activity is developmental stage and context dependent. Indeed TGFβ can act as either a tumor promoter or a tumor suppressor, affecting tumor initiation, progression and metastasis. The mechanisms underlying this dual role of TGFβ remain unclear (Yang et al., Trends Immunol. 2010; 31:220-227). Although it has been postulated that Smad-dependent signaling mediates the growth inhibition of TGFβ signaling, while the Smad independent pathways contribute to its tumor-promoting effect, there are also data showing that the Smad-dependent pathways are involved in tumor progression (Yang et al., Cancer Res. 2008; 68:9107-11).

Both the TGFβ ligand and the receptor have been studied intensively as therapeutic targets. There are three ligand isoforms, TGFβ1, 2 and 3, all of which exist as homodimers. There are also three TGFβ receptors (TGFβR), which are called TGFβR type I, II and III (López-Casillas et al., J. Cell Biol. 1994; 124:557-68). TGFβRI is the signaling chain and cannot bind ligand. TGFβRII binds the ligand TGFβ1 and 3, but not TGFβ2, with high affinity. The TGFβRII/TGFβ complex recruits TGFβRI to form the signaling complex (Won et al., Cancer Res. 1999; 59:1273-7). TGFβRIII is a positive regulator of TGFβ binding to its signaling receptors and binds all 3 TGFβ isoforms with high affinity. On the cell surface, the TGFβ/TGFβRIII complex binds TGFβ RII and then recruits TGFβRI, which displaces TGFβRIII to form the signaling complex.

Although the three different TGFβ isoforms all signal through the same receptor, they are known to have differential expression patterns and non-overlapping functions in vivo. The three different TGF-β isoform knockout mice have distinct phenotypes, indicating numerous non-compensated functions (Bujak et al., Cardiovasc. Res. 2007; 74:184-95). While TGFβ1 null mice have hematopoiesis and vasculogenesis defects and TGFβ3 null mice display pulmonary development and defective palatogenesis, TGFβ2 null mice show various developmental abnormalities, the most prominent being multiple cardiac deformities (Bartram et al., Circulation 2001; 103:2745-52; Yamagishi et al., Anat. Rec. 2012; 295:257-67). Furthermore, TGFβ is implicated to play a major role in the repair of myocardial damage after ischemia and reperfusion injury. In an adult heart, cardiomyocytes secrete TGFβ, which acts as an autocrine to maintain the spontaneous beating rate. Importantly, 70-85% of the TGFβ secreted by cardiomyocytes is TGFβ2 (Roberts et al., J. Clin. Invest. 1992; 90:2056-62). Despite cardiotoxicity concerns raised by treatment with TGFβRI kinase inhibitors, the present applicant has observed a lack of toxicity, including cardiotoxicity, for anti-PD-L1/TGFβ Trap in monkeys.

Therapeutic approaches to neutralize TGFβ include using the extracellular domains of TGFβ receptors as soluble receptor Traps and neutralizing antibodies. Of the receptor Trap approach, soluble TGFβRIII may seem the obvious choice since it binds all the three TGFβ ligands. However, TGFβRIII, which occurs naturally as a 280-330 kD glucosaminoglycan (GAG)-glycoprotein, with extracellular domain of 762 amino acid residues, is a very complex protein for biotherapeutic development. The soluble TGFβRIII devoid of GAG could be produced in insect cells and has been shown to be a potent TGFβ neutralizing agent (Vilchis-Landeros et al., Biochem. J., (2001), 355:215). The two separate binding domains (the endoglin-related and the uromodulin-related) of TGFβRIII could be independently expressed, but they were shown to have affinities 20 to 100 times lower than that of the soluble TGFβRIII, and much diminished neutralizing activity (Mendoza et al., Biochemistry 2009; 48:11755-65). On the other hand, the extracellular domain of TGFβRII is only 136 amino acid residues in length and can be produced as a glycosylated protein of 25-35 kD. The recombinant soluble TGFβRII was further shown to bind TGFβ1 with a K_{D} of 200 pM, which is fairly similar to the K_{D} of 50 pM for the full length TGFβRII on cells (Lin et al., J. Biol. Chem. 1995; 270:2747-54). Soluble TGFβRII-Fc was tested as an anti-cancer agent and was shown to inhibit established murine malignant mesothelioma growth in a tumor model (Suzuki et al., Clin. Cancer Res., 2004; 10:5907-18). Because TGFβRII does not bind TGFβ2, and TGFβRIII binds TGFβ1 and 3 with lower affinity than TGFβRII, a fusion protein of the endoglin domain of TGFβRIII and extracellular domain of TGFβRII was produced in bacteria and was shown to inhibit the signaling of TGFβ1 and 2 in cell based assays more effectively than either TGFβRII or RIII (Verona et al., Protein Engg. Des. Sel. 2008; 21:463-73).

Still another approach to neutralize all three isoforms of the TGFβ ligands is to screen for a pan-neutralizing anti-TGFβ antibody, or an anti-receptor antibody that blocks the receptor from binding to TGFβ1, 2 and 3. GC1008, a human antibody specific for all isoforms of TGFβ, was in a Phase I/II study in patients with advanced malignant melanoma or renal cell carcinoma (Morris et al., J. Clin. Oncol. 2008; 26:9028 (Meeting abstract)). Although the treatment was found to be safe and well tolerated, only limited clinical efficacy was observed, and hence it was difficult to interpret the importance of anti-TGFβ therapy without further characterization of the immunological effects (Flavell et al., Nat. Rev. Immunol. 2010; 10:554-67). There were also TGFβ-isoform-specific antibodies tested in the clinic. Metelimumab, an antibody specific for TGFβ1, was tested in Phase 2 clinical trial as a treatment to prevent excessive post-operative scarring for glaucoma surgery; and Lerdelimumab, an antibody specific for TGFβ2, was found to be safe but ineffective at improving scarring after eye surgery in a Phase 3 study (Khaw et al., Ophthalmology 2007; 114:1822-1830). Anti-TGFβRII antibodies that block the receptor from binding to all the three TGFβ isoforms, such as the anti-human TGFβRII antibody TR1 and anti-mouse TGFβRII antibody MT1, have also shown some therapeutic efficacy against primary tumor growth and metastasis in mouse models (Zhong et al., Clin. Cancer Res. 2010; 16:1191-205). However, in a recent Phase I study of antibody TR1 (LY3022859), dose escalation beyond 25 mg (flat dose) was considered unsafe due to uncontrolled cytokine release, despite prophylactic treatment (Tolcher et al., Cancer Chemother. Pharmacol. 2017; 79:673-680). To date, the vast majority of the studies on TGFβ targeted anticancer treatment, including small molecule inhibitors of TGFβ signaling that often are quite toxic, are mostly in the preclinical stage and the anti-tumor efficacy obtained has been limited (Calone et al., Exp Oncol. 2012; 34:9-16; Connolly et al., Int. J. Biol. Sci. 2012; 8:964-78).

The antibody-TGFβ Trap of the disclosure is a bifunctional protein containing at least portion of a human TGFβ Receptor II (TGFβRII) that is capable of binding TGFβ. In certain embodiments, the TGFβ Trap polypeptide is a soluble portion of the human TGFβ Receptor Type 2 Isoform A (SEQ ID NO: 8) that is capable of binding TGFβ. In certain embodiments, TGFβ Trap polypeptide contains at least amino acids 73-184 of SEQ ID NO: 8. In certain embodiments, the TGFβ Trap polypeptide contains amino acids 24-184 of SEQ ID NO: 8. In certain embodiments, the TGFβ Trap polypeptide is a soluble portion of the human TGFβ Receptor Type 2 Isoform B (SEQ ID NO: 9) that is capable of binding TGFβ. In certain embodiments, TGFβ Trap polypeptide contains at least amino acids 48-159 of SEQ ID NO: 9. In certain embodiments, the TGFβ Trap polypeptide contains amino acids 24-159 of SEQ ID NO: 9. In certain embodiments, the TGFβ Trap polypeptide contains amino acids 24-105 of SEQ ID NO: 9.

### Mechanisms of Action

The approach of targeting T cell inhibition checkpoints for dis-inhibition with therapeutic antibodies is an area of intense investigation (for a review, see Pardoll, Nat. Rev. Cancer 2012; 12:253-264). In one approach, the antibody moiety or antigen binding fragment thereof targets T cell inhibition checkpoint receptor proteins on the T cell, such as, for example: CTLA-4, PD-1, BTLA, LAG-3, TIM-3, or LAIR1. In another approach, the antibody moiety targets the counter-receptors on antigen presenting cells and tumor cells (which co-opt some of these counter-receptors for their own immune evasion), such as for example: PD-L1 (B7-H1), B7-DC, HVEM, TIM-4, B7-113, or B7-H4.

The disclosure contemplates antibody TGFβ Traps that target, through their antibody moiety or antigen binding fragment thereof, T cell inhibition checkpoints for dis-inhibition. To that end the applicants have tested the anti-tumor efficacy of combining a TGFβ Trap with antibodies targeting various T cell inhibition checkpoint receptor proteins, such as anti-PD-1, anti-PD-L1, anti-TIM-3 and anti-LAG3.

The programmed death 1 (PD-1)/PD-L1 axis is an important mechanism for tumor immune evasion. Effector T cells chronically sensing antigen take on an exhausted phenotype marked by PD-1 expression, a state under which tumor cells engage by upregulating PD-L1. Additionally, in the tumor microenvironment, myeloid cells, macrophages, parenchymal cells and T cells upregulate PD-L1. Blocking the axis restores the effector function in these T cells. Anti-PD-L1/TGFβ Trap also binds TGFβ (1, 2, and 3 isoforms), which is an inhibitory cytokine produced in the tumor microenvironment by cells including apoptotic neutrophils, myeloid-derived suppressor cells, T cells and tumor. Inhibition of TGFβ by soluble TGFβRII reduced malignant mesothelioma in a manner that was associated with increases in CD8+ T cell anti-tumor effects. The absence of TGFβ1 produced by activated CD4+ T cells and Treg cells has been shown to inhibit tumor growth, and protect mice from spontaneous cancer. Thus, TGFβ appears to be important for tumor immune evasion.

TGFβ has growth inhibitory effects on normal epithelial cells, functioning as a regulator of epithelial cell homeostasis, and it acts as a tumor suppressor during early carcinogenesis. As tumors progress toward malignancy, the growth inhibitory effects of TGFβ on the tumor are lost via mutation in one or more TGFβ pathway signaling components or through oncogenic reprogramming. Upon loss of sensitivity to TGFβ inhibition, the tumor continues to produce high levels of TGFβ, which then serve to promote tumor growth. The TGFβ cytokine is overexpressed in various cancer types with correlation to tumor stage. Many types of cells in the tumor microenvironment produce TGFβ including the tumor cells themselves, immature myeloid cells, regulatory T cells, and stromal fibroblasts; these cells collectively generate a large reservoir of TGFβ in the extracellular matrix. TGFβ signaling contributes to tumor progression by promoting metastasis, stimulating angiogenesis, and suppressing innate and adaptive anti-tumor immunity. As a broadly immunosuppressive factor, TGFβ directly down-regulates the effector function of activated cytotoxic T cells and NK cells and potently induces the differentiation of naïve CD4+ T cells to the immunosuppressive regulatory T cells (Treg) phenotype. In addition, TGFβ polarizes macrophages and neutrophils to a wound-healing phenotype that is associated with production of immunosuppressive cytokines. As a therapeutic strategy, neutralization of TGFβ activity has the potential to control tumor growth by restoring effective anti-tumor immunity, blocking metastasis, and inhibiting angiogenesis.

Radiation-induced fibrosis of the lung may occur in lung tissue irradiated at ≥20 Gy within the first 6 months after initiation of treatment. TGFβ is a major profibrotic molecule that contributes to the development of pulmonary fibrosis. Therefore, targeting TGFβ during treatment of locally advanced, unresectable, stage III NSCLC with cCRT might help in countering the detrimental effects of cCRT.

Many pulmonary fibrosis cases are asymptomatic at onset, and early fibrotic alteration in lung tissue with minimal changes can be difficult to distinguish from inflammatory changes in the lung. Symptomatic cases often involve chronic inflammation characterized by high levels of circulating platelet-derived and basic fibroblast growth factor expressed after initial acute inflammation, fibroblast proliferation and migration, release of TGFβ, and collagen deposition in any histologic space of the irradiated lung including the vascular and alveolar compartments. Such chronic inflammation of the lung can lead to ventilation-perfusion mismatch and result in worsening of pulmonary function (or even functional status) as a primary symptom. Other symptoms may be similar to acute-radiation pneumonitis, including nonproductive cough and dyspnea, although these symptoms are generally more chronic in nature. Owing to the pathophysiologic time course, symptoms are not seen until several months after radiation therapy and may continue to progress for years after therapy.

Thus, during treatment of a patient diagnosed with stage III NSCLC with concomitant chemotherapy and radiation therapy, it would be advantageous to spare as much normal lung as possible from radiation-induced damage at the onset of treatment, in order to avoid acute and long term symptomatic lung injury, and for effective cancer therapy.

The present disclosure provides dosage regimens for targeted TGF-β inhibition with an anti-PD-L1/TGFβ Trap molecule for use in a method of treating a treatment naïve subject diagnosed with stage III NSCLC, and/or mitigating pathological conditions, e.g., pulmonary fibrosis, associated with concurrent cCRT. The stage III NSCLC being treated is independent of baseline PD-L1 expression levels. Changes from baseline in lung fibrosis are measured with high resolution CT scan and pulmonary function tests.

Concomitant PD-1 and TGFβ blockade can restore pro-inflammatory cytokines. Anti-PD-L1/TGFβ Trap includes, for example, an extracellular domain of the human TGFβ receptor TGFβRII covalently joined via a glycine/serine linker to the C terminus of each heavy chain of the fully human IgG1 anti-PD-L1 antibody. Given the emerging picture for the anti-PD-1/PD-L1 class, in which responses are apparent but with room for increase in effect size, it is assumed that co-targeting a complementary immune modulation step will improve tumor response. A similar TGF-targeting agent, fresolimumab, which is a monoclonal antibody targeting TGFβ1, 2 and 3, showed initial evidence of tumor response in a Phase I trial in subjects with melanoma.

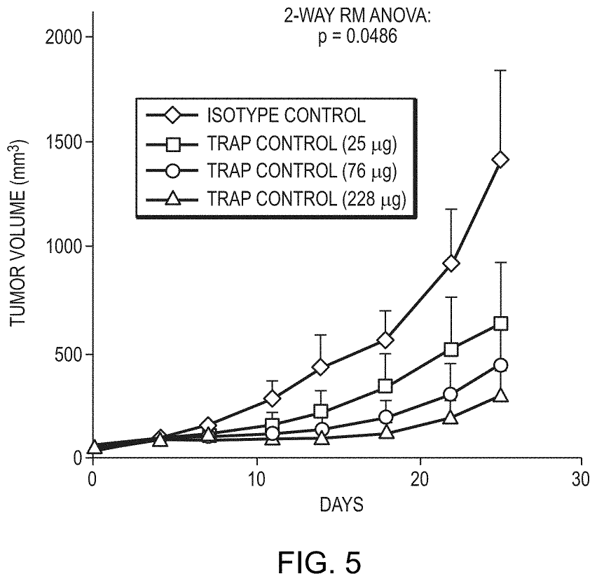

The present disclosure provides experiments that demonstrated that the TGFβRII portion of anti-PD-L1/TGFβ Trap (the Trap control “anti-PDL-1(mut)/TGFβ Trap”) elicited antitumor activity. For example, following subcutaneous implantation in a Detroit 562 human pharyngeal carcinoma model, anti-PDL1(mut)/TGFβ Trap elicited a dose-dependent reduction in tumor volume when administered at 25 μg, 76 μg, or 228 μg (FIG. 5).

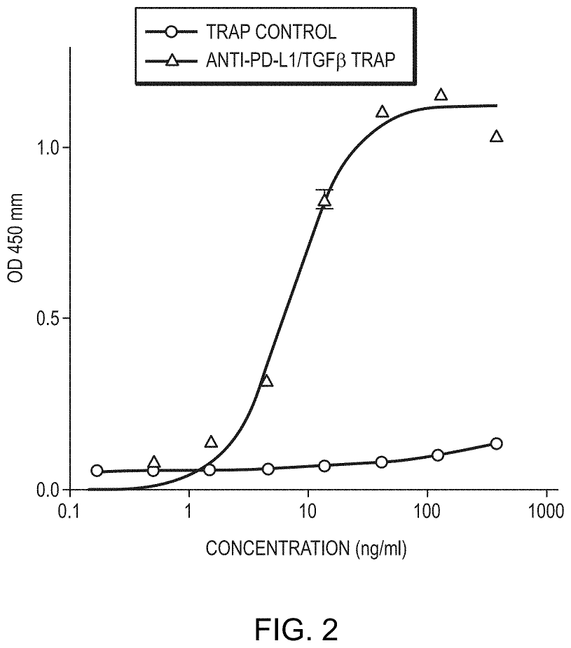

The present disclosure provides experiments that demonstrated that the protein of the present disclosure simultaneously bound to both PD-L1 and TGFβ (FIG. 2).

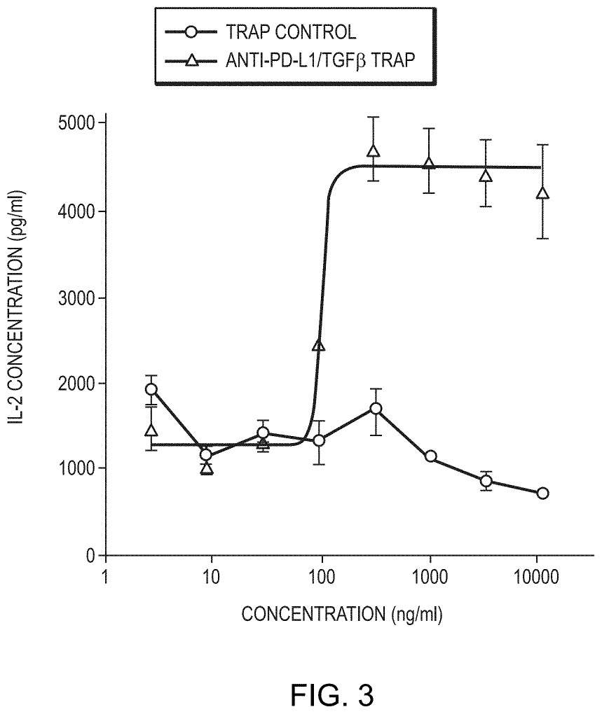

The present disclosure provides experiments that demonstrated that the protein of the present disclosure (e.g. anti-PD-L1/TGFβ Trap) inhibited PD-L1 and TGFβ dependent signaling in vitro. The present disclosure provides experiments that demonstrated that the protein of the present disclosure enhanced T cell effector function in vitro via blockade of PD-L1-mediated immune inhibition as measured by an IL-2 induction assay following superantigen stimulation (FIG. 3). At approximately 100 ng/ml, the protein of the present disclosure induced a dramatic increase in IL-2 levels in vitro (FIG. 3).

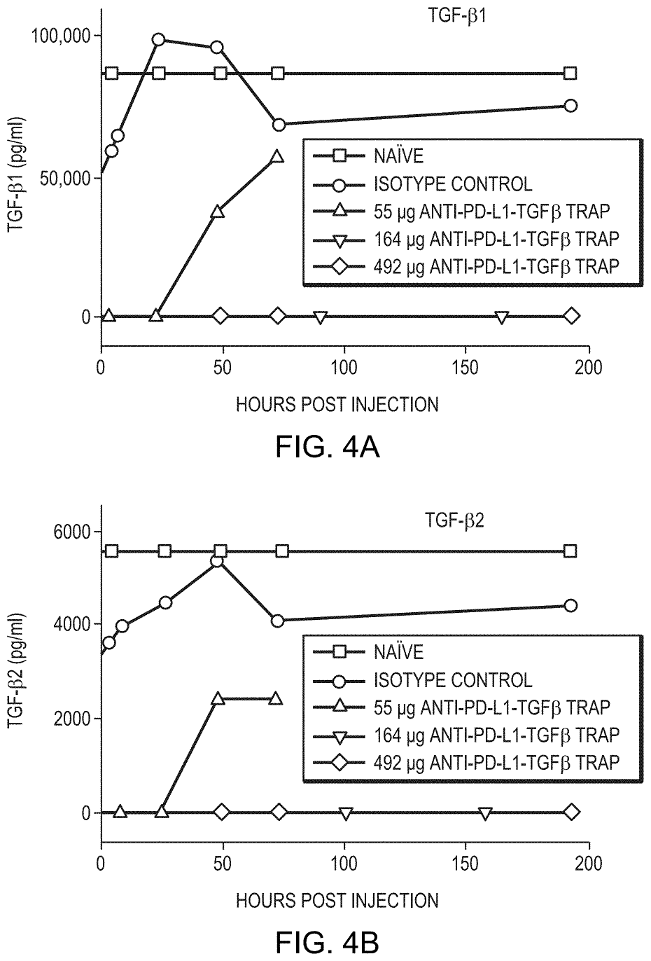

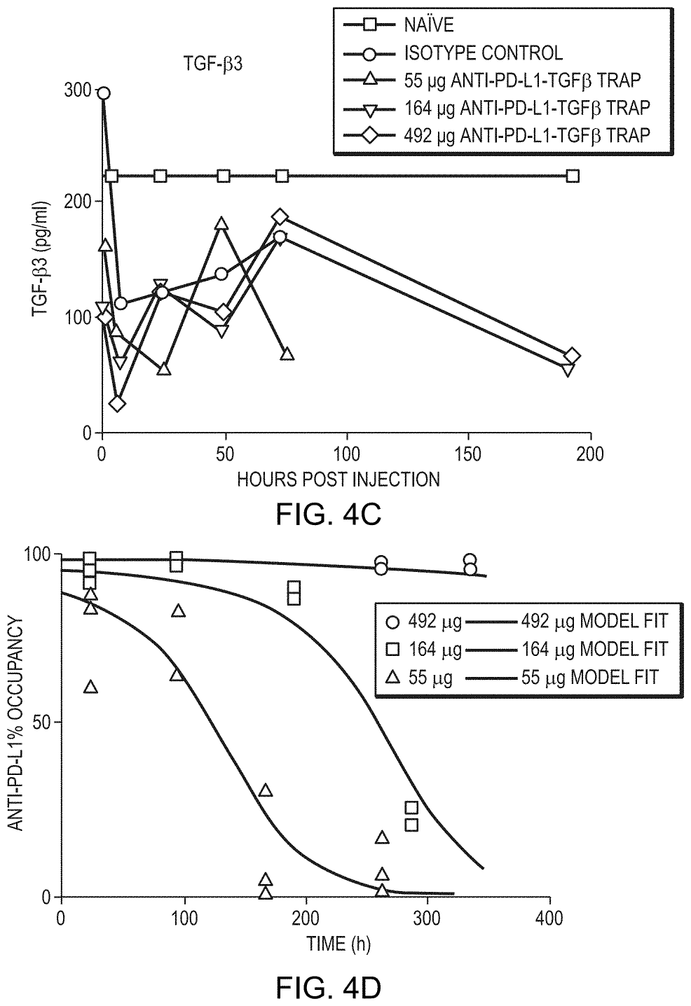

The present disclosure provides experiments that demonstrated that the protein of the present disclosure (e.g. anti-PD-L1/TGFβ Trap) caused depletion of TGFβ from blood in vivo. Treatment of orthotopically implanted EMT-6 breast cancer cells in JH mice with 55 μg, or 164 μg, or 492 μg of the protein of the present disclosure resulted in efficient and specific depletion of TGFβ1 (FIG. 4A), TGFβ2 (FIG. 4B), and TGFβ3 (FIG. 4C). Furthermore, the present disclosure provides experiments that demonstrated that the protein of the present disclosure occupied the PD-L1 target, supporting the notion that that the protein of the present disclosure fit to a receptor binding model in the EMT-6 tumor system (FIG. 4D).

The present disclosure provides experiments that demonstrated that the protein of the present disclosure efficiently, specifically, and simultaneously bound to PD-L1 and TGFβ, possessed potent antitumor activity in a variety of mouse models, suppressed tumor growth and metastasis, as well as extended survival (e.g., survival of up to and including 6 months, 12 months, 18 months, 22 months, 28 months, 32 months, 38 months, 44 months, 50 months, 56 months, 62 months, 68 months, 74 months, 80 months, 86 months, 92 months, 98 months, 104 months, or 110 months) and conferred long-term protective antitumor immunity. In certain embodiments, extended survival is at least 108 months.

### Anti-PD-L1 Antibodies

The anti-PD-L1/TGFβ Trap molecule of the present disclosure can include any anti-PD-L1 antibody, or antigen-binding fragment thereof, described in the art. Anti-PD-L1 antibodies are commercially available, for example, the 29E2A3 antibody (Biolegend, Cat. No. 329701). Antibodies can be monoclonal, chimeric, humanized, or human. Antibody fragments include Fab, F(ab′)2, scFv and Fv fragments, which are described in further detail below.

Exemplary antibodies are described in PCT Publication WO 2013/079174. These antibodies can include a heavy chain variable region polypeptide including an HVR-H1, HVR-H2, and HVR-H3 sequence, where:

(SEQ ID NO: 21)
(a) the HVR-H1 sequence is X₁YX₂MX₃;
(SEQ ID NO: 22)
(b) the HVR-H2 sequence is SIYPSGGX₄TFYADX₅VKG;
(SEQ ID NO: 23)
(c) the HVR-H3 sequence is IKLGTVTTVX₆Y;

further where: X₁ is K, R, T, Q, G, A, W, M, I, or S; X₂ is V, R, K, L, M, or I; X₃ is H, T, N, Q, A, V, Y, W, F, or M; X₄ is F or I; X₅ is S or T; X₆ is E or D.

In a one embodiment, X₁ is M, I, or S; X₂ is R, K, L, M, or I; X₃ is F or M; X₄ is F or I; X₅ is S or T; X₆ is E or D.

In another embodiment X₁ is M, T, or S; X₂ is L, M, or T; X₃ is F or M; X₄ is T; X₅ is S or T; X₆ is D.

In still another embodiment, X₁ is S; X₂ is I; X₃ is M; X₄ is I; X₅ is T; X₆ is D.

In another aspect, the polypeptide further includes variable region heavy chain framework sequences juxtaposed between the HVRs according to the formula: (HC-FR1)-(HVR-H1)-(HC-FR2)-(HVR-H2)-(HC-FR3)-(HVR-H3)-(HC-FR4).

In yet another aspect, the framework sequences are derived from human consensus framework sequences or human germline framework sequences.

In a still further aspect, at least one of the framework sequences is the following:

HC-FR1 is
(SEQ ID NO: 24)
EVQLLESGGGLVQPGGSLRLSCAASGFTFS;
HC-FR2 is
(SEQ ID NO: 25)
WVRQAPGKGLEWVS;
HC-FR3 is
(SEQ ID NO: 26)
RFTISRDNSKNTLYLQMNSLRAEDTAVYYCAR;
HC-FR4 is
(SEQ ID NO: 27)
WGQGTLVTVSS.

In a still further aspect, the heavy chain polypeptide is further combined with a variable region light chain including an HVR-L1, HVR-L2, and HVR-L3, where:

(SEQ ID NO: 28)
(a) the HVR-L1 sequence is TGTX₇X₈DVGX₉YNYVS;
(SEQ ID NO: 29)
(b) the HVR-L2 sequence is X₁₀VX₁₁X₁₂RPS;
(SEQ ID NO: 30)
(c) the HVR-L3 sequence is SSX₁₃TX₁₄X₁₅X₁₆X₁₇RV;

further where: X₇ is N or S; X₈ is T, R, or S; X₉ is A or G; X₁₀ is E or D; X₁₁ is I, N or S; X₁₂ is D, H or N; X₁₃ is F or Y; X₁₄ is N or S; X₁₅ is R, T or S; X₁₆ is G or S; X₁₇ is I or T.

In another embodiment, X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In still another embodiment, X₇ is S; X₈ is S; X₉ is G; X₁₀ is D; X₁₁ is S; X₁₂ is N; X₁₃ is Y; X₁₄ is S; X₁₅ is S; X₁₆ is S; X₁₇ is T.

In a still further aspect, the light chain further includes variable region light chain framework sequences juxtaposed between the HVRs according to the formula: (LC-FR1MHVR-L1)-(LC-FR2)-(HVR-L2)-(LC-FR3)-(HVR-L3)-(LC-FR4).

In a still further aspect, the light chain framework sequences are derived from human consensus framework sequences or human germline framework sequences.

In a still further aspect, the light chain framework sequences are lambda light chain sequences.

In a still further aspect, at least one of the framework sequence is the following:

LC-FR1 is
(SEQ ID NO: 31)
QSALTQPASVSGSPGQSITISC;
LC-FR2 is
(SEQ ID NO: 32)
WYQQHPGKAPKLMIY;
LC-FR3 is
(SEQ ID NO: 33)
GVSNRFSGSKSGNTASLTISGLQAEDEADYYC;
LC-FR4 is
(SEQ ID NO: 34)
FGTGTKVTVL.

In another embodiment, the disclosure provides an anti-PD-L1 antibody or antigen binding fragment including a heavy chain and a light chain variable region sequence, where:

(a) the heavy chain includes an HVR-H1, HVR-H2, and HVR-H3, wherein further: (i) the HVR-H1 sequence is X₁YX₂MX₃ (SEQ ID NO: 21); (ii) the HVR-H2 sequence is SIYPSGGX₄TFYADX₅VKG (SEQ ID NO: 22); (iii) the HVR-H3 sequence is IKLGTVTTVX₆Y (SEQ ID NO: 23), and;

(b) the light chain includes an HVR-L1, HVR-L2, and HVR-L3, wherein further: (iv) the HVR-L1 sequence is TGTX₇X₈DVGX₉YNYVS (SEQ ID NO: 28); (v) the HVR-L2 sequence is X₁₀VX₁₁X₁₂RPS (SEQ ID NO: 29); (vi) the HVR-L3 sequence is SSX₁₃TX₁₄X₁₅X₁₆X₁₇RV (SEQ ID NO: 30); wherein: X₁ is K, R, T, Q, G, A, W, M, I, or S; X₂ is V, R, K, L, M, or I; X₃ is H, T, N, Q, A, V, Y, W, F, or M; X₄ is F or I; X₅ is S or T; X₆ is E or D; X₇ is N or S; X₈ is T, R, or S; X₉ is A or G; X₁₀ is E or D; X₁₁ is I, N, or S; X₁₂ is D, H, or N; X₁₃ is F or Y; X₁₄ is N or S; X₁₅ is R, T, or S; X₁₆ is G or S; X₁₇ is I or T.

In one embodiment, X₁ is M, I, or S; X₂ is R, K, L, M, or I; X₃ is F or M; X₄ is F or I; X₅ is S or T; X₆ is E or D; X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In another embodiment, X₁ is M, I, or S; X₂ is L, M, or T; X₃ is F or M; X₄ is I; X₅ is S or T; X₆ is D; X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In still another embodiment, X₁ is S; X₂ is 1; X₃ is M; X₄ is 1; X₅ is T; X₆ is D; X₇ is S; X₈ is S; X₉ is G; X₁₀ is D; X₁₁ is S; X₁₂ is N; X₁₃ is Y; X₁₄ is S; X₁₅ is S; X₁₆ is S; X₁₇ is T.

In a further aspect, the heavy chain variable region includes one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions include one or more framework sequences juxtaposed between the HVRs as: (LC-FR1 MHVR-L1)-(LC-FR2)-(HVR-L2)-(LC-FR3)-(HVR-L3)-(LC-FR4).

In a still further aspect, the framework sequences are derived from human consensus framework sequences or human germline sequences.

In a still further aspect, one or more of the heavy chain framework sequences is the following:

HC-FR1 is
(SEQ ID NO: 24)
EVQLLESGGGLVQPGGSLRLSCAASGFTFS;
HC-FR2 is
(SEQ ID NO: 25)
WVRQAPGKGLEWVS;
HC-FR3 is
(SEQ ID NO: 26)
RFTISRDNSKNTLYLQMNSLRAEDTAVYYCAR;
HC-FR4 is
(SEQ ID NO: 27)
WGQGTLVTVSS.

In a still further aspect, the light chain framework sequences are lambda light chain sequences.

In a still further aspect, one or more of the light chain framework sequences is the following:

LC-FR1 is
(SEQ ID NO: 31)
QSALTQPASVSGSPGQSITISC;
LC-FR2 is
(SEQ ID NO: 32)
WYQQHPGKAPKLMIY;
LC-FR3 is
(SEQ ID NO: 33)
GVSNRFSGSKSGNTASLTISGLQAEDEADYYC;
LC-FR4 is
(SEQ ID NO: 34)
FGTGTKVTVL.

In a still further aspect, the heavy chain variable region polypeptide, antibody, or antibody fragment further includes at least a C₁₁ domain.

In a more specific aspect, the heavy chain variable region polypeptide, antibody, or antibody fragment further includes a C₁₁1, a C₁₁2, and a C₁₁3 domain.

In a still further aspect, the variable region light chain, antibody, or antibody fragment further includes a C_{L} domain.

In a still further aspect, the antibody further includes a C_{H}1, a C_{H}2, a C_{H}3, and a C_{L} domain.

In a still further specific aspect, the antibody further includes a human or murine constant region.

In a still further aspect, the human constant region is selected from the group consisting of IgG1, IgG2, IgG2, IgG3, IgG4.

In a still further specific aspect, the human or murine constant region is IgG1.

In yet another embodiment, the disclosure features an anti-PD-L1 antibody including a heavy chain and a light chain variable region sequence, where:

(a) the heavy chain includes an HVR-H1, an HVR-H2, and an HVR-H3, having at least 80% overall sequence identity to SYIMM (SEQ ID NO: 35), SIYPSGGITFYADTVKG (SEQ ID NO: 36), and IKLGTVTTVDY (SEQ ID NO: 37), respectively, and

(b) the light chain includes an HVR-L1, an HVR-L2, and an HVR-L3, having at least 80% overall sequence identity to TGTSSDVGGYNYVS (SEQ ID NO: 38), DVSNRPS (SEQ ID NO: 39), and SSYTSSSTRV (SEQ ID NO: 40), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In yet another embodiment, the disclosure features an anti-PD-L1 antibody including a heavy chain and a light chain variable region sequence, where:

(a) the heavy chain includes an HVR-H1, an HVR-H2, and an HVR-H3, having at least 80% overall sequence identity to MYMMM (SEQ ID NO: 41), SIYPSGGITFYADSVKG (SEQ ID NO: 42), and IKLGTVTTVDY (SEQ ID NO: 37), respectively, and

(b) the light chain includes an HVR-L1, an HVR-L2, and an HVR-L3, having at least 80% overall sequence identity to TGTSSDVGAYNYVS (SEQ ID NO: 43), DVSNRPS (SEQ ID NO: 39), and SSYTSSSTRV (SEQ ID NO: 40), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In a still further aspect, in the antibody or antibody fragment according to the disclosure, as compared to the sequences of HVR-H1, HVR-H2, and HVR-H3, at least those amino acids remain unchanged that are highlighted by underlining as follows:

(SEQ ID NO: 35)
(a) in HVR-H1 SYIMM,
(SEQ ID NO: 36)
(b) in HVR-H2 SIYPSGGITFYADTVKG,
(SEQ ID NO: 37)
(c) in HVR-H3 IKLGTVTTVDY;

and further where, as compared to the sequences of HVR-L1, HVR-L2, and HVR-L3 at least those amino acids remain unchanged that are highlighted by underlining as follows:

(SEQ ID NO: 38)
(a) HVR-L1 TGTSSDVGGYNYVS
(SEQ ID NO: 39)
(b) HVR-L2 DVSNRPS
(SEQ ID NO: 40)
(c) HVR-L3 SSYTSSSTRV.

In another aspect, the heavy chain variable region includes one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions include one or more framework sequences juxtaposed between the HVRs as: (LC-FR1)-(HVR-L1)-(LC-FR2)-(HVR-L2)-(LC-FR3)-(HVR-L3)-(LC-FR4).

In yet another aspect, the framework sequences are derived from human germline sequences.

In a still further aspect, one or more of the heavy chain framework sequences is the following:

HC-FR1 is
(SEQ ID NO: 24)
EVQLLESGGGLVQPGGSLRLSCAASGFTFS;
HC-FR2 is
(SEQ ID NO: 25)
WVRQAPGKGLEWVS;
HC-FR3 is
(SEQ ID NO: 26)
RFTISRDNSKNTLYLQMNSLRAEDTAVYYCAR;
HC-FR4 is
(SEQ ID NO: 27)
WGQGTLVTVSS.

In a still further aspect, the light chain framework sequences are derived from a lambda light chain sequence.

In a still further aspect, one or more of the light chain framework sequences is the following:

(SEQ ID NO: 31)
LC-FR1 is QSALTQPASVSGSPGQSITISC;
(SEQ ID NO: 32)
LC-FR2 is WYQQHPGKAPKLMIY;
(SEQ ID NO: 33)
LC-FR3 is GVSNRFSGSKSGNTASLTISGLQAEDEADYYC;
(SEQ ID NO: 34)
LC-FR4 is FGTGTKVTVL.

In a still further specific aspect, the antibody further includes a human or murine constant region.

In a still further aspect, the human constant region is selected from the group consisting of IgG1, IgG2, IgG2, IgG3, IgG4.

In certain embodiments, the disclosure features an anti-PD-L1 antibody including a heavy chain and a light chain variable region sequence, where:

(a) the heavy chain sequence has at least 85% sequence identity to the heavy chain sequence:

(SEQ ID NO: 44)
EVQLLESGGGLVQPGGSLRLSCAASGFTFSSYIMMVWRQAPGKGLEWVSS
IYPSGGITFYADWKGRFTISRDNSKNTLYLQMNSLRAEDTAVYYCARIKL
GTVITVDYWGQGTLVTVSS,

and

(b) the light chain sequence has at least 85% sequence identity to the light chain sequence:

(SEQ ID NO: 45)
QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTRV
FGTGTKVTVL.

In various embodiments, the heavy chain sequence has at least 86% sequence identity to SEQ ID NO: 44 and the light chain sequence has at least 86% sequence identity to SEQ ID NO: 45; the heavy chain sequence has at least 87% sequence identity to SEQ ID NO: 44 and the light chain sequence has at least 87% sequence identity to SEQ ID NO: 45; the heavy chain sequence has at least 88% sequence identity to SEQ ID NO: 44 and the light chain sequence has at least 88% sequence identity to SEQ ID NO: 45; the heavy chain sequence has at least 89% sequence identity to SEQ ID NO: 44 and the light chain sequence has at least 89% sequence identity to SEQ ID NO: 45; the heavy chain sequence has at least, 90% sequence identity to SEQ ID NO: 44 and the light chain sequence has at least 90% sequence identity to SEQ ID NO: 45; the heavy chain sequence has at least 91% sequence identity to SEQ ID NO: 44 and the light chain sequence has at least 91% sequence identity to SEQ ID NO: 45; the heavy chain sequence has at least 92% sequence identity to SEQ ID NO: 44 and the light chain sequence has at least 92% sequence identity to SEQ ID NO: 45; the heavy chain sequence has at least 93% sequence identity to SEQ ID NO: 44 and the light chain sequence has at least 93% sequence identity to SEQ ID NO: 45; the heavy chain sequence has at least 94% sequence identity to SEQ ID NO: 44 and the light chain sequence has at least 94% sequence identity to SEQ ID NO: 45; the heavy chain sequence has at least 95% sequence identity to SEQ ID NO: 44 and the light chain sequence has at least 95% sequence identity to SEQ ID NO: 45; the heavy chain sequence has at least 96% sequence identity to SEQ ID NO: 44 and the light chain sequence has at least 96% sequence identity to SEQ ID NO: 45; the heavy chain sequence has at least 97% sequence identity to SEQ ID NO: 44 and the light chain sequence has at least 97% sequence identity to SEQ ID NO: 45; the heavy chain sequence has at least 98% sequence identity to SEQ ID NO: 44 and the light chain sequence has at least 98% sequence identity to SEQ ID NO: 45; the heavy chain sequence has at least 99% sequence identity to SEQ ID NO: 44 and the light chain sequence has at least 99% sequence identity to SEQ ID NO: 45; or the heavy chain sequence comprises SEQ ID NO: 44 and the light chain sequence comprises SEQ ID NO: 45.

In certain embodiments, the disclosure provides for an anti-PD-L1 antibody including a heavy chain and a light chain variable region sequence, where:

(a) the heavy chain sequence has at least 85% sequence identity to the heavy chain sequence:

(SEQ ID NO: 46)
EVQLLESGGGLVQPGGSLRLSCAASGFTFSMYMMMWVRQAPGKGLEVWSS
IYPSGGITFYADSVKGRFTISRDNSKNTLYLQMNSLRAEDTAIYYCARIK
LGTVTTVDYWGQGTLVTVSS,

and

(b) the light chain sequence has at least 85% sequence identity to the light chain sequence:

(SEQ ID NO: 47)
QSALTQPASVSGSPGQSITISCTGTSSDVGAYNYVSWYQQHPGKAPKLMI
YDVSNRPSGVSNRFSGSKSGNTASLTISGLQAEDEADYYCSSYTSSSTRV
FGTGTKVTVL.

In various embodiments, the heavy chain sequence has at least 86% sequence identity to SEQ ID NO: 46 and the light chain sequence has at least 86% sequence identity to SEQ ID NO: 47; the heavy chain sequence has at least 87% sequence identity to SEQ ID NO: 46 and the light chain sequence has at least 87% sequence identity to SEQ ID NO: 47; the heavy chain sequence has at least 88% sequence identity to SEQ ID NO: 46 and the light chain sequence has at least 88% sequence identity to SEQ ID NO: 47; the heavy chain sequence has at least 89% sequence identity to SEQ ID NO: 46 and the light chain sequence has at least 89% sequence identity to SEQ ID NO: 47; the heavy chain sequence has at least, 90% sequence identity to SEQ ID NO: 46 and the light chain sequence has at least 90% sequence identity to SEQ ID NO: 47; the heavy chain sequence has at least 91% sequence identity to SEQ ID NO: 46 and the light chain sequence has at least 91% sequence identity to SEQ ID NO: 47; the heavy chain sequence has at least 92% sequence identity to SEQ ID NO: 46 and the light chain sequence has at least 92% sequence identity to SEQ ID NO: 47; the heavy chain sequence has at least 93% sequence identity to SEQ ID NO: 46 and the light chain sequence has at least 93% sequence identity to SEQ ID NO: 47; the heavy chain sequence has at least 94% sequence identity to SEQ ID NO: 46 and the light chain sequence has at least 94% sequence identity to SEQ ID NO: 47; the heavy chain sequence has at least 95% sequence identity to SEQ ID NO: 46 and the light chain sequence has at least 95% sequence identity to SEQ ID NO: 47; the heavy chain sequence has at least 96% sequence identity to SEQ ID NO: 46 and the light chain sequence has at least 96% sequence identity to SEQ ID NO: 47; the heavy chain sequence has at least 97% sequence identity to SEQ ID NO: 46 and the light chain sequence has at least 97% sequence identity to SEQ ID NO: 47; the heavy chain sequence has at least 98% sequence identity to SEQ ID NO: 46 and the light chain sequence has at least 98% sequence identity to SEQ ID NO: 47; the heavy chain sequence has at least 99% sequence identity to SEQ ID NO: 46 and the light chain sequence has at least 99% sequence identity to SEQ ID NO: 47; or the heavy chain sequence comprises SEQ ID NO: 46 and the light chain sequence comprises SEQ ID NO: 47.

In another embodiment the antibody binds to human, mouse, or cynomolgus monkey PD-L1. In a specific aspect the antibody is capable of blocking the interaction between human, mice, or cynomolgus monkey PD-1 and the respective human, mouse, or cynomolgus monkey PD-1 receptors.

In another embodiment, the antibody binds to human PD-L1 with a KD of 5×10-9 M or less, preferably with a KD of 2×10-9 M or less, and even more preferred with a KD of 1×10-9 M or less.

In yet another embodiment, the disclosure relates to an anti-PD-L1 antibody or antigen binding fragment thereof which binds to a functional epitope including residues Y56 and D61 of human PD-L1.

In a specific aspect, the functional epitope further includes E58, E60, Q66, R113, and M115 of human PD-L1.

In a more specific aspect, the antibody binds to a conformational epitope, including residues 54-66 and 112-122 of human PD-L1.

In certain embodiments, the disclosure is related to an anti-PD-L1 antibody, or antigen binding fragment thereof, which cross-competes for binding to PD-L1 with an antibody according to the disclosure as described herein.

In certain embodiments, the disclosure features proteins and polypeptides including any of the above described anti-PD-L1 antibodies in combination with at least one pharmaceutically acceptable carrier.

In certain embodiments, the disclosure features an isolated nucleic acid encoding a polypeptide, or light chain or a heavy chain variable region sequence of an anti-PD-L1 antibody, or antigen binding fragment thereof, as described herein. In certain embodiments, the disclosure provides for an isolated nucleic acid encoding a light chain or a heavy chain variable region sequence of an anti-PD-L1 antibody, wherein:

(a) the heavy chain includes an HVR-H1, an HVR-112, and an HVR-H3 sequence having at least 80% sequence identity to SYIMM (SEQ ID NO: 35), SIYPSGGITFYADTVKG (SEQ ID NO: 36), and IKLGTVTTVDY (SEQ ID NO: 37), respectively, or

(b) the light chain includes an HVR-L1, an HVR-L2, and an HVR-L3 sequence having at least 80% sequence identity to TGTSSDVGGYNYVS (SEQ ID NO: 38), DVSNRPS (SEQ ID NO: 39), and SSYTSSSTRV (SEQ ID NO: 40), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In a further aspect, the nucleic acid sequence for the heavy chain is:

| (SEQ ID NO: 48) | |
|---|---|
| atggagttgc ctgttaggct gttggtgctg atgttctgga ttcctgctag ctccagcgag | 60 |
| gtgcagctgc tggaatccgg cggaggactg gtgcagcctg gcggctccct gagactgtct | 120 |
| tgcgccgcct ccggcttcac cttctccagc tacatcatga tgtgggtgcg acaggcccct | 180 |
| ggcaagggcc tggaatgggt gtcctccatc tacccctccg gcggcatcac cttctacgcc | 240 |
| gacaccgtga agggccggtt caccatctcc cgggacaact ccaagaacac cctgtacctg | 300 |
| cagatgaact ccctgcgggc cgaggacacc gccgtgtact actgcgcccg gatcaagctg | 360 |
| ggcaccgtga ccaccgtgga ctactggggc cagggcaccc tggtgacagt gtcctccgcc | 420 |
| tccaccaagg gcccatcggt cttccccctg gcaccctcct ccaagagcac ctctgggggc | 480 |
| acagcggccc tgggctgcct ggtcaaggac tacttccccg aaccggtgac ggtgtcgtgg | 540 |
| aactcaggcg ccctgaccag cggcgtgcac accttcccgg ctgtcctaca gtcctcagga | 600 |
| ctctactccc tcagcagcgt ggtgaccgtg ccctccagca gcttgggcac ccagacctac | 660 |
| atctgcaacg tgaatcacaa gcccagcaac accaaggtgg acaagaaagt tgagcccaaa | 720 |
| tcttgtgaca aaactcacac atgcccaccg tgcccagcac ctgaactcct ggggggaccg | 780 |
| tcagtcttcc tcttcccccc aaaacccaag gacaccctca tgatctcccg gacccctgag | 840 |
| gtcacatgcg tggtggtgga cgtgagccac gaagaccctg aggtcaagtt caactggtac | 900 |
| gtggacggcg tggaggtgca taatgccaag acaaagccgc gggaggagca gtacaacagc | 960 |
| acgtaccgtg tggtcagcgt cctcaccgtc ctgcaccagg actggctgaa tggcaaggag | 1020 |
| tacaagtgca aggtctccaa caaagccctc ccagccccca tcgagaaaac catctccaaa | 1080 |
| gccaaagggc agccccgaga accacaggtg tacaccctgc ccccatcacg ggatgagctg | 1140 |
| accaagaacc aggtcagcct gacctgcctg gtcaaaggct tctatcccag cgacatcgcc | 1200 |
| gtggagtggg agagcaatgg gcagccggag aacaactaca agaccacgcc tcccgtgctg | 1260 |
| gactccgacg gctccttctt cctctatagc aagctcaccg tggacaagag caggtggcag | 1320 |
| caggggaacg tcttctcatg ctccgtgatg catgaggctc tgcacaacca ctacacgcag | 1380 |
| aagagcctct ccctgtcccc gggtaaa | 1407 |

and the nucleic acid sequence for the light chain is:

| (SEQ ID NO: 49) | |
|---|---|
| atggagttgc ctgttaggct gttggtgctg atgttctgga ttcctgcttc cttaagccag | 60 |
| tccgccctga cccagcctgc ctccgtgtct ggctcccctg gccagtccat caccatcagu | 120 |
| tgcaccggca cctccagcga cgtgggcggc tacaactacg tgtcctggta tcagcagcac | 180 |
| cccggcaagg cccccaagct gatgatctac gacgtgtcca accggccctc cggcgtgtcc | 240 |
| aacagattct ccggctccaa gtccggcaac accgcctccc tgaccatcag cggactgcag | 300 |
| gcagaggacg aggccgacta ctactgctcc tcctacacct cctccagcac cagagtgttc | 360 |
| ggcaccggca caaaagtgac cgtgctgggc cagcccaagg ccaacccaac cgtgacactg | 420 |
| ttccccccat cctccgagga actgcaggcc aacaaggcca ccctggtctg cctgatctca | 480 |
| gatttctatc caggcgccgt gaccgtggcc tggaaggctg atggctcccc agtgaaggcc | 540 |
| ggcgtggaaa ccaccaagcc ctccaagcag tccaacaaca aatacgccgc ctcctcctac | 600 |
| ctgtccctga cccccgagca gtggaagtcc caccggtcct acagctgcca ggtcacacac | 660 |
| gagggctcca ccgtggaaaa gaccgtcgcc cccaccgagt gctca. | 705 |

Further exemplary anti-PD-1 antibodies that can be used in an anti-PD-L1/TGFβ Trap are described in US patent application publication US 2010/0203056. In one embodiment of the disclosure, the antibody moiety is YW243.55S70. In another embodiment of the disclosure, the antibody moiety is MPDL3289A.

In certain embodiments, the disclosure features an anti-PD-L1 antibody moiety including a heavy chain and a light chain variable region sequence, where:

(a) the heavy chain sequence has at least 85% sequence identity to the heavy chain sequence:

(SEQ ID NO: 12)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSS,

and

(b) the light chain sequence has at least 85% sequence identity to the light chain sequence:

(SEQ ID NO: 13)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR

In various embodiments, the heavy chain sequence has at least 86% sequence identity to SEQ ID NO: 12 and the light chain sequence has at least 86% sequence identity to SEQ ID NO: 13; the heavy chain sequence has at least 87% sequence identity to SEQ ID NO: 12 and the light chain sequence has at least 87% sequence identity to SEQ ID NO: 13; the heavy chain sequence has at least 88% sequence identity to SEQ ID NO: 12 and the light chain sequence has at least 88% sequence identity to SEQ ID NO: 13; the heavy chain sequence has at least 89% sequence identity to SEQ ID NO: 12 and the light chain sequence has at least 89% sequence identity to SEQ ID NO: 13; the heavy chain sequence has at least, 90% sequence identity to SEQ ID NO: 12 and the light chain sequence has at least 90% sequence identity to SEQ ID NO: 13; the heavy chain sequence has at least 91% sequence identity to SEQ ID NO: 12 and the light chain sequence has at least 91% sequence identity to SEQ ID NO: 13; the heavy chain sequence has at least 92% sequence identity to SEQ ID NO: 12 and the light chain sequence has at least 92% sequence identity to SEQ ID NO: 13; the heavy chain sequence has at least 93% sequence identity to SEQ ID NO: 12 and the light chain sequence has at least 93% sequence identity to SEQ ID NO: 13; the heavy chain sequence has at least 94% sequence identity to SEQ ID NO: 12 and the light chain sequence has at least 94% sequence identity to SEQ ID NO: 13; the heavy chain sequence has at least 95% sequence identity to SEQ ID NO: 12 and the light chain sequence has at least 95% sequence identity to SEQ ID NO: 13; the heavy chain sequence has at least 96% sequence identity to SEQ ID NO: 12 and the light chain sequence has at least 96% sequence identity to SEQ ID NO: 13; the heavy chain sequence has at least 97% sequence identity to SEQ ID NO: 12 and the light chain sequence has at least 97% sequence identity to SEQ ID NO: 13; the heavy chain sequence has at least 98% sequence identity to SEQ ID NO: 12 and the light chain sequence has at least 98% sequence identity to SEQ ID NO: 13; the heavy chain sequence has at least 99% sequence identity to SEQ ID NO: 12 and the light chain sequence has at least 99% sequence identity to SEQ ID NO: 13; or the heavy chain sequence comprises SEQ ID NO: 12 and the light chain sequence comprises SEQ ID NO: 13.

In certain embodiments, the disclosure features an anti-PD-L1 antibody moiety including a heavy chain and a light chain variable region sequence, where:

(a) the heavy chain sequence has at least 85% sequence identity to the heavy chain sequence:

(SEQ ID NO: 14)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSA,

and

(b) the light chain sequence has at least 85% sequence identity to the light chain sequence:

(SEQ ID NO: 13)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR

In various embodiments, the heavy chain sequence has at least 86% sequence identity to SEQ ID NO: 14 and the light chain sequence has at least 86% sequence identity to SEQ ID NO: 13; the heavy chain sequence has at least 87% sequence identity to SEQ ID NO: 14 and the light chain sequence has at least 87% sequence identity to SEQ ID NO: 13; the heavy chain sequence has at least 88% sequence identity to SEQ ID NO: 14 and the light chain sequence has at least 88% sequence identity to SEQ ID NO: 13; the heavy chain sequence has at least 89% sequence identity to SEQ ID NO: 14 and the light chain sequence has at least 89% sequence identity to SEQ ID NO: 13; the heavy chain sequence has at least, 90% sequence identity to SEQ ID NO: 14 and the light chain sequence has at least 90% sequence identity to SEQ ID NO: 13; the heavy chain sequence has at least 91% sequence identity to SEQ ID NO: 14 and the light chain sequence has at least 91% sequence identity to SEQ ID NO: 13; the heavy chain sequence has at least 92% sequence identity to SEQ ID NO: 14 and the light chain sequence has at least 92% sequence identity to SEQ ID NO: 13; the heavy chain sequence has at least 93% sequence identity to SEQ ID NO: 14 and the light chain sequence has at least 93% sequence identity to SEQ ID NO: 13; the heavy chain sequence has at least 94% sequence identity to SEQ ID NO: 14 and the light chain sequence has at least 94% sequence identity to SEQ ID NO: 13; the heavy chain sequence has at least 95% sequence identity to SEQ ID NO: 14 and the light chain sequence has at least 95% sequence identity to SEQ ID NO: 13; the heavy chain sequence has at least 96% sequence identity to SEQ ID NO: 14 and the light chain sequence has at least 96% sequence identity to SEQ ID NO: 13; the heavy chain sequence has at least 97% sequence identity to SEQ ID NO: 14 and the light chain sequence has at least 97% sequence identity to SEQ ID NO: 13; the heavy chain sequence has at least 98% sequence identity to SEQ ID NO: 14 and the light chain sequence has at least 98% sequence identity to SEQ ID NO: 13; the heavy chain sequence has at least 99% sequence identity to SEQ ID NO: 14 and the light chain sequence has at least 99% sequence identity to SEQ ID NO: 13; or the heavy chain sequence comprises SEQ ID NO: 14 and the light chain sequence comprises SEQ ID NO: 13.

Further exemplary anti-PD-L1 antibodies that can be used in an anti-PD-L1/TGFβ Trap are described in US patent application publication US 2018/0334504.

In certain embodiments, the disclosure features an anti-PD-L1 antibody moiety including a heavy chain and a light chain variable region sequence, where

(a) the heavy chain sequence has at least 85% sequence identity to the heavy chain sequence:

(SEQ ID NO: 55)
QVQLQESGPGLVKPSQTLSLTCTVSGGSISNDYWTWIRQHPGKGLEYIG
YISYTGSTYYNPSLKSRVTISRDTSKNQFSLKLSSVTAADTAVYYCARS
GGWLAPFDYVVGRGTLVTVSS,

and

(b) the light chain sequence has at least 85% sequence identity to the light chain sequence:

(SEQ ID NO: 56)
DIVMTQSPDSLAVSLGERATINCKSSQSLFYHSNQKHSLAWYQQKPGQP
PKLLIYGASTRESGVPDRFSGSGSGTDFTLTISSLQAEDVAVYYCQQYY
GYPYTFGGGTKVEIK.

In various embodiments, the heavy chain sequence has at least 86% sequence identity to SEQ ID NO: 55 and the light chain sequence has at least 86% sequence identity to SEQ ID NO: 56; the heavy chain sequence has at least 87% sequence identity to SEQ ID NO: 55 and the light chain sequence has at least 87% sequence identity to SEQ ID NO: 56; the heavy chain sequence has at least 88% sequence identity to SEQ ID NO: 55 and the light chain sequence has at least 88% sequence identity to SEQ ID NO: 56; the heavy chain sequence has at least 89% sequence identity to SEQ ID NO: 55 and the light chain sequence has at least 89% sequence identity to SEQ ID NO: 56; the heavy chain sequence has at least, 90% sequence identity to SEQ ID NO: 55 and the light chain sequence has at least 90% sequence identity to SEQ ID NO: 56; the heavy chain sequence has at least 91% sequence identity to SEQ ID NO: 55 and the light chain sequence has at least 91% sequence identity to SEQ ID NO: 56; the heavy chain sequence has at least 92% sequence identity to SEQ ID NO: 55 and the light chain sequence has at least 92% sequence identity to SEQ ID NO: 56; the heavy chain sequence has at least 93% sequence identity to SEQ ID NO: 55 and the light chain sequence has at least 93% sequence identity to SEQ ID NO: 56; the heavy chain sequence has at least 94% sequence identity to SEQ ID NO: 55 and the light chain sequence has at least 94% sequence identity to SEQ ID NO: 56; the heavy chain sequence has at least 95% sequence identity to SEQ ID NO: 55 and the light chain sequence has at least 95% sequence identity to SEQ ID NO: 56; the heavy chain sequence has at least 96% sequence identity to SEQ ID NO: 55 and the light chain sequence has at least 96% sequence identity to SEQ ID NO: 56; the heavy chain sequence has at least 97% sequence identity to SEQ ID NO: 55 and the light chain sequence has at least 97% sequence identity to SEQ ID NO: 56; the heavy chain sequence has at least 98% sequence identity to SEQ ID NO: 55 and the light chain sequence has at least 98% sequence identity to SEQ ID NO: 56; the heavy chain sequence has at least 99% sequence identity to SEQ ID NO: 55 and the light chain sequence has at least 99% sequence identity to SEQ ID NO: 56; or the heavy chain sequence comprises SEQ ID NO: 55 and the light chain sequence comprises SEQ ID NO: 56.

In certain embodiments, the disclosure features an anti-PD-L1 antibody moiety including a heavy chain and a light chain variable region sequence, where

(a) the heavy chain sequence has at least 85% sequence identity to the heavy chain sequence:

(SEQ ID NO: 57)
QVQLVQSGAEVKKPGASVKVSCKASGYTFTSYWMHWVRQAPGQGLEWMG
RIGPNSGFTSYNEKFIKNRVTMTRDTSTSTVYMELSSLRSEDTAVYYCA
RGGSSYDYFDYWGQGTTVTVSS,

and

(b) the light chain sequence has at least 85% sequence identity to the light chain sequence:

(SEQ ID NO: 58)
DIVLTQSPASLAVSPGQRATITCRASESVSIHGTHLMHWYQQKPGQPPK
LLIYAASNLESGVPARFSGSGSGTDFTLTINPVEAEDTANYYCQQSFED
PLTFGQGTKLEIK.

In various embodiments, the heavy chain sequence has at least 86% sequence identity to SEQ ID NO: 57 and the light chain sequence has at least 86% sequence identity to SEQ ID NO: 58; the heavy chain sequence has at least 87% sequence identity to SEQ ID NO: 57 and the light chain sequence has at least 87% sequence identity to SEQ ID NO: 58; the heavy chain sequence has at least 88% sequence identity to SEQ ID NO: 57 and the light chain sequence has at least 88% sequence identity to SEQ ID NO: 58; the heavy chain sequence has at least 89% sequence identity to SEQ ID NO: 57 and the light chain sequence has at least 89% sequence identity to SEQ ID NO: 58; the heavy chain sequence has at least, 90% sequence identity to SEQ ID NO: 57 and the light chain sequence has at least 90% sequence identity to SEQ ID NO: 58; the heavy chain sequence has at least 91% sequence identity to SEQ ID NO: 57 and the light chain sequence has at least 91% sequence identity to SEQ ID NO: 58; the heavy chain sequence has at least 92% sequence identity to SEQ ID NO: 57 and the light chain sequence has at least 92% sequence identity to SEQ ID NO: 58; the heavy chain sequence has at least 93% sequence identity to SEQ ID NO: 57 and the light chain sequence has at least 93% sequence identity to SEQ ID NO: 58; the heavy chain sequence has at least 94% sequence identity to SEQ ID NO: 57 and the light chain sequence has at least 94% sequence identity to SEQ ID NO: 58; the heavy chain sequence has at least 95% sequence identity to SEQ ID NO: 57 and the light chain sequence has at least 95% sequence identity to SEQ ID NO: 58; the heavy chain sequence has at least 96% sequence identity to SEQ ID NO: 57 and the light chain sequence has at least 96% sequence identity to SEQ ID NO: 58; the heavy chain sequence has at least 97% sequence identity to SEQ ID NO: 57 and the light chain sequence has at least 97% sequence identity to SEQ ID NO: 58; the heavy chain sequence has at least 98% sequence identity to SEQ ID NO: 57 and the light chain sequence has at least 98% sequence identity to SEQ ID NO: 58; the heavy chain sequence has at least 99% sequence identity to SEQ ID NO: 57 and the light chain sequence has at least 99% sequence identity to SEQ ID NO: 58; or the heavy chain sequence comprises SEQ ID NO: 57 and the light chain sequence comprises SEQ ID NO: 58.

In certain embodiments, the disclosure features an anti-PD-L1 antibody moiety including a heavy chain and a light chain sequence, where

(a) the heavy chain sequence has at least 85% sequence identity to the heavy chain sequence:

(SEQ ID NO: 59)
QVQLQESGPGLVKPSQTLSLTCTVSGGSISNDYWTWIRQHPGKGLEYIG
YISYTGSTYYNPSLKSRVTISRDTSKNQFSLKLSSVTAADTAVYYCARS
GGWLAPFDYWGRGTLVTVSSASTKGPSVFPLAPCSRSTSESTAALGCLV
KDYFPEPVTVSWNSGALTSGVHTFPAVLQSSGLYSLSSVVTVPSSSLGT
KTYTCNVDHKPSNTKVDKRVESKYGPPCPPCPAPEAAGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGK,

and

(b) the light chain sequence has at least 85% sequence identity to the light chain sequence:

(SEQ ID NO: 60)
DIVMTQSPDSLAVSLGERATINCKSSQSLFYHSNQKHSLAWYQQKPGQPP
KLLIYGASTRESGVPDRFSGSGSGTDFTLTISSLQAEDVAVYYCQQYYGY
PYTFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC.

In various embodiments, the heavy chain sequence has at least 86% sequence identity to SEQ ID NO: 59 and the light chain sequence has at least 86% sequence identity to SEQ ID NO: 60; the heavy chain sequence has at least 87% sequence identity to SEQ ID NO: 59 and the light chain sequence has at least 87% sequence identity to SEQ ID NO: 60; the heavy chain sequence has at least 88% sequence identity to SEQ ID NO: 59 and the light chain sequence has at least 88% sequence identity to SEQ ID NO: 60; the heavy chain sequence has at least 89% sequence identity to SEQ ID NO: 59 and the light chain sequence has at least 89% sequence identity to SEQ ID NO: 60; the heavy chain sequence has at least, 90% sequence identity to SEQ ID NO: 59 and the light chain sequence has at least 90% sequence identity to SEQ ID NO: 60; the heavy chain sequence has at least 91% sequence identity to SEQ ID NO: 59 and the light chain sequence has at least 91% sequence identity to SEQ ID NO: 60; the heavy chain sequence has at least 92% sequence identity to SEQ ID NO: 59 and the light chain sequence has at least 92% sequence identity to SEQ ID NO: 60; the heavy chain sequence has at least 93% sequence identity to SEQ ID NO: 59 and the light chain sequence has at least 93% sequence identity to SEQ ID NO: 60; the heavy chain sequence has at least 94% sequence identity to SEQ ID NO: 59 and the light chain sequence has at least 94% sequence identity to SEQ ID NO: 60; the heavy chain sequence has at least 95% sequence identity to SEQ ID NO: 59 and the light chain sequence has at least 95% sequence identity to SEQ ID NO: 60; the heavy chain sequence has at least 96% sequence identity to SEQ ID NO: 59 and the light chain sequence has at least 96% sequence identity to SEQ ID NO: 60; the heavy chain sequence has at least 97% sequence identity to SEQ ID NO: 59 and the light chain sequence has at least 97% sequence identity to SEQ ID NO: 60; the heavy chain sequence has at least 98% sequence identity to SEQ ID NO: 59 and the light chain sequence has at least 98% sequence identity to SEQ ID NO: 60; the heavy chain sequence has at least 99% sequence identity to SEQ ID NO: 59 and the light chain sequence has at least 99% sequence identity to SEQ ID NO: 60; or the heavy chain sequence comprises SEQ ID NO: 59 and the light chain sequence comprises SEQ ID NO: 60.

In certain embodiments, the disclosure features an anti-PD-L1 antibody moiety including a heavy chain and a light chain sequence, where

(a) the heavy chain sequence has at least 85% sequence identity to the heavy chain sequence:

(SEQ ID NO: 61)
QVQLVQSGAEVKKPGASVKVSCKASGYTFTSYWMHWVRQAPGQGLEWMGR
IGPNSGFTSYNEKFIKNRVTMTRDTSTSTVYMELSSLRSEDTAVYYCARG
GSSYDYFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEAAGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGA,

and

(b) the light chain sequence has at least 85% sequence identity to the light chain sequence:

(SEQ ID NO: 62)
DIVLTQSPASLAVSPGQRATITCRASESVSIHGTHLMHWYQQKPGQPPKL
LIYAASNLESGVPARFSGSGSGTDFTLTINPVEAEDTANYYCQQSFEDPL
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

In various embodiments, the heavy chain sequence has at least 86% sequence identity to SEQ ID NO: 61 and the light chain sequence has at least 86% sequence identity to SEQ ID NO: 62; the heavy chain sequence has at least 87% sequence identity to SEQ ID NO: 61 and the light chain sequence has at least 87% sequence identity to SEQ ID NO: 62; the heavy chain sequence has at least 88% sequence identity to SEQ ID NO: 61 and the light chain sequence has at least 88% sequence identity to SEQ ID NO: 62; the heavy chain sequence has at least 89% sequence identity to SEQ ID NO: 61 and the light chain sequence has at least 89% sequence identity to SEQ ID NO: 62; the heavy chain sequence has at least, 90% sequence identity to SEQ ID NO: 61 and the light chain sequence has at least 90% sequence identity to SEQ ID NO: 62; the heavy chain sequence has at least 91% sequence identity to SEQ ID NO: 61 and the light chain sequence has at least 91% sequence identity to SEQ ID NO: 62; the heavy chain sequence has at least 92% sequence identity to SEQ ID NO: 61 and the light chain sequence has at least 92% sequence identity to SEQ ID NO: 62; the heavy chain sequence has at least 93% sequence identity to SEQ ID NO: 61 and the light chain sequence has at least 93% sequence identity to SEQ ID NO: 62; the heavy chain sequence has at least 94% sequence identity to SEQ ID NO: 61 and the light chain sequence has at least 94% sequence identity to SEQ ID NO: 62; the heavy chain sequence has at least 95% sequence identity to SEQ ID NO: 61 and the light chain sequence has at least 95% sequence identity to SEQ ID NO: 62; the heavy chain sequence has at least 96% sequence identity to SEQ ID NO: 61 and the light chain sequence has at least 96% sequence identity to SEQ ID NO: 62; the heavy chain sequence has at least 97% sequence identity to SEQ ID NO: 61 and the light chain sequence has at least 97% sequence identity to SEQ ID NO: 62; the heavy chain sequence has at least 98% sequence identity to SEQ ID NO: 61 and the light chain sequence has at least 98% sequence identity to SEQ ID NO: 62; the heavy chain sequence has at least 99% sequence identity to SEQ ID NO: 61 and the light chain sequence has at least 99% sequence identity to SEQ ID NO: 62; or the heavy chain sequence comprises SEQ ID NO: 61 and the light chain sequence comprises SEQ ID NO: 62.

Yet further exemplary anti-PD-L1 antibodies that can be used in an anti-PD-L1/TGFβ Trap are described in US patent publication U.S. Pat. No. 7,943,743.

In one embodiment of the disclosure, the anti-PD-L1 antibody is MDX-1105.

In certain embodiments, the anti-PD-L1 antibody is MED-4736.

### Constant Region

The proteins and peptides of the disclosure can include a constant region of an immunoglobulin or a fragment, analog, variant, mutant, or derivative of the constant region. In certain embodiments, the constant region is derived from a human immunoglobulin heavy chain, for example, IgG1, IgG2, IgG3, IgG4, or other classes. In certain embodiments, the constant region includes a CH2 domain. In certain embodiments, the constant region includes CH2 and CH3 domains or includes hinge-CH2-CH3. Alternatively, the constant region can include all or a portion of the hinge region, the CH2 domain and/or the CH3 domain.

In one embodiment, the constant region contains a mutation that reduces affinity for an Fc receptor or reduces Fc effector function. For example, the constant region can contain a mutation that eliminates the glycosylation site within the constant region of an IgG heavy chain. In some embodiments, the constant region contains mutations, deletions, or insertions at an amino acid position corresponding to Leu234, Leu235, Gly236, Gly237, Asn297, or Pro331 of IgG1 (amino acids are numbered according to EU nomenclature). In a particular embodiment, the constant region contains a mutation at an amino acid position corresponding to Asn297 of IgG1. In alternative embodiments, the constant region contains mutations, deletions, or insertions at an amino acid position corresponding to Leu281, Leu282, Gly283, Gly284, Asn344, or Pro378 of IgG1.

In some embodiments, the constant region contains a CH2 domain derived from a human IgG2 or IgG4 heavy chain. Preferably, the CH2 domain contains a mutation that eliminates the glycosylation site within the CH2 domain. In one embodiment, the mutation alters the asparagine within the Gln-Phe-Asn-Ser (SEQ ID NO: 15) amino acid sequence within the CH2 domain of the IgG2 or IgG4 heavy chain. Preferably, the mutation changes the asparagine to a glutamine. Alternatively, the mutation alters both the phenylalanine and the asparagine within the Gln-Phe-Asn-Ser (SEQ ID NO: 15) amino acid sequence. In one embodiment, the Gln-Phe-Asn-Ser (SEQ ID NO: 15) amino acid sequence is replaced with a Gln-Ala-Gln-Ser (SEQ ID NO: 16) amino acid sequence. The asparagine within the Gln-Phe-Asn-Ser (SEQ ID NO: 15) amino acid sequence corresponds to Asn297 of IgG1.

In another embodiment, the constant region includes a CH2 domain and at least a portion of a hinge region. The hinge region can be derived from an immunoglobulin heavy chain, e.g., IgG1, IgG2, IgG3, IgG4, or other classes. Preferably, the hinge region is derived from human IgG1, IgG2, IgG3, IgG4, or other suitable classes. More preferably the hinge region is derived from a human IgG1 heavy chain. In one embodiment the cysteine in the Pro-Lys-Ser-Cys-Asp-Lys (SEQ ID NO: 17) amino acid sequence of the IgG1 hinge region is altered. In certain embodiments, the Pro-Lys-Ser-Cys-Asp-Lys (SEQ ID NO: 17) amino acid sequence is replaced with a Pro-Lys-Ser-Ser-Asp-Lys (SEQ ID NO: 18) amino acid sequence. In certain embodiments, the constant region includes a CH2 domain derived from a first antibody isotype and a hinge region derived from a second antibody isotype. In certain embodiments, the CH2 domain is derived from a human IgG2 or IgG4 heavy chain, while the hinge region is derived from an altered human IgG1 heavy chain.

The alteration of amino acids near the junction of the Fc portion and the non-Fc portion can dramatically increase the serum half-life of the Fc fusion protein (PCT publication WO 0158957, the disclosure of which is hereby incorporated by reference). Accordingly, the junction region of a protein or polypeptide of the present disclosure can contain alterations that, relative to the naturally-occurring sequences of an immunoglobulin heavy chain and erythropoietin, preferably lie within about 10 amino acids of the junction point. These amino acid changes can cause an increase in hydrophobicity. In one embodiment, the constant region is derived from an IgG sequence in which the C-terminal lysine residue is replaced. Preferably, the C-terminal lysine of an IgG sequence is replaced with a non-lysine amino acid, such as alanine or leucine, to further increase serum half-life. In another embodiment, the constant region is derived from an IgG sequence in which the Leu-Ser-Leu-Ser (SEQ ID NO: 19) amino acid sequence near the C-terminus of the constant region is altered to eliminate potential junctional T-cell epitopes. For example, in one embodiment, the Leu-Ser-Leu-Ser (SEQ ID NO: 19) amino acid sequence is replaced with an Ala-Thr-Ala-Thr (SEQ ID NO: 20) amino acid sequence. In other embodiments, the amino acids within the Leu-Ser-Leu-Ser (SEQ ID NO: 19) segment are replaced with other amino acids such as glycine or proline. Detailed methods of generating amino acid substitutions of the Leu-Ser-Leu-Ser (SEQ ID NO: 19) segment near the C-terminus of an IgG1, IgG2, IgG3, IgG4, or other immunoglobulin class molecule have been described in U.S. Patent Publication No. 20030166877, the disclosure of which is hereby incorporated by reference.

Suitable hinge regions for the present disclosure can be derived from IgG1, IgG2, IgG3, IgG4, and other immunoglobulin classes. The IgG1 hinge region has three cysteines, two of which are involved in disulfide bonds between the two heavy chains of the immunoglobulin. These same cysteines permit efficient and consistent disulfide bonding formation between Fc portions. Therefore, a hinge region of the present disclosure is derived from IgG1, e.g., human IgG1. In some embodiments, the first cysteine within the human IgG1 hinge region is mutated to another amino acid, preferably serine. The IgG2 isotype hinge region has four disulfide bonds that tend to promote oligomerization and possibly incorrect disulfide bonding during secretion in recombinant systems. A suitable hinge region can be derived from an IgG2 hinge; the first two cysteines are each preferably mutated to another amino acid. The hinge region of IgG4 is known to form interchain disulfide bonds inefficiently. However, a suitable hinge region for the present disclosure can be derived from the IgG4 hinge region, preferably containing a mutation that enhances correct formation of disulfide bonds between heavy chain-derived moieties (Angal S, et al. (1993) Mol. Immunol., 30:105-8).

In accordance with the present disclosure, the constant region can contain CH2 and/or CH3 domains and a hinge region that are derived from different antibody isotypes, e.g., a hybrid constant region. For example, in one embodiment, the constant region contains CH2 and/or CH3 domains derived from IgG2 or IgG4 and a mutant hinge region derived from IgG1. Alternatively, a mutant hinge region from another IgG subclass is used in a hybrid constant region. For example, a mutant form of the IgG4 hinge that allows efficient disulfide bonding between the two heavy chains can be used. A mutant hinge can also be derived from an IgG2 hinge in which the first two cysteines are each mutated to another amino acid. Assembly of such hybrid constant regions has been described in U.S. Patent Publication No. 20030044423, the disclosure of which is hereby incorporated by reference.

In accordance with the present disclosure, the constant region can contain one or more mutations described herein. The combinations of mutations in the Fc portion can have additive or synergistic effects on the prolonged serum half-life and increased in vivo potency of the bifunctional molecule. Thus, in one exemplary embodiment, the constant region can contain (i) a region derived from an IgG sequence in which the Leu-Ser-Leu-Ser (SEQ ID NO: 19) amino acid sequence is replaced with an Ala-Thr-Ala-Thr (SEQ ID NO: 20) amino acid sequence; (ii) a C-terminal alanine residue instead of lysine; (iii) a CH2 domain and a hinge region that are derived from different antibody isotypes, for example, an IgG2 CH2 domain and an altered IgG1 hinge region; and (iv) a mutation that eliminates the glycosylation site within the IgG2-derived CH2 domain, for example, a Gln-Ala-Gln-Ser (SEQ ID NO: 16) amino acid sequence instead of the Gln-Phe-Asn-Ser (SEQ ID NO: 15) amino acid sequence within the IgG2-derived CH2 domain.

### Antibody Fragments

The proteins and polypeptides of the disclosure can also include antigen-binding fragments of antibodies. Exemplary antibody fragments include scFv, Fv, Fab, F(ab′)₂, and single domain VHH fragments such as those of camelid origin.

Single-chain antibody fragments, also known as single-chain antibodies (scFvs), are recombinant polypeptides which typically bind antigens or receptors; these fragments contain at least one fragment of an antibody variable heavy-chain amino acid sequence (V_{H}) tethered to at least one fragment of an antibody variable light-chain sequence (V_{L}) with or without one or more interconnecting linkers. Such a linker may be a short, flexible peptide selected to assure that the proper three-dimensional folding of the V_{L} and V_{H} domains occurs once they are linked so as to maintain the target molecule binding-specificity of the whole antibody from which the single-chain antibody fragment is derived. Generally, the carboxyl terminus of the V_{L} or V_{H} sequence is covalently linked by such a peptide linker to the amino acid terminus of a complementary V_{L} and V_{H} sequence. Single-chain antibody fragments can be generated by molecular cloning, antibody phage display library or similar techniques. These proteins can be produced either in eukaryotic cells or prokaryotic cells, including bacteria.

Single-chain antibody fragments contain amino acid sequences having at least one of the variable regions or CDRs of the whole antibodies described in this specification, but are lacking some or all of the constant domains of those antibodies. These constant domains are not necessary for antigen binding, but constitute a major portion of the structure of whole antibodies. Single-chain antibody fragments may therefore overcome some of the problems associated with the use of antibodies containing part or all of a constant domain. For example, single-chain antibody fragments tend to be free of undesired interactions between biological molecules and the heavy-chain constant region, or other unwanted biological activity. Additionally, single-chain antibody fragments are considerably smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely than whole antibodies to provoke an immune response in a recipient.

Fragments of antibodies that have the same or comparable binding characteristics to those of the whole antibody may also be present. Such fragments may contain one or both Fab fragments or the F(ab′)₂ fragment. The antibody fragments may contain all six CDRs of the whole antibody, although fragments containing fewer than all of such regions, such as three, four or five CDRs, are also functional.

### Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In one aspect, the present disclosure provides an intravenous drug delivery formulation for use in a method of treating stage III NSCLC or inhibiting tumor growth in a treatment naïve cancer patient that includes 500 mg-2400 mg of a protein including a first polypeptide and a second polypeptide, the first polypeptide includes: (a) at least a variable region of a heavy chain of an antibody that binds to human protein Programmed Death Ligand 1 (PD-L1); and (b) human Transforming Growth Factor β Receptor TT (TGFβRII), or a fragment thereof, capable of binding Transforming Growth Factor (TGFβ), the second polypeptide includes at least a variable region of a light chain of an antibody that binds PD-L1, and the heavy chain of the first polypeptide and the light chain of the second polypeptide, when combined, form an antigen binding site that binds PD-L1.

In certain embodiments, a protein product of the present disclosure includes a first polypeptide that includes the amino acid sequence of SEQ ID NO: 3, and a second polypeptide that includes the amino acid sequence of SEQ ID NO: 1. In certain embodiments, a protein product of the present disclosure includes a first polypeptide that comprises the amino acid sequences of SEQ ID NOs: 35, 36, and 37, and a second polypeptide that comprises the amino acid sequences of SEQ ID NOs: 38, 39, and 40.

In certain embodiments of the present disclosure, the intravenous drug delivery formulation for use in a method of treating stage III NSCLC or inhibiting tumor growth in a treatment naïve cancer patient may include an about 500 mg to about 2400 mg dose (e.g., about 500 mg to about 2300 mg, about 500 mg to about 2200 mg, about 500 mg to about 2100 mg, about 500 mg to about 2000 mg, about 500 mg to about 1900 mg, about 500 mg to about 1800 mg, about 500 mg to about 1700 mg, about 500 mg to about 1600 mg, about 500 mg to about 1500 mg, about 500 mg to about 1400 mg, about 500 mg to about 1300 mg, about 500 mg to about 1200 mg, about 500 mg to about 1100 mg, about 500 mg to about 1000 mg, about 500 mg to about 900 mg, about 500 mg to about 800 mg, about 500 mg to about 700 mg, about 500 mg to about 600 mg, about 600 mg to 2400 mg, about 700 mg to 2400 mg, about 800 mg to 2400 mg, about 900 mg to 2400 mg, about 1000 mg to 2400 mg, about 1100 mg to 2400 mg, about 1200 mg to 2400 mg, about 1300 mg to 2400 mg, about 1400 mg to 2400 mg, about 1500 mg to 2400 mg, about 1600 mg to 2400 mg, about 1700 mg to 2400 mg, about 1800 mg to 2400 mg, about 1900 mg to 2400 mg, about 2000 mg to 2400 mg, about 2100 mg to 2400 mg, about 2200 mg to 2400 mg, or about 2300 mg to 2400 mg) of a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)). In certain embodiments, the intravenous drug delivery formulation may include an about 500 to about 2000 mg dose of a protein of the present disclosure (e.g., anti-PD-L/TGFβ Trap (e.g., including a first polypeptide that includes the amino acid sequence of SEQ ID NO: 3, and a second polypeptide that includes the amino acid sequence of SEQ ID NO: 1)). In certain embodiments, the intravenous drug delivery formulation may include an about 500 mg dose of a protein product of the present disclosure with a first polypeptide that includes the amino acid sequence of SEQ ID NO: 3, and a second polypeptide that includes the amino acid sequence of SEQ ID NO: 1. In certain embodiments, the intravenous drug delivery formulation may include a 500 mg dose of a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)). In certain embodiments, the intravenous drug delivery formulation may include an about 1200 mg dose of a protein product of the present disclosure with a first polypeptide that includes the amino acid sequence of SEQ ID NO: 3, and a second polypeptide that includes the amino acid sequence of SEQ ID NO: 1. In certain embodiments, the intravenous drug delivery formulation may include a 1200 mg dose of a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)). In certain embodiments, the intravenous drug delivery formulation may include an about 1800 mg dose of a protein product of the present disclosure with a first polypeptide that includes the amino acid sequence of SEQ ID NO: 3, and a second polypeptide that includes the amino acid sequence of SEQ ID NO: 1. In certain embodiments, the intravenous drug delivery formulation may include a 1800 mg dose of a protein of the present disclosure (e.g., anti-PD-L/TGFβ Trap (e.g., including a first polypeptide that includes the amino acid sequence of SEQ ID NO: 3, and a second polypeptide that includes the amino acid sequence of SEQ ID NO: 1)). In certain embodiments, the intravenous drug delivery formulation may include a 1800 mg dose of a protein of the present disclosure (e.g., anti-PD-L1/TGFβ Trap (e.g., including a first polypeptide comprising the amino acid sequences of SEQ ID NOs: 35, 36, and 37, and a second polypeptide comprising the amino acid sequences of SEQ ID NOs: 38, 39, and 40)). In certain embodiments, the intravenous drug delivery formulation may include an about 2400 mg dose of a protein product of the present disclosure with a first polypeptide that includes the amino acid sequence of SEQ ID NO: 3, and a second polypeptide that includes the amino acid sequence of SEQ ID NO: 1. In certain embodiments, the intravenous drug delivery formulation may include a 2400 mg dose of a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)). In certain embodiments, the intravenous drug delivery formulation may include a 2400 mg dose of a protein of the present disclosure (e.g., anti-PD-L1/TGFβ Trap (e.g., including a first polypeptide comprising the amino acid sequences of SEQ ID NOs: 35, 36, and 37, and a second polypeptide comprising the amino acid sequences of SEQ ID NOs: 38, 39, and 40)).

In certain embodiments, the intravenous drug delivery formulation for use in a method of treating stage III NSCLC or inhibiting tumor growth in a treatment naïve cancer patient may include an about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg) of a protein product of the present disclosure (e.g., anti-PD-L1/TGFβ Trap). In certain embodiments, the intravenous drug delivery formulation for use in a method of treating stage III NSCLC or inhibiting tumor growth in a treatment naïve cancer patient may include an about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg) of a protein product with a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40.

In certain embodiments, the intravenous drug delivery formulation for use in a method of treating stage III NSCLC or inhibiting tumor growth in a treatment naïve cancer patient may include about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, or about 2400 mg of the protein of the present disclosure (e.g., anti-PD-L1/TGFβ Trap comprising a first polypeptide that comprises the amino acid sequences of SEQ ID NOs: 35, 36, and 37, and a second polypeptide that comprises the amino acid sequences of SEQ ID NOs: 38, 39, and 40).

The intravenous drug delivery formulation of the present disclosure for use in a method of treating stage III NSCLC or inhibiting tumor growth in a treatment naïve cancer patient may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may freeze-dried (lyophilized) and contained in about 12-60 vials. In certain embodiments, the formulation may be freeze-dried and about 45 mg of the freeze-dried formulation may be contained in one vial. In certain embodiments, the about 40 mg-about 100 mg of freeze-dried formulation may be contained in one vial. In certain embodiments, freeze dried formulation from 12, 27, or 45 vials are combined to obtained a therapeutic dose of the protein in the intravenous drug formulation. In certain embodiments, the formulation may be a liquid formulation of a protein product with a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40, and stored as about 250 mg/vial to about 2000 mg/vial (e.g., about 250 mg/vial to about 2000 mg/vial, about 250 mg/vial to about 1900 mg/vial, about 250 mg/vial to about 1800 mg/vial, about 250 mg/vial to about 1700 mg/vial, about 250 mg/vial to about 1600 mg/vial, about 250 mg/vial to about 1500 mg/vial, about 250 mg/vial to about 1400 mg/vial, about 250 mg/vial to about 1300 mg/vial, about 250 mg/vial to about 1200 mg/vial, about 250 mg/vial to about 1100 mg/vial, about 250 mg/vial to about 1000 mg/vial, about 250 mg/vial to about 900 mg/vial, about 250 mg/vial to about 800 mg/vial, about 250 mg/vial to about 700 mg/vial, about 250 mg/vial to about 600 mg/vial, about 250 mg/vial to about 500 mg/vial, about 250 mg/vial to about 400 mg/vial, about 250 mg/vial to about 300 mg/vial, about 300 mg/vial to about 2000 mg/vial, about 400 mg/vial to about 2000 mg/vial, about 500 mg/vial to about 2000 mg/vial, about 600 mg/vial to about 2000 mg/vial, about 700 mg/vial to about 2000 mg/vial, about 800 mg/vial to about 2000 mg/vial, about 900 mg/vial to about 2000 mg/vial, about 1000 mg/vial to about 2000 mg/vial, about 1100 mg/vial to about 2000 mg/vial, about 1200 mg/vial to about 2000 mg/vial, about 1300 mg/vial to about 2000 mg/vial, about 1400 mg/vial to about 2000 mg/vial, about 1500 mg/vial to about 2000 mg/vial, about 1600 mg/vial to about 2000 mg/vial, about 1700 mg/vial to about 2000 mg/vial, about 1800 mg/vial to about 2000 mg/vial, or about 1900 mg/vial to about 2000 mg/vial). In certain embodiments, the formulation may be a liquid formulation and stored as about 600 mg/vial. In certain embodiments, the formulation may be a liquid formulation and stored as about 1200 mg/vial. In certain embodiments, the formulation may be a liquid formulation and stored as about 1800 mg/vial. In certain embodiments, the formulation may be a liquid formulation and stored as about 2400 mg/vial. In certain embodiments, the formulation may be a liquid formulation and stored as about 250 mg/vial.

This disclosure provides a liquid aqueous pharmaceutical formulation including a therapeutically effective amount of the protein of the present disclosure (e.g., anti-PD-L/TGFβ Trap) in a buffered solution forming a formulation for use in a method of treating stage III NSCLC or inhibiting tumor growth in a treatment naïve cancer patient.

These compositions for use in a method of treating stage III NSCLC or inhibiting tumor growth in a treatment naïve cancer patient may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides for use in a method of treating stage III NSCLC or inhibiting tumor growth in a treatment naïve cancer patient, a formulation with an extended shelf life including a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)), in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation for use in a method of treating stage III NSCLC or inhibiting tumor growth in a treatment naïve cancer patient is prepared including a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40) in a pH-buffered solution. The buffer of this invention may have a pH ranging from about 4 to about 8, e.g., from about 4 to about 8, from about 4.5 to about 8, from about 5 to about 8, from about 5.5 to about 8, from about 6 to about 8, from about 6.5 to about 8, from about 7 to about 8, from about 7.5 to about 8, from about 4 to about 7.5, from about 4.5 to about 7.5, from about 5 to about 7.5, from about 5.5 to about 7.5, from about 6 to about 7.5, from about 6.5 to about 7.5, from about 4 to about 7, from about 4.5 to about 7, from about 5 to about 7, from about 5.5 to about 7, from about 6 to about 7, from about 4 to about 6.5, from about 4.5 to about 6.5, from about 5 to about 6.5, from about 5.5 to about 6.5, from about 4 to about 6.0, from about 4.5 to about 6.0, from about 5 to about 6, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate and other organic acid buffers.

In certain embodiments, the formulation for use in a method of treating stage III NSCLC or inhibiting tumor growth in a treatment naïve cancer patient includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/ml of citric acid (e.g., 1.305 mg/ml), about 0.3 mg/ml of sodium citrate (e.g., 0.305 mg/ml), about 1.5 mg/ml of disodium phosphate dihydrate (e.g., 1.53 mg/ml), about 0.9 mg/ml of sodium dihydrogen phosphate dihydrate (e.g., 0.86), and about 6.2 mg/ml of sodium chloride (e.g., 6.165 mg/ml). In certain embodiments, the buffer system includes about 1-1.5 mg/ml of citric acid, about 0.25 to about 0.5 mg/ml of sodium citrate, about 1.25 to about 1.75 mg/ml of disodium phosphate dihydrate, about 0.7 to about 1.1 mg/ml of sodium dihydrogen phosphate dihydrate, and 6.0 to 6.4 mg/ml of sodium chloride. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also alter with respect to the molecular weight of the polyol. For example, a lower amount of a monosaccharide (e.g. mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/ml. In certain embodiments, the concentration of mannitol may be about 7.5 to about 15 mg/ml. In certain embodiments, the concentration of mannitol may be about 10- about 14 mg/ml. In certain embodiments, the concentration of mannitol may be about 12 mg/ml. In certain embodiments, the polyol sorbitol may be included in the formulation.

A detergent or surfactant may also be added to the formulation. Exemplary detergents include nonionic detergents such as polysorbates (e.g. polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hilfsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

### Lyophilized Formulation

The lyophilized formulation for use in a method of treating stage III NSCLC or inhibiting tumor growth in a treatment naïve cancer patient of the present disclosure includes the anti-PD-L1/TGFβ Trap molecule and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between about 6 to about 8. In certain embodiments, the pH range for the lyophilized drug product may be from about 7 to about 8.

In certain embodiments, a salt or buffer components may be added in an amount of about 10 mM-about 200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product for use in a method of treating stage III NSCLC or inhibiting tumor growth in a treatment naïve cancer patient may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g. phosphate-buffered saline), sterile saline solution, Ringer's solution or dextrose solution.

In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product of the instant disclosure is constituted to about 4.5 mL water for injection and diluted with 0.9% saline solution (sodium chloride solution).

### Liquid Formulation

In embodiments, the protein product of the present disclosure is formulated as a liquid formulation for use in a method of treating stage III NSCLC or inhibiting tumor growth in a treatment naïve cancer patient. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments vials may be filled with about 61.2 mL of the protein product solution in order to allow an extractable volume of 60 mL. In certain embodiments, the liquid formulation may be diluted with 0.9% saline solution. In certain embodiments vials may contain about 61.2 mL of the protein product (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)) solution of about 20 mg/mL to about 50 mg/mL (e.g., about 20 mg/mL, about 25 mg/mL, about 30 mg/mL, about 35 mg/mL, about 40 mg/mL, about 45 mg/mL or about 50 mg/mL) in order to allow an extractable volume of 60 mL for delivering about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg) of the protein product (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40)) to a subject.

In certain embodiments, vials may contain about 61.2 mL of the protein product solution (protein product with a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40) of about 20 mg/mL to about 50 mg/mL (e.g., about 20 mg/mL, about 25 mg/mL, about 30 mg/mL, about 35 mg/mL, about 40 mg/mL, about 45 mg/mL or about 50 mg/mL) in order to allow an extractable volume of 60 mL for delivering about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg) of the protein product to a treatment naïve subject.

In certain embodiments, the liquid formulation for use in a method of treating stage III NSCLC or inhibiting tumor growth in a treatment naïve cancer patient, while minimizing the development of pathological conditions (e.g., pulmonary fibrosis, pneumonitis) associated with concomitant radiotherapy, and increasing the time-to-onset of metastasis and/or time to distant metastasis of the stage III NSCLC in the patient, of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variant of peptides and proteins that may occur during fermentation, harvest/cell clarification, purification, drug substance/drug product storage and during sample analysis. Deamidation is the loss of NH₃ from a protein forming a succinimide intermediate that can undergo hydrolysis. The succinimide intermediate results in a 17 u mass decrease of the parent peptide. The subsequent hydrolysis results in an 18 u mass increase. Isolation of the succinimide intermediate is difficult due to instability under aqueous conditions. As such, deamidation is typically detectable as 1 u mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, the liquid formulation for use in a method of treating stage III NSCLC or inhibiting tumor growth in a treatment naïve cancer patient of the present disclosure may be preserved under conditions of pH and humidity to prevent deamidation of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g. phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (TV) formulations may be the preferred administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

A preservative may be optionally added to the formulations herein to reduce bacterial action. The addition of a preservative may, for example, facilitate the production of a multi-use (multiple-dose) formulation.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g. phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

### Method of Treating Cancer or Inhibiting Tumor Growth

In one aspect the present disclosure provides a method of treating stage III NSCLC or inhibiting tumor growth in a treatment naïve subject in need thereof, while minimizing the development of pathological conditions (e.g., pulmonary fibrosis, pneumonitis) associated with concomitant radiotherapy, and increasing the time-to-onset of metastasis and/or time to distant metastasis of the stage III NSCLC in the patient, the method including administering to the subject a dose of at least 500 mg of a protein including a first polypeptide and a second polypeptide. The first polypeptide includes: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ). The second polypeptide includes at least a variable region of a light chain of an antibody that binds PD-L1, and the heavy chain of the first polypeptide and the light chain of the second polypeptide, when combined, form an antigen binding site that binds PD-L1.

In one aspect, the present disclosure provides a method of treating advanced unresectable stage III non-small cell lung cancer (NSCLC) in a patient by administering to the patient an anti-PD-L1/TGFβ Trap in combination with cCRT (e.g., platinum-based chemoradiation) followed by administering the anti-PD-L1/TGFβ Trap to the patient. In certain embodiments, the present disclosure provides a method of treating advanced unresectable stage 111 NSCLC in a patient by administering to the patient an anti-PD-L1/TGFβ Trap in combination with and following concurrent platinum-based chemoradiation (cCRT).

In certain embodiments, patients treated with cisplatin/pemetrexed and radiation therapy (cCRT) in combination with anti-PD-L1/TGFβ Trap are diagnosed with advanced unresectable stage III NSCLC, which exhibits non-squamous histology.

In certain embodiment, cCRT is administered as either cisplatin/etoposide, cisplatin/pemetrexed, or carboplatin/paclitaxel concurrently with 60-66 Gy (e.g., 60 Gy) total dose of radiation delivered by intensity-modulated radiation therapy. In certain embodiment, cCRT is administered as cisplatin/etoposide concurrently with 60-66 Gy (e.g., 60 Gy) total dose of radiation delivered by intensity-modulated radiation therapy. In certain embodiment, cCRT is administered as carboplatin/paclitaxel concurrently with 60-66 Gy (e.g., 60 Gy) total dose of radiation delivered by intensity-modulated radiation therapy. In certain embodiment, cCRT is administered as cisplatin/pemetrexed concurrently with 60-66 Gy (e.g., 60 Gy) total dose of radiation delivered by intensity-modulated radiation therapy.

In certain embodiments, the method of treating stage III NSCLC or inhibiting tumor growth of the present disclosure involves administering to a treatment naïve subject a protein including two peptides in which the first polypeptide includes the amino acid sequence of SEQ ID NO: 3, and the second polypeptide includes the amino acid sequence of SEQ ID NO: 1. In certain embodiments, the protein is an anti-PD-L1/TGFβ Trap molecule.

In an embodiment, the treatment naïve subject treated in accordance with the methods disclosed herein has not received prior therapy with the bifunctional protein of the present disclosure (anti-PD-L1/TGFβ Trap molecule). In some embodiments, the treatment naïve cancer patient to be treated in accordance with the methods of the present disclosure has or does not have epidermal growth factor receptor (EGFR) sensitizing (activating) mutation, anaplastic lymphoma kinase (ALK) translocation, and/or ROS1 mutation.

In certain embodiments, the method of treating stage III NSCLC or inhibiting tumor growth, while minimizing the development of pathological conditions (e.g., pulmonary fibrosis, pneumonitis) associated with concomitant radiotherapy, and increasing the time-to-onset of metastasis and/or time to distant metastasis of the stage III NSCLC in the patient, of the present disclosure involves administering to a treatment naïve subject a protein (e.g., an anti-PD-L1/TGFβ Trap molecule (e.g., including a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40)) at a dose of about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg). In certain embodiments, about 1200 mg of anti-PD-L1/TGFβ Trap molecule is administered to a treatment naïve stage III NSCLC subject (e.g., an unresectable stage III NSCLC subject) once every two weeks. In certain embodiments, about 1800 mg of anti-PD-L1/TGFβ Trap molecule is administered to a treatment naïve stage III NSCLC subject (e.g., an unresectable stage III NSCLC subject) once every three weeks. In certain embodiments, about 1200 mg of a protein product with a first polypeptide that includes the amino acid sequence of SEQ ID NO: 3 and a second polypeptide that includes the amino acid sequence of SEQ ID NO: 1 is administered to a treatment naïve subject once every two weeks. In certain embodiments, about 1800 mg of a protein product with a first polypeptide that includes the amino acid sequence of SEQ ID NO: 3 and a second polypeptide that includes the amino acid sequence of SEQ ID NO: 1 is administered to a treatment naïve stage III NSCLC subject (e.g., an unresectable stage III NSCLC subject) once every three weeks. In certain embodiments, about 1800 mg of a protein product with a first polypeptide that comprises the amino acid sequences of SEQ ID NOs: 35, 36, and 37, and a second polypeptide that comprises the amino acid sequences of SEQ ID NOs: 38, 39, and 40 is administered to a treatment naïve stage III NSCLC subject (e.g., an unresectable stage III NSCLC subject) once every three weeks. In certain embodiments, about 2400 mg of a protein product with a first polypeptide that includes the amino acid sequence of SEQ ID NO: 3 and a second polypeptide that includes the amino acid sequence of SEQ ID NO: 1 is administered to a subject once every three weeks. In certain embodiments, about 2400 mg of a protein product with a first polypeptide that comprises the amino acid sequences of SEQ ID NOs: 35, 36, and 37, and a second polypeptide that comprises the amino acid sequences of SEQ ID NOs: 38, 39, and 40 is administered to a subject once every three weeks.

In certain embodiments, the dose administered to a treatment naïve stage III NSCLC subject (e.g., an unresectable stage III NSCLC subject) may be about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, 1875 mg, about 1900 mg, about 1925 mg, about 1950 mg, about 1975 mg, about 2000 mg, about 2025 mg, about 2050 mg, about 2075 mg, 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, or about 2400 mg.

In certain embodiments, the dose administered to a treatment naïve stage III NSCLC subject (e.g., an unresectable stage III NSCLC subject) may be administered once every two weeks. In certain embodiments, the dose administered to a treatment naïve stage III NSCLC subject (e.g., an unresectable stage III NSCLC subject) may be administered once every three weeks. In certain embodiments, the protein may be administered by intravenous administration, e.g., with a prefilled bag, a prefilled pen, or a prefilled syringes. In certain embodiments, the protein is administered intravenously from a 250 ml saline bag, and the intravenous infusion may be for about one hour (e.g., 50 to 80 minutes). In certain embodiments, the bag is connected to a channel comprising a tube and/or a needle.

In some embodiments, the stage III NSCLC exhibits squamous or non-squamous histology. For example, in an embodiment, the method treats squamous stage III NSCLC. In some embodiments, the method treats non-squamous stage III NSCLC.

In certain embodiments, treatment naïve subjects or patients with stage III NSCLC (e.g., squamous or non-squamous stage III NSCLC) are treated by intravenously administering at least 500 mg (e.g., about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or more) of anti-PD-L1/TGFβ Trap, which includes a first polypeptide that includes the amino acid sequence of SEQ ID NO: 3, and a second polypeptide that includes the amino acid sequence of SEQ ID NO: 1. In certain embodiments, treatment naïve subjects or patients with stage III NSCLC (e.g., squamous or non-squamous stage III NSCLC) are treated by intravenously administering at least 500 mg (e.g., about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or more) of anti-PD-L1/TGFβ Trap, which includes a first polypeptide that comprises the amino acid sequences of SEQ ID NOs: 35, 36, and 37, and a second polypeptide that comprises the amino acid sequences of SEQ ID NOs: 38, 39, and 40. In certain embodiments, treatment naïve subjects or patients with stage III NSCLC (e.g., squamous or non-squamous stage III NSCLC) are treated by intravenously administering 2400 mg of anti-PD-L1/TGFβ Trap, which includes a first polypeptide that comprises the amino acid sequences of SEQ ID NOs: 35, 36, and 37, and a second polypeptide that comprises the amino acid sequences of SEQ ID NOs: 38, 39, and 40.

In certain embodiments, treatment naïve subjects or patients with stage III NSCLC (e.g., squamous or non-squamous stage III NSCLC) are treated by intravenously administering about 1200 mg-about 2400 mg (e.g., about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 2400 mg, about 1400 mg to about 2400 mg, about 1500 mg to about 2400 mg, about 1600 mg to about 2400 mg, about 1700 mg to about 2400 mg, about 1800 mg to about 2400 mg, about 1900 mg to about 2400 mg, about 2000 mg to about 2400 mg, about 2100 mg to about 2400 mg, about 2200 mg to about 2400 mg, or about 2300 mg to about 2400 mg) of anti-PD-L1/TGFβ Trap, which includes a first polypeptide that includes the amino acid sequence of SEQ ID NO: 3, and a second polypeptide that includes the amino acid sequence of SEQ ID NO: 1.

In certain embodiments, treatment naïve subjects or patients with stage III NSCLC (e.g., squamous or non-squamous stage III NSCLC) are treated by intravenously administering about 1200 mg-about 2400 mg (e.g., about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 2400 mg, about 1400 mg to about 2400 mg, about 1500 mg to about 2400 mg, about 1600 mg to about 2400 mg, about 1700 mg to about 2400 mg, about 1800 mg to about 2400 mg, about 1900 mg to about 2400 mg, about 2000 mg to about 2400 mg, about 2100 mg to about 2400 mg, about 2200 mg to about 2400 mg, or about 2300 mg to about 2400 mg) of anti-PD-L1/TGFβ Trap, which includes a first polypeptide that comprises the amino acid sequences of SEQ ID NOs: 35, 36, and 37, and a second polypeptide that comprises the amino acid sequences of SEQ ID NOs: 38, 39, and 40.

In some embodiments, treatment naïve subjects or patients with stage III NSCLC (e.g., squamous or non-squamous stage III NSCLC) are treated by intravenously administering anti-PD-L1/TGFβ Trap at a dose of about 1200 mg once every 2 weeks. In some embodiments, treatment naïve subjects or patients with stage III NSCLC (e.g., squamous or non-squamous stage III NSCLC) are treated by intravenously administering anti-PD-L1/TGFβ Trap at a dose of about 1800 mg once every 3 weeks. In some embodiments, treatment naïve subjects or patients with stage III NSCLC (e.g., squamous or non-squamous stage III NSCLC) are treated by intravenously administering anti-PD-L1/TGFβ Trap at a dose of about 2400 mg once every 3 weeks. In some embodiments, treatment naïve subjects or patients with stage III NSCLC (e.g., squamous or non-squamous stage III NSCLC) are treated by intravenously administering anti-PD-L1/TGFβ Trap at a dose of 2400 mg once every 3 weeks.

In certain embodiments, the stage III NSCLC to be treated is PD-L1 positive. In certain embodiments, the stage III NSCLC to be be treated is PD-L1 negative. In exemplary embodiments, the stage III NSCLC to be treated exhibits high PD-L1 expression (e.g., “high PD-L1”). In exemplary embodiments, the stage III NSCLC to be treated does not exhibit PD-L expression. In exemplary embodiments, patients with stage III NSCLC to be treated are diagnosed with PD-L1 positive stage III NSCLC. In exemplary embodiments, patient with stage III NSCLC to be treated are diagnosed with PD-L1 negative stage III NSCLC.

Methods of detecting a biomarker, such as PD-L1 for example, on a cancer or tumor, are routine in the art and are contemplated herein. Non-limiting examples include immunohistochemistry, immunofluorescence and fluorescence activated cell sorting (FACS). In certain embodiments, the PD-L1 expression levels in the stage III NSCLC is detected using an anti-PD-L1 antibody. The tissue sample may be a formalin-fixed, paraffin-embedded stage III NSCLC tissue.

In some embodiments, treatment naïve subjects or patients with PD-L1 high, stage III NSCLC or irrespective of PD-L1 expression (stage III NSCLC is either PD-L1 positive or PD-L1 negative) (e.g., squamous or non-squamous stage III NSCLC) are treated by intravenously administering anti-PD-L1/TGFβ Trap at a dose of at least 500 mg. In some embodiments, treatment naïve subjects or patients with PD-L1 high, stage III NSCLC or irrespective of PD-L1 expression (stage III NSCLC is either PD-L1 positive or PD-L1 negative) (e.g., squamous or non-squamous stage III NSCLC) are treated by intravenously administering anti-PD-L1/TGFβ Trap at a dose of about 1200 mg once every 2 weeks. In some embodiments, treatment naïve subjects or patients with PD-L1 high, stage III NSCLC or irrespective of PD-L1 expression (stage III NSCLC is either PD-L1 positive or PD-L1 negative) (e.g., squamous or non-squamous stage III NSCLC) are treated by intravenously administering anti-PD-L1/TGFβ Trap at a dose of about 1800 mg once every 3 weeks. In some embodiments, treatment naïve subjects or patients with PD-L1 high, stage III NSCLC or irrespective of PD-L1 expression (stage III NSCLC is either PD-L1 positive or PD-L1 negative) (e.g., squamous or non-squamous stage III NSCLC) are treated by intravenously administering anti-PD-L1/TGFβ Trap at a dose of about 2400 mg once every 3 weeks.

In certain embodiments, patients treated with cisplatin/pemetrexed and radiation therapy (cCRT) in combination with anti-PD-L1/TGFβ Trap are diagnosed with advanced unresectable stage III NSCLC, which expresses PD-L1. In certain embodiments, patients treated with cisplatin/pemetrexed and radiation therapy (cCRT) in combination with anti-PD-L1/TGFβ Trap are diagnosed with advanced unresectable stage III NSCLC, which does not express PD-L1. In certain embodiments, patients diagnosed with advanced unresectable stage III NSCLC are treated with chemotherapy (e.g., cisplatin/pemetrexed) and radiation therapy (cCRT) in combination with anti-PD-L1/TGFβ Trap, irrespective of PD-L1 expression (stage III NSCLC is either PD-L1 positive or PD-L1 negative).

In some embodiments, patients diagnosed with advanced stage III NSCLC (e.g., squamous or non-squamous stage III NSCLC) are treated with chemotherapy (e.g., combination of cisplatin and etoposide, or combination of carboplatin and paclitaxel) and radiation therapy (cCRT) in combination with anti-PD-L1/TGFβ Trap, irrespective of PD-L1 expression (stage III NSCLC is either PD-L1 positive or PD-L1 negative), by intravenously administering anti-PD-L1/TGFβ Trap at a dose of at least 500 mg. In some embodiments, patients diagnosed with advanced stage III NSCLC (e.g., squamous or non-squamous stage III NSCLC) are treated with chemotherapy (e.g., combination of cisplatin and etoposide, or combination of carboplatin and paclitaxel) and radiation therapy (cCRT) in combination with anti-PD-L1/TGFβ Trap, irrespective of PD-L1 expression (stage III NSCLC is either PD-L1 positive or PD-L negative), by intravenously administering anti-PD-L/TGFβ Trap at a dose of about 1200 mg once every 2 weeks. In some embodiments, patients diagnosed with advanced stage III NSCLC (e.g., squamous or non-squamous stage III NSCLC) are treated with chemotherapy (e.g., combination of cisplatin and etoposide, or combination of carboplatin and paclitaxel) and radiation therapy (cCRT) in combination with anti-PD-L1/TGFβ Trap, irrespective of PD-L1 expression (stage III NSCLC is either PD-L1 positive or PD-L1 negative), by intravenously administering anti-PD-L1/TGFβ Trap at a dose of about 1800 mg or 2400 mg once every 3 weeks.

In some embodiments, patients diagnosed with advanced unresectable stage III NSCLC, with a non-squamous histology, are treated with chemotherapy (e.g., combination of cisplatin and pemetrexed) and radiation therapy (cCRT) in combination with anti-PD-L1/TGFβ Trap, irrespective of PD-L1 expression (stage III NSCLC is either PD-L1 positive or PD-L1 negative), by intravenously administering anti-PD-L1/TGFβ Trap at a dose of at least 500 mg. In some embodiments, patients diagnosed with advanced unresectable stage III NSCLC, with a non-squamous histology, are treated with chemotherapy (e.g., combination of cisplatin and pemetrexed) and radiation therapy (cCRT) in combination with anti-PD-L1/TGFβ Trap, irrespective of PD-L1 expression (stage III NSCLC is either PD-L1 positive or PD-L negative), by intravenously administering anti-PD-L1/TGFβ Trap at a dose of about 1200 mg once every 2 weeks. In some embodiments, patients diagnosed with advanced unresectable stage III NSCLC, with a non-squamous histology, are treated with chemotherapy (e.g., combination of cisplatin and pemetrexed) and radiation therapy (cCRT) in combination with anti-PD-L1/TGFβ Trap, irrespective of PD-L1 expression (stage III NSCLC is either PD-L1 positive or PD-L negative), by intravenously administering anti-PD-L1/TGFβ Trap at a dose of about 1800 mg or 2400 mg once every 3 weeks.

In certain embodiments, the present disclosure provides a method of treating advanced unresectable stage III NSCLC in a patient by administering to the patient an anti-PD-L/TGFβ Trap at a dose of at least 500 mg (e.g., about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or more) in combination with and following concurrent platinum-based chemoradiation (cCRT). In certain embodiments, the present disclosure provides a method of treating advanced unresectable stage III NSCLC in a patient by administering to the patient an anti-PD-L1/TGFβ Trap at a dose of about 1200 mg in combination with and following concurrent platinum-based chemoradiation (cCRT). In certain embodiments, the present disclosure provides a method of treating advanced unresectable stage III NSCLC in a patient by administering to the patient an anti-PD-L1/TGFβ Trap at a dose of about 1800 mg in combination with and following concurrent platinum-based chemoradiation (cCRT). In certain embodiments, the present disclosure provides a method of treating advanced unresectable stage III NSCLC in a patient by administering to the patient an anti-PD-L/TGFβ Trap at a dose of about 2400 mg in combination with and following concurrent platinum-based chemoradiation (cCRT).

In some embodiments, the treatment naïve subject or patient to be treated has a mutation selected from EGFR sensitizing mutation, ALK translocation, and ROS1 mutation. For example, in some embodiments, treatment naïve subjects or patients with PD-L1 high, stage III NSCLC or irrespective of PD-L1 expression (stage III NSCLC is either PD-L1 positive or PD-L1 negative) (e.g., squamous or non-squamous stage III NSCLC) who have a mutation selected from EGFR sensitizing mutation, ALK translocation, and ROS1 mutation, are treated by intravenously administering anti-PD-L1/TGFβ Trap at a dose of at least 500 mg (e.g., about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or more). In some embodiments, treatment naïve subjects or patients with PD-L1 high, stage 111 NSCLC or irrespective of PD-L1 expression (stage 111 NSCLC is either PD-L1 positive or PD-L1 negative) (e.g., squamous or non-squamous stage III NSCLC) who have a mutation selected from EGFR sensitizing mutation, ALK translocation, and ROS1 mutation mutation are treated by intravenously administering anti-PD-L1/TGFβ Trap at a dose of about 1200 mg once every 2 weeks. In some embodiments, treatment naïve subjects or patients with PD-L1 high, stage III NSCLC or irrespective of PD-L1 expression (stage III NSCLC is either PD-L1 positive or PD-L1 negative) (e.g., squamous or non-squamous NSCLC) who have a mutation selected from EGFR sensitizing mutation, ALK translocation, and ROS1 mutation mutation are treated by intravenously administering anti-PD-L1/TGFβ Trap at a dose of about 1800 mg once every 3 weeks. In some embodiments, treatment naïve subjects or patients with PD-L1 high, stage III NSCLC or irrespective of PD-L1 expression (stage III NSCLC is either PD-L1 positive or PD-L1 negative) (e.g., squamous or non-squamous NSCLC) who have a mutation selected from EGFR sensitizing mutation, ALK translocation, and ROS1 mutation mutation are treated by intravenously administering anti-PD-L1/TGFβ Trap at a dose of about 2400 mg once every 3 weeks.

In some embodiments, the treatment naïve subject or patient to be treated does not have a mutation selected from EGFR sensitizing mutation, ALK translocation, ROS1 mutation, and BRAF V600E mutation. For example, in some embodiments, treatment naïve subjects or patients with PD-L1 high, stage III NSCLC or irrespective of PD-L1 expression (stage III NSCLC is either PD-L1 positive or PD-L1 negative) (e.g., squamous or non-squamous stage III NSCLC) who do not have a mutation selected from EGFR sensitizing mutation, ALK translocation, ROS1 mutation, and BRAF V600E mutation, are treated by intravenously administering anti-PD-L1/TGFβ Trap at a dose of at least 500 mg (e.g., about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or more). In some embodiments, treatment naïve subjects or patients with PD-1 high, stage III NSCLC or irrespective of PD-L1 expression (stage III NSCLC is either PD-L1 positive or PD-1 negative) (e.g., squamous or non-squamous stage III NSCLC) who do not have a mutation selected from EGFR sensitizing mutation, ALK translocation, ROS1 mutation, and BRAF V600E mutation are treated by intravenously administering anti-PD-L1/TGFβ Trap at a dose of about 1200 mg once every 2 weeks. In some embodiments, treatment naïve subjects or patients with PD-1 high, stage III NSCLC or irrespective of PD-L1 expression (stage 111 NSCLC is either PD-1 positive or PD-L1 negative) (e.g., squamous or non-squamous NSCLC) who do not have a mutation selected from EGFR sensitizing mutation, ALK translocation, ROS1 mutation, and BRAF V600E mutation are treated by intravenously administering anti-PD-L1/TGFβ Trap at a dose of about 1800 mg once every 3 weeks. In some embodiments, treatment naïve subjects or patients with PD-1 high, stage III NSCLC or irrespective of PD-L expression (stage III NSCLC is either PD-1 positive or PD-L1 negative) (e.g., squamous or non-squamous NSCLC) who do not have a mutation selected from EGFR sensitizing mutation, ALK translocation, ROS1 mutation, and BRAF V600E mutation are treated by intravenously administering anti-PD-L1/TGFβ Trap at a dose of about 2400 mg once every 3 weeks.

In some embodiments, the methods of treatment disclosed herein result in a disease response or improved survival (e.g., survival of up to and including 6 months, 12 months, 18 months, 22 months, 28 months, 32 months, 38 months, 44 months, 50 months, 56 months, 62 months, 68 months, 74 months, 80 months, 86 months, 92 months, 98 months, 104 months, or 110 months) of the subject or patient. In certain embodiments, improved survival is at least 108 months. In some embodiments for example, the disease response may be a complete response, a partial response, or a stable disease. In some embodiments for example, the improved survival (e.g., survival of up to and including 6 months, 12 months, 18 months, 22 months, 28 months, 32 months, 38 months, 44 months, 50 months, 56 months, 62 months, 68 months, 74 months, 80 months, 86 months, 92 months, 98 months, 104 months, or 110 months) could be progression-free survival (PFS) or overall survival (OS). In certain embodiments, improved survival of PFS and/or OS is at least 108 months. In some embodiments, improvement (e.g., in PFS) is determined relative to a period prior to initiation of treatment with an anti-PD-L1/TGFβ Trap of the present disclosure. Methods of determining disease response (e.g, complete response, partial response, or stable disease) and patient survival (e.g, PFS, overall survival (e.g., survival of up to and including 6 months, 12 months, 18 months, 22 months, 28 months, 32 months, 38 months, 44 months, 50 months, 56 months, 62 months, 68 months, 74 months, 80 months, 86 months, 92 months, 98 months, 104 months, or 110 months)) for cancer or tumor therapy are routine in the art and are contemplated herein. In certain embodiments, patient survival is at least 108 months. In some embodiments, disease response is evaluated according to RECIST 1.1 after subjecting the treated patient to contrast-enhanced computed tomography (CT) or magnetic resonance imaging (MRI) of the affected area (e.g., chest/abdomen and pelvis covering the area from the superior extent of the thoracic inlet to the symphysis pubis).

### Delivery Device

In one aspect, the present disclosure provides a drug delivery device for use in a method of treating stage III NSCLC or inhibiting tumor growth in a treatment naïve cancer patient, wherein the device includes a formulation comprising about 500 mg-about 3000 mg of a protein including a first polypeptide and a second polypeptide, the first polypeptide includes: (a) at least a variable region of a heavy chain of an antibody that binds to human protein Programmed Death Ligand 1 (PD-L1); and (b) human Transforming Growth Factor Receptor II (TGFβRII), or a fragment thereof, capable of binding Transforming Growth Factor (TGFβ), the second polypeptide includes at least a variable region of a light chain of an antibody that binds PD-L1, and the heavy chain of the first polypeptide and the light chain of the second polypeptide, when combined, form an antigen binding site that binds PD-L1.

In certain embodiments, the device may be a bag, a pen, or a syringe. In certain embodiments, the bag may be connected to a channel comprising a tube and/or a needle.

In certain embodiments of the present disclosure, the drug delivery device for use in a method of treating stage III NSCLC or inhibiting tumor growth in a treatment naïve cancer patient, while minimizing the development of pathological conditions (e.g., pulmonary fibrosis, pneumonitis) associated with concomitant radiotherapy, and increasing the time-to-onset of metastasis and/or time to distant metastasis of the stage III NSCLC in the patient, may include an about 500 mg to about 3000 mg (e.g., about 500 mg to about 3000 mg, about 500 mg to about 2900 mg, about 500 mg to about 2800 mg, about 500 mg to about 2700 mg, about 500 mg to about 2600 mg, about 500 mg to about 2500 mg, about 500 mg to about 2400 mg, about 500 mg to about 2300 mg, about 500 mg to about 2200 mg, about 500 mg to about 2100 mg, about 500 mg to about 2000 mg, about 500 mg to about 1900 mg, about 500 mg to about 1800 mg, about 500 mg to about 1700 mg, about 500 mg to about 1600 mg, about 500 mg to about 1500 mg, about 500 mg to about 1400 mg, about 500 mg to about 1300 mg, about 500 mg to about 1200 mg, about 500 mg to about 1100 mg, about 500 mg to about 1000 mg, about 500 mg to about 900 mg, about 500 mg to about 800 mg, about 500 mg to about 700 mg, about 500 mg to about 600 mg, about 600 mg to about 3000 mg, about 700 mg to about 3000 mg, about 800 mg to about 3000 mg, about 900 mg to about 3000 mg, about 1000 mg to about 3000 mg, about 1100 mg to about 3000 mg, about 1200 mg to about 3000 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, or about 2900 mg to about 3000 mg) of a protein of the present disclosure (e.g., anti-PD-L1/TGFβ Trap, which includes a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40). In certain embodiments, the drug delivery device may include about 500 to about 1200 mg dose of a protein of the present disclosure (e.g., anti-PD-L1/TGFβ Trap, which includes a first polypeptide that includes the amino acid sequence of SEQ ID NO: 3, and a second polypeptide that includes the amino acid sequence of SEQ ID NO: 1). In certain embodiments, the drug delivery device may include about 500 mg dose of the protein of the present disclosure (e.g., anti-PD-L1/TGFβ Trap, which includes a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40).

In certain embodiments, the drug delivery device includes an about 1200 mg dose of a protein of the present disclosure (e.g., anti-PD-L1/TGFβ Trap, which includes a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40). In certain embodiments, the drug delivery device for use in a method of treating stage III NSCLC or inhibiting tumor growth in a treatment naïve cancer patient, while minimizing the development of pathological conditions (e.g., pulmonary fibrosis, pneumonitis) associated with concomitant radiotherapy, and increasing the time-to-onset of metastasis and/or time to distant metastasis of the stage III NSCLC in the patient, includes an about 1800 mg dose of a protein of the present disclosure (e.g., anti-PD-L1/TGFβ Trap, which includes a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40). In certain embodiments, the drug delivery device for use in a method of treating stage III NSCLC or inhibiting tumor growth in a treatment naïve cancer patient, while minimizing the development of pathological conditions (e.g., pulmonary fibrosis, pneumonitis) associated with concomitant radiotherapy, and increasing the time-to-onset of metastasis and/or time to distant metastasis of the stage III NSCLC in the patient, includes an about 1200 mg, about 1800 mg, or 2400 mg dose of the protein product with a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40.

In certain embodiments, the drug delivery device for use in a method of treating stage III NSCLC or inhibiting tumor growth in a treatment naïve cancer patient, while minimizing the development of pathological conditions (e.g., pulmonary fibrosis, pneumonitis) associated with concomitant radiotherapy, and increasing the time-to-onset of metastasis and/or time to distant metastasis of the stage III NSCLC in the patient, includes an about 1200 mg dose of the protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40)). In certain embodiments, the drug delivery device for use in a method of treating stage III NSCLC or inhibiting tumor growth in a treatment naïve cancer patient includes an about 1800 mg dose of the protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1; or a protein product with a first polypeptide that comprises the amino acid sequences of SEQ ID NOs: 35, 36, and 37, and a second polypeptide that comprises the amino acid sequences of SEQ ID NOs: 38, 39, and 40)). In certain embodiments, the drug delivery device for use in a method of treating stage III NSCLC or inhibiting tumor growth in a treatment naïve cancer patient may include about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, or about 2400 mg of the protein of the present disclosure (e.g., anti-PD-L1/TGFβ Trap, e.g., a protein product with a first polypeptide that comprises the amino acid sequences of SEQ ID NOs: 35, 36, and 37, and a second polypeptide that comprises the amino acid sequences of SEQ ID NOs: 38, 39, and 40).

### Protein Production

The antibody-cytokine Trap proteins are generally produced recombinantly, using mammalian cells containing a nucleic acid engineered to express the protein. Although one example of a suitable cell line and protein production method is described in Examples 1 and 2 of US 20150225483 A1, a wide variety of suitable vectors, cell lines and protein production methods have been used to produce antibody-based biopharmaceuticals and could be used in the synthesis of these antibody-cytokine Trap proteins.

### Therapeutic Indications

The anti-PD-L1/TGFβ Trap proteins described in the application (e.g., including a first polypeptide that includes the amino acid sequence of SEQ ID NO: 3, and a second polypeptide that includes the amino acid sequence of SEQ ID NO: 1), as well as the disclosed intravenous drug delivery formulations and delivery devices comprising said anti-PD-L/TGFβ Trap proteins, can be used to treat stage III NSCLC or reduce tumor growth in a treatment naïve patient.

The stage III NSCLC or tumor to be treated with an anti-PD-L1/TGFβ Trap may have elevated expression of PD-L1 and/or TGFβ in the tumor, the correlation of their expression levels with prognosis or disease progression, and preclinical and clinical experience on the sensitivity of the tumor to treatments targeting PD-L and TGFβ.

In some embodiments, the treatment naïve cancer patient to be treated in accordance with the methods of the present disclosure has or does not have a mutation selected from epidermal growth factor receptor (EGFR) sensitizing (activating) mutation, anaplastic lymphoma kinase (ALK) translocation, and ROS1 mutation. In some embodiments, the treatment naïve cancer (e.g., advanced stage III NSCLC (e.g., squamous or non-squamous stage III NSCLC) with high PD-L1 expression; PD-L1 positive advanced stage III NSCLC (e.g., squamous or non-squamous stage III NSCLC); or PD-L1 negative advanced stage III NSCLC (e.g., squamous or non-squamous stage III NSCLC)) patient to be treated in accordance with the methods of the present disclosure has or does not have epidermal growth factor receptor (EGFR) sensitizing (activating) mutation. In some embodiments, the treatment naïve cancer (e.g., advanced stage III NSCLC (e.g., squamous or non-squamous stage III NSCLC) with high PD-L1 expression; PD-L1 positive advanced stage III NSCLC (e.g., squamous or non-squamous stage III NSCLC); or PD-L1 negative advanced stage III NSCLC (e.g., squamous or non-squamous stage III NSCLC)) patient to be treated in accordance with the methods of the present disclosure has or does not have anaplastic lymphoma kinase (ALK) translocation. In some embodiments, the treatment naïve cancer (e.g., advanced stage III NSCLC (e.g., squamous or non-squamous stage III NSCLC) with high PD-L1 expression; PD-L1 positive advanced stage III NSCLC (e.g., squamous or non-squamous stage III NSCLC); or PD-L1 negative advanced stage III NSCLC (e.g., squamous or non-squamous stage III NSCLC)) patient to be treated in accordance with the methods of the present disclosure has or does not have ROS1 mutation.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: Packaging of Intravenous Drug Formulation

The formulation of anti-PD-L1/TGFβ Trap is prepared as a lyophilized formulation or a liquid formulation. For preparing the lyophilized formulation, freeze-dried anti-PD-L1/TGFβ Trap is sterilized and stored in single-use glass vials. Several such glass vials are then packaged in a kit for delivering a specific body weight independent dose to a subject diagnosed with a cancer or a tumor. Depending on the dose requirement, the kit contains 12-60 vials. Alternatively, the formulation is prepared and packaged as a liquid formulation and stored as 250 mg/vial to 1000 mg/vial. For example, the formulation is a liquid formulation and stored as 600 mg/vial, or stored as 250 mg/vial. In another example, the anti-PD-L1/TGFβ Trap is formulated as a 10 mg/mL solution and is supplied in USP/Ph Eur type I vials filled to allow an extractable volume of 60 mL (600 mg/60 mL) and closed with rubber stoppers in serum format complying with USP and Ph Eur with an aluminum crimpseal closure.

A subject diagnosed with stage III NSCLC is intravenously administered a formulation containing 500 mg to 2400 mg of anti-PD-L1/TGFβ Trap. For example, the subject is intravenously administered 1200 mg of anti-PD-L1/TGFβ Trap once every two weeks or 1800 mg of anti-PD-L1/TGFβ Trap once every three weeks. The intravenous administration is from a saline bag. The amount of the anti-PD-L1/TGFβ Trap administered to a subject is independent of the subject's body weight.

### Example 2: Effects of Anti-PD-L1/TGFβ Trap in Mitigating cCRT-Induced Fibrosis

In this example experiments performed to evaluate effects of anti-PD-L1/TGFβ Trap administered in combination with radiation or chemotherapy on mitigating pulmonary fibrosis are described.

Cell lines: 4T1 murine breast cancer cells, obtained from the American Type Culture Collection (ATCC), were cultured in RPMI1640 medium supplemented with 10% heat-inactivated fetal bovine serum (FBS) (Life Technologies). All cells were cultured under aseptic conditions and incubated at 37° C. with 5% CO₂. Cells were passaged before in vivo implantation and adherent cells were harvested with TrypLE Express (Gibco) or 0.25% trypsin.

Mice: BALB/c were obtained from Charles River Laboratories. All mice used for experiments were 6- to 12-week-old females. Mice were housed with ad libitum access to food and water in pathogen-free facilities.

Murine Tumor Models: 4T1 cells (approximately 0.5×10⁵) were inoculated intramuscularly (i.m.) in the thigh of BALB/c mice 6 days before treatment initiation. Treatment was initiated 6 days later (day 0), and mice were sacrificed on day 6 (i.e., 12 days after i.m.).

Treatment: For all studies, mice were randomized into treatment groups on the day of treatment initiation (day 0).

Anti-PD-L1/TGF/3 Trap and controls: Anti-PD-L1/TGFβ Trap of the present disclosure is a full human immunoglobulin 1 (IgG1) monoclonal antibody against human PD-L fused to the extracellular domain of human TGF-β receptor II (e.g., including a first polypeptide that includes the amino acid sequence of SEQ ID NO: 3, and a second polypeptide that includes the amino acid sequence of SEQ ID NO: 1). The isotype control is a mutated version of anti-PD-L1, which completely lacks PD-L1 binding. In tumor-bearing mice, anti-PD-L/TGFβ Trap (492 μg) or isotype control (400 μg) were administered with an intravenous injection (i.v.) in 0.2 mL PBS on day 0, 2, and 4. Non-tumor-bearing BALB/c mice were injected i.v. with anti-PD-L1/TGFβ Trap (20 mg/kg), anti-PD-L1 (16.3 mg/kg), Trap control (anti-PD-L1(mut)/TGF-β trap, 20 mg/kg), or isotype control (anti-PD-L1(mut), 16.3 mg/kg).

CCl₄ (carbon tetrachloride)-dependent induction of fibrosis: Mice were weighed and injected with 1:3 CCL₄/Olive oil solution, i.p. at 1 μL/g with a glass Hamilton syringe and 27G×½ needle 2 days a week.

Radiation: To assess the combination of radiation with anti-PD-L1/TGFβ Trap mice were randomized into the following treatment groups: isotype control (133, 400 g)+vehicle control (0.2 mL), radiation (3.6, 7.5, 8 Gy/day), anti-PD-L1/TGFβ Trap (164, 492 g), or anti-PD-L1/TGFβ Trap+radiation. To deliver radiation treatment, a collimator device with lead shielding was used to localize delivery to the tumor-bearing thigh of mice. This region was irradiated by timed exposure to a Cesium-137 gamma irradiator (GammaCell® 40 Exactor, MDS Nordion, Ottawa, ON, Canada). Radiation treatment was given once per day for four days.

### CCl₄-Induced Liver Fibrosis

Histology: Left liver samples were sent to Histotox (Boulder, Colo.) for processing and staining. A 5 m section from the upper, middle, and lower sections of the medial lobe was stained for αSMA (Abcam, cat #ab124964, 1:200) and picrosirius red by standard histology methods. To optimize the slide for morphometric analysis secondary or background staining was omitted so that only positively stained cells or structures were shown. Primary antibodies were labelled using the Agilent Envision+ Rabbit HRP kit (cat #K4011) which includes the secondary HRP labelled antibodies allowing for DAB development.

SMAD/phosphoSMAD analysis: Tissue lysing solution containing 0.02% of HALT protease inhibitor cocktail (Thermo) and 1 mM of EDTA in RIPA buffer (Sigma) was added to frozen liver samples in a proportion of 1:2 weight:volume while thawing. Samples were then homogenized using bead disruption in the Tissuelyser (Qiagen) for 2 minutes/sec at a frequency of 30/sec. After disruption, lysates were centrifuged at 12,000 rpm, 20 minutes, 4° C. The supernatant was aliquoted and filtered through 20 μM mesh filter plates (EMD Millipore). Final lysate was frozen in −80° C. for later analysis or directly measured using SMAD2/3 and phosphoSMAD 2/3 ELISA (Cell Signaling) according to manufacturer's instructions.

Morphometric analysis: Slides were digitally scanned using the Hamamatsu Nanozoomer Scanner and Digital Pathology Software. Saved images were reviewed and reduced to 1.5% zoom using Hamamatsu NDP.view software. Final images were analyzed by threshold analysis of positively stained cells using Image Pro Premier. The same threshold was applied to all the tissues. Images represent the average result.

RNA-seq analysis: RNA was mapped against the Ensembl 75 mouse genome (GRCm38 February 2014), aligned with Bowtie 2 (Langmead & Salzber (2012), Nat. Methods, 9(4):357-359), and quantified with RSEM (Li, B., & Dewey (2011), BMC Bioinformatics, 12:323).

Signature scores were defined as the mean log₂(fold-change) among all genes in each gene signature. These were calculated by adding a pseudocount of 0.5 TPM to all genes and samples, determining the log₂ (TPM), then subtracting the median log₂-TPM for each gene across all samples from the log₂-TPM for each gene. Signature scores for gene sets and expression (log 2 fold-change) of individual genes are shown as boxplots indicating median and 25^{th} and 75^{th} percentiles; whiskers span minimum to maximum.

α-SMA immunohistochemistry: Isolated tumors were fixed in 10% neutral buffered formalin (NBF) for 24 hours at room temperature, dehydrated, and embedded in paraffin wax. Tissues were sectioned at 5 m and transferred to positively charged slides. Prior to staining, sections were deparaffinized and rehydrated. Anti-α-SMA immunohistochemistry was performed using established protocols and the Leica BOND-RX autostainer. Briefly, antigen retrieval was performed using epitope retrieval solution 2 (Leica, cat #AR9640) at 95° C. for 20 minutes. Following blocking, sections were then incubated with HRP-conjugated 5 g/ml anti-α-SMA antibody (clone 1A4, Sigma, cat #SAB420067) for 60 min. Detection was performed using diaminobenzidine substrate (DAB) and sections were counterstained with hematoxylin. After completion of staining, slides were dehydrated and cover-slipped. Stained sections were imaged using the Hamamatsu Nanozoomer microscope.

Digital quantitation of images was performed using Aperio ImageScope Software (Version 12.3.2.8013). For each tumor, multiple regions of interest (ROIs) (8-11 ROIs) were analyzed. Necrotic regions and tumor edges were excluded from analysis. Total positive pixels above background for DAB were determined and divided by the total number of pixels within the ROI to obtain percent positivity for each ROI. Percent positivity scores for each tumor, obtained by averaging ROI values, were plotted using GraphPad Prism.

Statistical analyses: Statistical analyses were performed using GraphPad Prism Software, version 7.0. For pSMAD and picrosirius red analysis, unpaired two-tailed t-tests were used to compare treatments to isotype control. To assess differences in gene signature scores between treatment groups one-way analysis of variance (ANOVA) was performed followed by Tukey's multiple comparison test.

### Anti-PD-L1/TGF/Trap and Trap Control, but not Anti-PD-L1, Decrease Chemotherapy-Induced Fibrosis

CCl₄-induced liver fibrosis in BALB/c mice: To evaluate the in vivo anti-fibrotic effects of anti-PD-L1/TGFβ Trap, liver fibrosis model induced by carbon tetrachloride (CCl₄) chemotherapy treatment was utilized. BALB/c mice were treated with CCl₄ two times a week for six weeks along with three doses of either isotype control, anti-PD-L1, Trap control, or anti-PD-L1/TGFβ Trap.

In this experiment five groups of mice were used: BALB/c mice that were left untreated (Nv) (n=4 mice/group), BALB/c mice that were treated (n=8 mice/group) with CCl₄ (1 L/g, i.p.; 2 days per week for 6 weeks), and BALB/c mice that were treated either isotype control (16.3 mg/kg i.v.; day 0, 2, 4), anti-PD-L1 (16.3 mg/kg, i.v.; day 0, 2, 4), Trap control (20 mg/kg, i.v.; day 0, 2, 4), or anti-PD-L1/TGFβ Trap (20 mg/kg, i.v.; day 0, 2, 4).

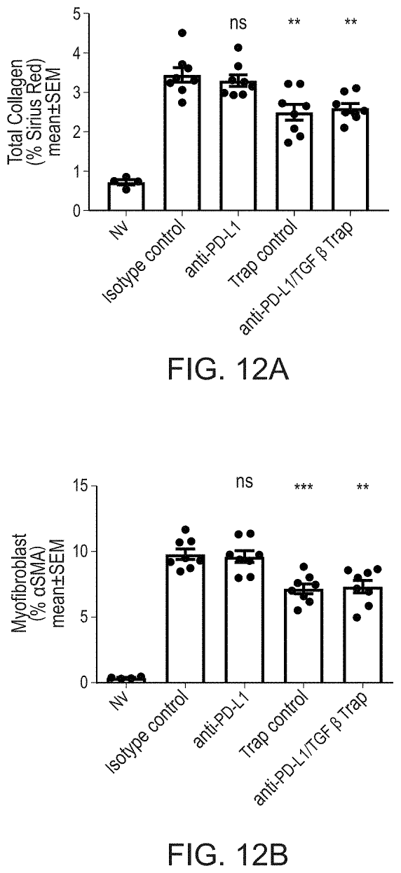

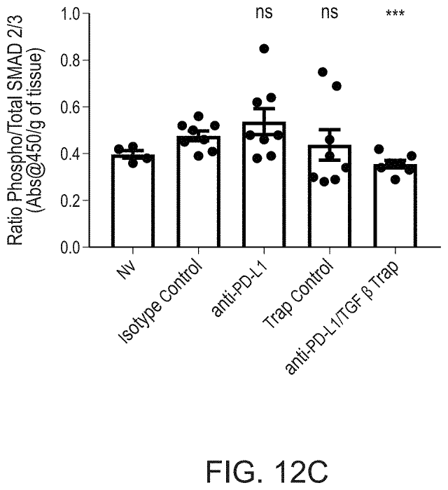

Mice were harvested after 6 weeks and livers were stained for picrosirius red or pSMAD2/3. CCl₄ significantly increased total collagen content, as measured by the percent picrosirius red, in the livers of isotype control mice. While anti-PD-L1 antibody did not affect the collagen content relative to isotype control, both Trap control and anti-PD-L1/TGFβ Trap treatment significantly decreased collagen content (total collagen (percent picrosirius red); p=0.0038 and p=0.0019, respectively) (FIG. 12A). The percent αSMA, a marker of myofibroblasts, in treated liver samples was similarly unaffected by anti-PD-L1 treatment, however, both Trap control and anti-PD-L1/TGFβ Trap treatment significantly decreased the percent αSMA (p=0.0003 and p=0.0013, respectively) (FIG. 12B). The ratio of pSmad2/3 in relation to total Smad2/3 in treated liver samples was also determined, given that phosphorylation of R-Smads, such as pSmad2/3, can be induced by TGF-β isoforms 1-3 (the ratio of phosphorylated SMAD2/3 versus total SMAD2/3 are represented as mean±SEM with each dot representing an individual mouse). Treatments were compared to isotype control using unpaired t-tests. ** p≤0.01 and ***p≤0.001 denote significant differences. Anti-PD-L/TGFβ Trap was the only treatment able to reduce the ratio of pSmad2/3 relative to isotype control treatment (p=0.0006) (FIG. 12C).

Combination Therapy with Anti-PD-L1/TGF/Trap and Radiation Therapy Reduced EMT and Pro-Fibrotic Gene Signature Scores

To examine potential mechanisms of action that induced enhanced antitumor activity of the combination therapy, gene expression in 4T1 tumor tissue via targeted RNA sequencing (RNAseq) was profiled.

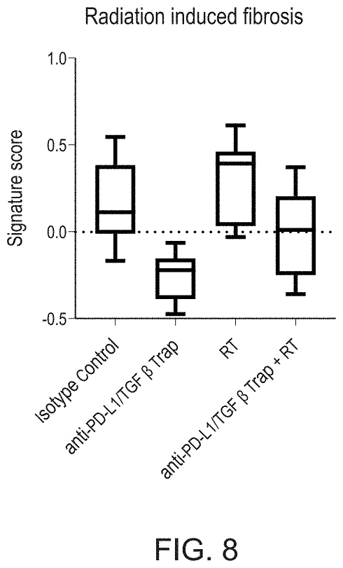

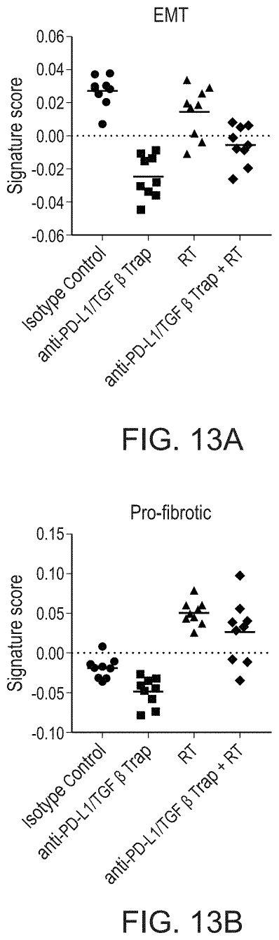

FIGS. 13A, 13B, and FIG. 8 present data from an RNAseq analysis in the 4T1 model. BALB/c mice were inoculated intramuscularly (i.m.) with 0.5×10⁵ 4T1 cells (day −6) and treated (n=10 mice/group) with isotype control (400 g i.v.; day 0, 2, 4)+vehicle control (0.2 mL, orally (per os (p.o.)), twice daily (q.d.), day 0-6), anti-PD-L1/TGFβ Trap (492 g, intravenously (i.v.); day 0, 2, 4), radiation (8 gray (Gy), day 0-3), or anti-PD-L/TGFβ Trap+RT. Mice were sacrificed on day 6 and tumors were collected and processed for RNA extraction. RNAseq was performed with Qiaseq targeted RNA panel and signature scores were defined. Signature scores (defined as the mean log 2 fold-change among all genes in the signature) for EMT and pro-fibrotic genes are presented as scatterplots or box-whisker plots. Whiskers span minimum to maximum.

As part of the RNA sequencing analysis, genes were classified into functional groups and “signature scores” were determined as a measure of gene expression. Anti-PD-L1/TGFβ Trap monotherapy resulted in a reduction in the epithelial-mesenchymal transition (EMT) signature score relative to isotype control (p<0.0001). Although the addition of radiation therapy did not significantly affect the EMT signature (p>0.05), the combination of anti-PD-L1/TGFβ Trap and radiation therapy significantly downregulated the EMT signature score relative to isotype control (p<0.0001) (FIG. 13A).

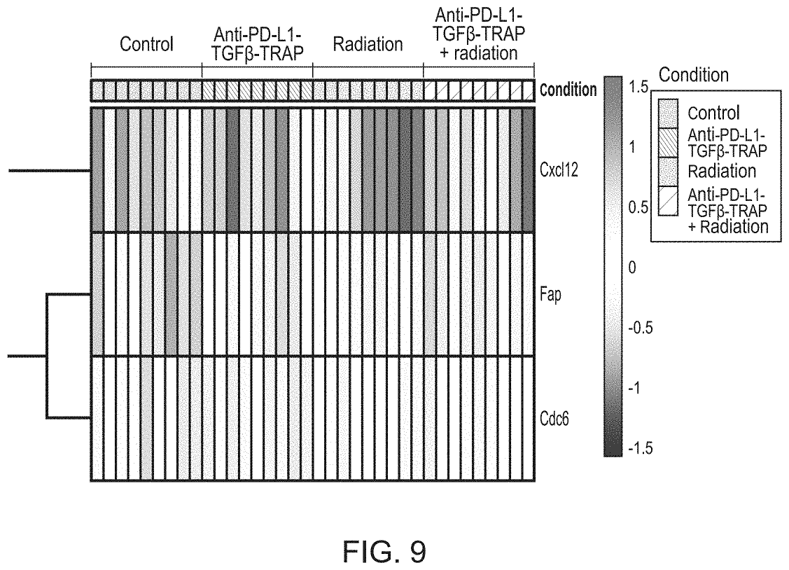

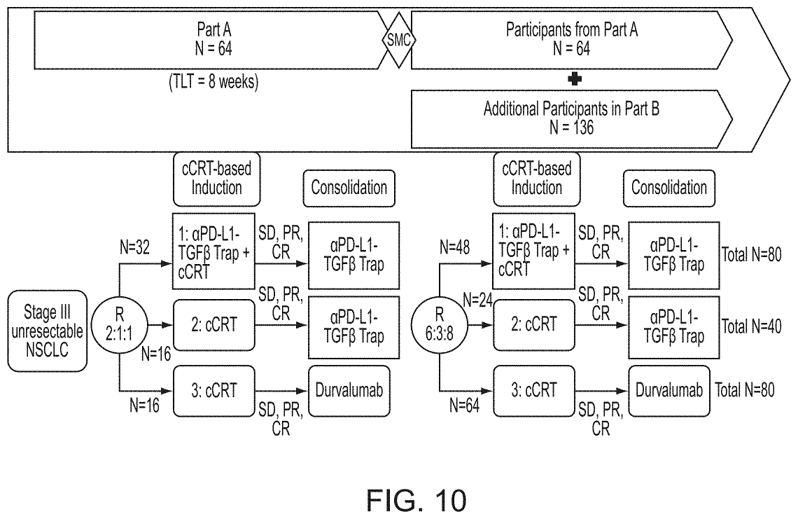

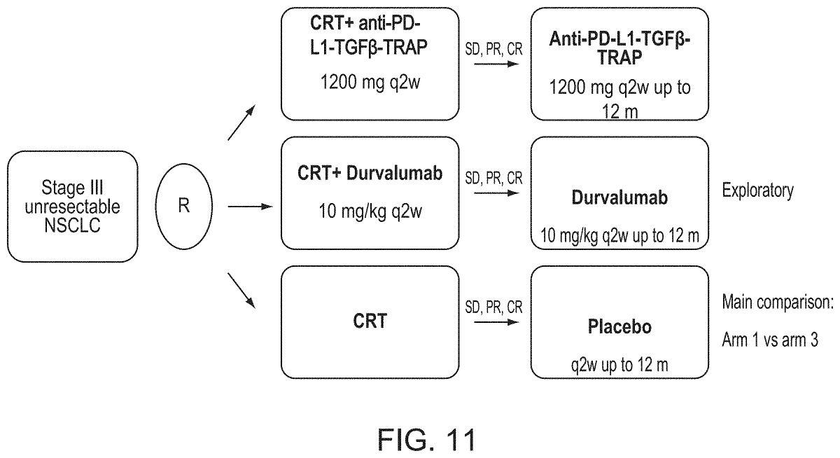

Pro-fibrotic gene signature scores were also decreased by anti-PD-L1/TGFβ Trap monotherapy but were significantly increased by radiation therapy relative to isotype control (p<0.0001). Furthermore, combining radiation with anti-PD-L1/TGFβ Trap reduced pro-fibrotic signature score relative to radiation alone (FIG. 13B). Radiation-induced fibrosis gene signature scores (based on Alsner et al., (2007) Radiotherapy and Oncology, 83(3):261-266) were similarly decreased after anti-PD-L1/TGFβ Trap treatment (p=0.0014). Notably, relative to radiation monotherapy, anti-PD-L1/TGFβ Trap treatment was able to significantly decrease the radiation-induced fibrosis signature when combined with radiation treatment (p=0.0365) (FIG. 8). For the radiation-induced fibrosis gene signature score, expression levels of Cdc6, Cxcl12, and Fap were measured. At level of single genes, Fap was down-regulated 27.1%, adjusted p-value 0.0107 by limma (the adjustment being overall genes measured). Changes in Cdc6 and Cxcl12 were not significant (FIG. 9). Ctgf, a key driver of fibrosis, was also downregulated 34.4% in this comparison, p=0.00350.

To further evaluate the effects of anti-PD-L1/TGFβ Trap and radiation therapy on the EMT and fibrosis, expression of individual genes related to fibroblasts and EMT was quantified.

Expression of smooth muscle a actin (ACTA2) is restricted to smooth muscle cells, pericytes, and myofibroblasts, and is important in myofibroblast function. BALB/c mice were inoculated intramuscularly (i.m.) with 0.5×10⁵ 4T1 cells (day −6) and treated (n=10 mice/group) with isotype control (400 μg i.v.; day 0, 2, 4)+vehicle control (0.2 mL, orally (per os (p.o.)), twice daily (q.d.), day 0-6), anti-PD-L1/TGFβ Trap (492 μg, intravenously (i.v.); day 0, 2, 4), radiation (8 Gy, day 0-3), or anti-PD-L1/TGFβ Trap+RT. Mice were sacrificed on day 6 and tumors were collected and processed for RNA extraction. RNAseq was performed with Qiaseq targeted RNA panel and signature scores were defined. The gene expression (log 2fold change) in each treatment are represented in box-whisker plots for Acta2, Ctgf, and Fap. Whiskers span minimum to maximum.

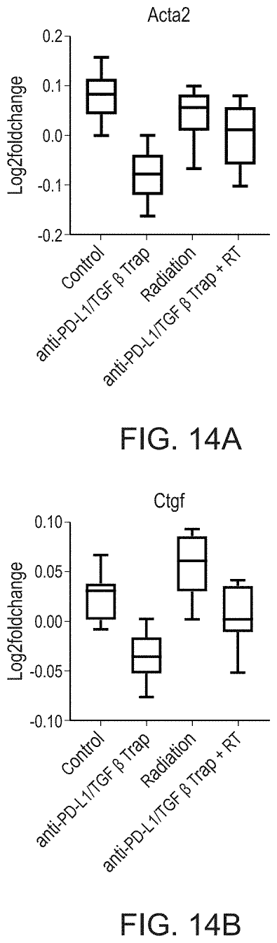

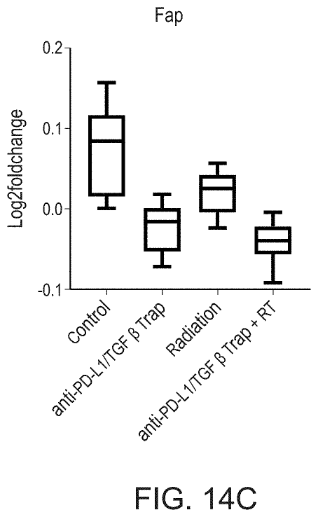

In vitro, ACTA2 expression is increased by TGF-β1 treatment. While radiation treatment alone had no significant effect on ACTA2 expression, anti-PD-L1/TGFβ Trap monotherapy and anti-PD-L1/TGFβ Trap combined with radiation therapy significantly reduced ACTA2 expression in the 4T1 model (p<0.0001 and p=0.0236, respectively) (FIG. 14A).

Connective tissue growth factor (CTGF) is a secreted protein shown to be a central mediator of tissue remodeling and fibrosis. CTGF inhibition has even been shown to reverse the fibrosis process and a monoclonal antibody that targets CTGF, significantly reduced radiation-induced lung fibrosis in mouse models. Anti-PD-L1/TGFβ Trap significantly reduced CTGF expression relative to isotype control (p=0.0019) and, while radiation treatment increased CTGF, as expected, anti-PD-L1/TGFβ Trap combination significantly counteracted the effects of radiation treatment compared to radiation monotherapy (P=0.0024) (FIG. 14B).

Fibroblast activating protein (FAP) is highly expressed by cancer-associated fibroblasts (CAFs) in over 90% of human epithelial cancers, where is can promote immunosuppression by CAFs in the TME via STAT3 signaling. Anti-PD-L1/TGFβ Trap significantly reduced FAP expression relative to isotype control (p<0.0001) and the reduction in FAP seen with radiation therapy was further reduced by the combination of anti-PD-L1/TGFβ Trap with radiation (P=0.0054) (FIG. 14C).

### Anti-PD-L1/TGFβ Trap Treatment Reduces α-SMA Expression in Mouse Tumors

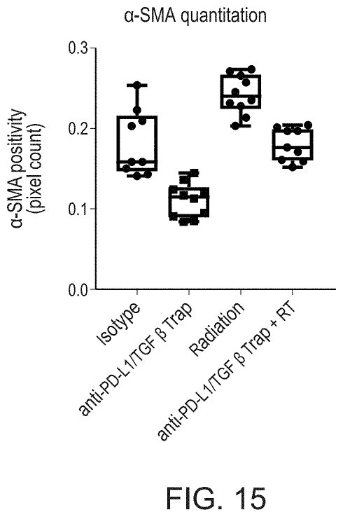

Increased TGF-0 activity induces expression of alpha-smooth muscle actin (α-SMA), a marker of CAFs, which can contribute to drug resistance and are emerging as immunotherapy targets (Calon et al. (2014), Semin. Cancer Biol. 25:15-22; Kakarla et al. (2012), Immunotherapy, 4(11): 1129-1138). To evaluate the effect of anti-PD-L1/TGFβ Trap and radiation therapy on α-SMA expression, α-SMA IHC was performed in the 4T1 tumor sections. BALB/c mice were inoculated intramuscularly (i.m.) with 0.5×10⁵ 4T1 cells (day −6) and treated (n=10 mice/group) with isotype control (400 g i.v.; day 0, 2, 4)+vehicle control (0.2 mL, p.o., twice daily (q.d.), day 0-6), anti-PD-L1/TGFβ Trap (492 g i.v.; day 0, 2, 4), radiation (8 Gy, day 0-3), or anti-PD-L1/TGFβ Trap+radiation. In the box-plots shown in FIG. 15, for quantification, the number of α-SMA+ pixels were determined for multiple regions of interest (ROIs) per tumor and normalized to ROI area; each symbol represents the proportion of positive pixels for a single tumor. P-values were determined by one-way ANOVA. Scale bars, 250 μm.

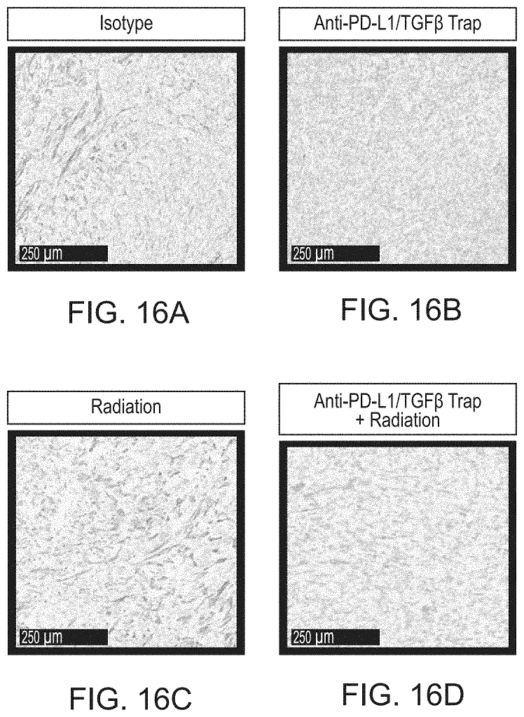

Representative images of anti-α-SMA IHC are shown (FIGS. 16A-16D). Relative to isotype control (FIG. 16A), anti-PD-L1/TGFβ Trap treatment significantly reduced α-SMA expression (p<0.0001) (FIG. 16B), while radiation therapy significantly increased α-SMA expression (p=0.0002) (FIG. 16C). The combination of anti-PD-L1/TGFβ Trap with radiation therapy significantly reduced α-SMA expression relative to radiation monotherapy (P=0.0001) (FIG. 16D), suggesting that anti-PD-L1/TGFβ Trap can reduce radiation-induced CAF activity.

### Example 3: Anti-PD-L1/TGFβ Trap Administration with Concomitant Chemotherapy and Radiotherapy (cCRT) of a Treatment Naïve, Stage III NSCLC Patient Cohort—Study Design 1

Treatment-naïve patients with stage III non-small cell lung cancer (NSCLC) are treated with anti-PD-L1/TGFβ Trap in combination with cCRT followed by anti-PD-L1/TGFβ Trap for consolidation (Arm 1), and compared to patients enrolled in cCRT followed by consolidation treatment with anti-PD-L1/TGFβ Trap (Arm 2) and patients treated with cCRT followed by durvalumab (Arm 3). In one exemplary embodiment, cCRT is administered as either cisplatin/etoposide, cisplatin/pemetrexed, or carboplatin/paclitaxel concurrently with 60-66 Gy (e.g., 60 Gy) total dose of radiation delivered by intensity-modulated radiation therapy. Chemotherapy regimen is the stratification factor.

In one exemplary embodiment, anti-PD-L1/TGFβ Trap is administered as a BW-independent dose of 1200 mg to cancer patients with stage III non-small cell lung cancer (NSCLC) once every two weeks. The administration is performed intravenously for about an hour (−10 minutes/+20 minutes, e.g., 50 minutes to 80 minutes). In one exemplary embodiment, anti-PD-L1/TGFβ Trap is administered as a BW-independent dose of 1800 mg to cancer patients with stage III non-small cell lung cancer (NSCLC) once every three weeks. The administration is performed intravenously for about an hour (−10 minutes/+20 minutes, e.g., 50 minutes to 80 minutes). In one exemplary embodiment, anti-PD-L1/TGFβ Trap is administered as BW-independent dose of 2400 mg to cancer patients with stage III non-small cell lung cancer (NSCLC) once every three weeks. The administration is performed intravenously for about an hour (−10 minutes/+20 minutes, e.g., 50 minutes to 80 minutes). In one or more exemplary embodiments, in order to mitigate potential infusion-related reactions, premedication with an antihistamine and with paracetamol (acetaminophen) (for example, 25-50 mg diphenhydramine and 500-650 mg paracetamol [acetaminophen] IV or oral equivalent) approximately 30 to 60 minutes prior to each anti-PD-L1/TGFβ Trap dose is administered for the first 2 infusions. If Grade ≥2 infusion reactions are observed during the first two infusions, premedication is not stopped. Steroids as premedication are not permitted.

The following describes the inclusion criteria for patients used in this example. Patients:
are ≥18 years, inclusive at the time of informed consent
have histologically or cytologically confirmed diagnosis of locally advanced, unresectable (Stage III) NSCLC
at least 3 weeks since prior thoracotomy (if performed)
have not received prior systemic therapy treatment, or any antibody or drug targeting T-cell coregulatory proteins (immune checkpoints) such as anti-PDL1, or anti-CTLA-4 antibody, since diagnosis of stage III NSCLC
have a life expectancy of at least 12 weeks (based on physician's assessment of the prognosis of the patient after diagnosis)
have available tumor material (<6 months old) adequate for biomarker analysis
have Eastern Cooperative Oncology Group Performance Status (ECOG PS) of 0 to 1
have adequate pulmonary function defined as a forced expiratory volume in 1 second (FEV₁)≥1.2 liters or ≥50% of predicted normal volume measured within 3 weeks prior to randomization
have adequate hematological function defined by absolute neutrophil count (ANC)≥1.5×10⁹/L, platelet count ≥100×10⁹/L, and Hgb≥9 g/dL
have adequate hepatic function defined by a total bilirubin level ≤1.5× upper limit of normal (ULN), an aspartate aminotransferase (AST) level ≤3.0×ULN, an alanine aminotransferase (ALT) level ≤3.0×ULN and alkaline phosphatase ≤2.5 ULN.
have adequate renal function defined by creatine ≤1.5×ULN or calculated creatinine clearance (CrCl) ≥50 mL/min for participant with Cr>1.5×ULN (GFR can also be used)
have adequate coagulation function defined as international normalized ratio (INR) or prothrombin time (PT)≤1.5×ULN unless the participant is receiving anticoagulant therapy, and activated partial thromboplastin time (aPTT)≤1.5×ULN unless the participant is receiving anticoagulant therapy.

### Example 4: Anti-PD-L1/TGFβ Trap Administration with Concomitant Chemotherapy and Radiotherapy (cCRT) of a Treatment Naïve, Stage III NSCLC Patient Cohort—Study Design 2

Treatment-naïve patients with stage III non-small cell lung cancer (NSCLC) are treated with anti-PD-L1/TGFβ Trap in combination with cCRT followed by anti-PD-L1/TGFβ Trap for consolidation (Arm 1), and compared to patients treated with 10 mg/kg biweekly durvalumab in combination with cCRT followed by consolidation treatment with 10 mg/kg biweekly durvalumab (Arm 2) and also to patients treated with cCRT alone followed by placebo (Arm 3). In one exemplary embodiment, cCRT is administered as either cisplatin/etoposide, cisplatin/pemetrexed, or carboplatin/paclitaxel concurrently with 60-66 Gy (e.g., 60 Gy) total dose of radiation delivered by intensity-modulated radiation therapy. Chemotherapy regimen is the stratification factor.

In one exemplary embodiment, anti-PD-L1/TGFβ Trap is administered as a BW-independent dose of 1200 mg to cancer patients with stage III non-small cell lung cancer (NSCLC) once every two weeks. The administration is performed intravenously for about an hour (−10 minutes/+20 minutes, e.g., 50 minutes to 80 minutes). In one exemplary embodiment, anti-PD-L1/TGFβ Trap is administered as a BW-independent dose of 1800 mg to cancer patients with stage III non-small cell lung cancer (NSCLC) once every three weeks. The administration is performed intravenously for about an hour (−10 minutes/+20 minutes, e.g., 50 minutes to 80 minutes). In one exemplary embodiment, anti-PD-L1/TGFβ Trap is administered as BW-independent dose of 2400 mg to cancer patients with stage III non-small cell lung cancer (NSCLC) once every three weeks. The administration is performed intravenously for about an hour (−10 minutes/+20 minutes, e.g., 50 minutes to 80 minutes). In one or more exemplary embodiments, in order to mitigate potential infusion-related reactions, premedication with an antihistamine and with paracetamol (acetaminophen) (for example, 25-50 mg diphenhydramine and 500-650 mg paracetamol [acetaminophen] IV or oral equivalent) approximately 30 to 60 minutes prior to each anti-PD-L1/TGFβ Trap dose is administered for the first 2 infusions. If Grade 2 infusion reactions are observed during the first two infusions, premedication is not stopped. Steroids as premedication are not permitted.

Inclusion criteria for Example 4 are similar as for Example 2, but could be adjusted according to the judgment of the investigator.

### Example 5: Therapeutic Efficacy in Treatment of Stage III NSCLC Patients with Anti-PD-L1/TGFβ Trap

Progression-free survival (PFS) according to RECIST 1.1 is measured as a primary endpoint in participants treated with anti-PD-L1/TGFβ Trap in combination with cCRT as described in Examples 3 and 4. The difference in efficacy between the arms of each example is investigated.

In one exemplary embodiment, cisplatin is administered at a dose of 50 mg/m² intravenously over 60 minutes or according to local standards on Days 1, 8, 29, 36 during cCRT-based induction. Etoposide is administered at a dose of 50 mg/m² intravenously over a minimum of 30 minutes up to 60 minutes daily on days 1-5, 29-33 during cCRT-based induction.

Standard premedication consisting of an H2-blocker, antiemetics, dexamethasone (oral or intravenous) are administered according to local guidelines. Adequate hydration pre- and post-treatment in participants receiving cisplatin/etoposide is ensured according to the local practice.

In one exemplary embodiment, paclitaxel is administered intravenously at a dose of 45 mg/m² over 60 minutes or according to local prescribing information on day 1 of every week during cCRT-based induction. Standard premedication consisting of diphenhydramine 25-50 mg, an H2-blocker, and dexamethasone (oral or IV is acceptable) according to local standards is given at least 30 minutes prior to paclitaxel.

For participants who are treated with carboplatin/paclitaxel regimen, and are not able to receive anti-PD-L1/TGFβ Trap or durvalumab as consolidation, 2 additional cycles of carboplatin/paclitaxel (carboplatin AUC 6, paclitaxel 200 mg/m², Q3W) are given as consolidation treatment per investigator decision.

Carboplatin is administered intravenously based on AUC 2 over 30 minutes or according to local standards on Day 1 of every week during the cCRT-based induction. Carboplatin will be given with standard antiemetics after the paclitaxel is administered.

The therapeutic efficacy can also be measured with three additional outcome determinants. The therapeutic efficacy can be measured as Objective Response Rate (ORR), which according to the U.S. Food and Drug Administration is the “proportion of patients with a tumor size reduction of predefined amount and for a minimum period of time.” See FDA 2007. Complete response, according to the National Cancer Institute (NCI, USA) is the “disappearance of all signs of cancer in response to treatment.” ORR is the preferred measure of therapeutic efficacy over CR. See Kogan & Haren (2008), Biotech. Healthcare, 5(1):22-35. Another measure of therapeutic efficacy is overall survival (OS), which is the time from randomization to planned assessment, for example, at 57 months. The therapeutic efficacy can also be measured as duration of response (assessed from CR or partial response (PR) until progression of disease (PD), death, or last tumor assessment), which is the time from randomization to planned assessment, for example, at 57 months.

Contrast-enhanced computed tomography (CT) of chest/abdomen and pelvis covering the area from the superior extent of the thoracic inlet to the symphysis pubis is the first choice of imaging modality to assess treatment efficacy. The tumor assessment prior to consolidation is performed close as possible before the start of the consolidation treatment, and within 14 days after the end of CRT-based induction. For patients who are recovering from toxicities associated with cCRT, the start of consolidation is delayed by up to 42 days from the end of the cCRT. Participants are evaluated every 6 weeks with radiographic imaging to assess response to treatment within the 15 months of the participant's first dose, then every 12 weeks thereafter.

Additional endpoints are investigated to further establish therapeutic efficacy. For example, changes in tumor size are evaluated by tumor volumetric analysis compared to baseline, and changes in tumor metabolic volume are measured with PET scan. Changes from baseline in lung fibrosis are measured with high resolution CT scan and pulmonary function tests. Potential predictive biomarkers of clinical response are evaluated by examining mutation types and numbers (Tumor Mutational Burden (TMB)) in plasma or in tumor tissue, and investigating the correlation between TMB and clinical outcome.

It is contemplated that treatment with anti-PD-L1/TGFβ Trap results in initial clinical activity in treatment naïve, stage III NSCLC patients. Treated patients exhibit disease response (e.g., partial response, complete response, stable disease) and/or improved survival (e.g., progression-free survival and/or overall survival). It is contemplated that treatment with anti-PD-L1/TGFβ Trap with concomitant cCRT followed by anti-PD-L1/TGFβ Trap consolidation treatment results in superior survival of treatment naïve, stage III NSCLC patients compared to cCRT alone, or patients treated with cCRT followed by placebo.

In summary, anti-PD-L1/TGFβ Trap with concomitant cCRT is found to be an innovative first-in-class bifunctional fusion protein designed to simultaneously target 2 immune suppressive pathways: PD-L1 and TGF-β, and, thereby treat stage III NSCLC, while minimizing the development of fibrosis associated with concomitant radiotherapy, and increasing the time-to-onset of metastasis and/or time to distant metastasis of the stage III NSCLC in the subject.

### Example 6: Anti-PD-L1/TGFβ Trap Administration with Concomitant Chemotherapy and Radiotherapy (cCRT) of an Advanced Unresectable Stage III NSCLC Patient Cohort [Total 350]—Study Design 3

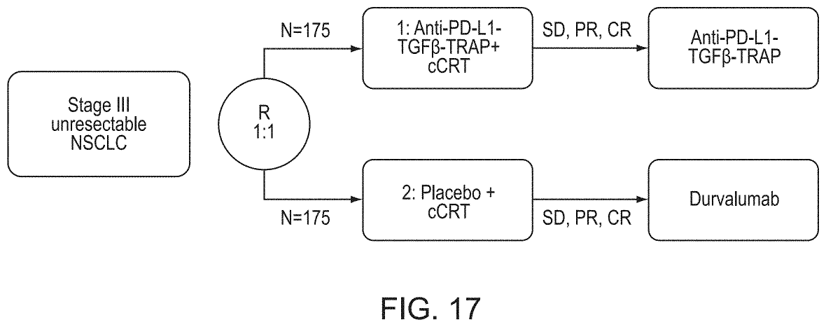

Patients with advanced unresectable stage III non-small cell lung cancer (NSCLC) are treated with anti-PD-L1/TGFβ Trap in combination with cCRT (e.g., platinum-based chemoradiation) followed by anti-PD-L1/TGFβ Trap for consolidation (Arm 1), and compared to patients treated with cCRT along with placebo matched to anti-PD-L1/TGFβ Trap followed by durvalumab (Arm 2). A schematic diagram of the therapeutic regimen is described in FIG. 17. In one exemplary embodiment, cCRT is administered as either cisplatin/etoposide, cisplatin/pemetrexed, or carboplatin/paclitaxel concurrently with 60-66 Gy (e.g., 60 Gy) total dose of radiation delivered by intensity-modulated radiation therapy. Chemotherapy regimen and/or PD-L1 expression are the stratification factors in the study.

In one exemplary embodiment, anti-PD-L1/TGFβ Trap is administered as a BW-independent dose of 1200 mg to cancer patients with stage III non-small cell lung cancer (NSCLC) once every two weeks. The administration is performed intravenously for about an hour (−10 minutes/+20 minutes, e.g., 50 minutes to 80 minutes). In one exemplary embodiment, anti-PD-L1/TGFβ Trap is administered as a BW-independent dose of 1800 mg to cancer patients with stage III non-small cell lung cancer (NSCLC) once every three weeks. The administration is performed intravenously for about an hour (−10 minutes/+20 minutes, e.g., 50 minutes to 80 minutes). In one exemplary embodiment, anti-PD-L1/TGFβ Trap is administered as BW-independent dose of 2400 mg to cancer patients with stage III non-small cell lung cancer (NSCLC) once every three weeks. The administration is performed intravenously for about an hour (−10 minutes/+20 minutes, e.g., 50 minutes to 80 minutes). In one or more exemplary embodiments, in order to mitigate potential infusion-related reactions, premedication with an antihistamine and with paracetamol (acetaminophen) (for example, 25-50 mg diphenhydramine and 500-650 mg paracetamol [acetaminophen] IV or oral equivalent) approximately 30 to 60 minutes prior to each anti-PD-L1/TGFβ Trap dose is administered for the first 2 infusions. If Grade ≥2 infusion reactions are observed during the first two infusions, premedication is not stopped. Steroids as premedication are not permitted.

In one exemplary embodiment, patients with advanced unresectable stage III NSCLC are intravenously infused with 1200 mg of anti-PD-L1/TGFβ Trap over 1 hour every two weeks until unacceptable toxicity, confirmed disease progression, during cCRT and up to 1 year after cCRT. In one exemplary embodiment, 4 doses (e.g., 1200 mg each) of anti-PD-L1/TGFβ Trap are administered during induction phase concomitant with cCRT. In one or more exemplary embodiments, 26 doses (e.g., 1200 mg each) of anti-PD-L1/TGFβ Trap are administered during consolidation phase.

In one exemplary embodiment, etoposide is administered at a dose of 50 mg/m² or according to local standards intravenously over a minimum of 30 minutes up to 60 minutes daily on days 1-5 and 29-33 during cCRT. In one exemplary embodiment, pemetrexed is administered at a dose of 500 mg/m² or according to local standards intravenously over 10 minutes or according to local standards on days 1, 22, and 43 during cCRT. In one exemplary embodiment, carboplatin is administered intravenously based on area under curve (AUC) 2 over 30 minutes on days 1, 8, 15, 22, 29, 36, and 43 during cCRT. In one exemplary embodiment, paclitaxel is administered intravenously at a dose of 45 mg/m² or according to local standards over 60 minutes on days 1, 8, 15, 22, 29, 36, and 43 during cCRT. Standard premedication consisting of diphenhydramine 25-50 mg, an H2-blocker, and dexamethasone (oral or IV is acceptable) according to local standards is given at least 30 minutes prior to paclitaxel.

In one exemplary embodiment, cisplatin is administered at a dose of 50 mg/m² intravenously over 60 minutes or according to local standards on days 1, 8, 29, 36 during cCRT-based induction. Etoposide is administered at a dose of 50 mg/m² intravenously over a minimum of 30 minutes up to 60 minutes daily on days 1-5, 29-33 during cCRT-based induction.

In one exemplary embodiment, cisplatin is administered at a dose of 75 mg/m² intravenously over 60 minutes or according to local standards on Days 1, 22, 43 during cCRT-based induction. Pemetrexed is administered at a dose of 500 mg/m² or according to local standards intravenously over 10 minutes or according to local standards on Days 1, 22, and 43 during cCRT.

In the Arm 2, patients with advanced unresectable stage III NSCLC are intravenously infused with a placebo matched to anti-PD-L1/TGFβ Trap over 1 hour every 2 weeks until acceptable toxicity, confirmed disease progression during cCRT. Durvalumab is administered biweekly at 10 mg/kg over 1 hour until acceptable toxicity, confirmed disease progression during cCRT and up to 1 year after cCRT. In one or more exemplary embodiments, 26 doses (e.g., 10 mg/kg each) of durvalumab are administered during consolidation phase.

In one exemplary embodiment, in order to mitigate potential infusion-related reactions, premedication with an antihistamine and with paracetamol (acetaminophen) (for example, 25-50 mg diphenhydramine and 500-650 mg paracetamol [acetaminophen] IV or oral equivalent) approximately 30 to 60 minutes prior to each anti-PD-L1/TGFβ Trap dose is administered for the first 2 infusions. If Grade ≥2 infusion reactions are observed during the first two infusions, premedication is not stopped. Steroids as premedication are not permitted.

In one exemplary embodiment, standard premedication consisting of an H2-blocker, antiemetics, dexamethasone (oral or intravenous) are administered according to local guidelines. Adequate hydration pre- and post-treatment in participants receiving cisplatin/etoposide is ensured according to the local practice.

The following describes the inclusion criteria for patients used in this example. Patients:
are ≥18 years, inclusive at the time of informed consent
have histologically documented NSCLC who present with Stage III locally advanced, unresectable disease (International Association for the Study of Lung Cancer Staging Manual in Thoracic Oncology)
patients with tumor harboring an Epidermal growth factor receptor (EGFR) sensitizing (activating) mutation, Anaplastic lymphoma kinase (ALK) translocation, ROS-1 rearrangement are eligible
have adequate pulmonary function defined as a forced expiratory volume in 1 second (FEV1) greater than equals to (>=) 1.2 titers or >=50% of predicted normal volume measured within 3 weeks prior to randomization
have adequate hematological function defined by absolute neutrophil count (ANC)≥1.5×10⁹/L, platelet count ≥100×10⁹/L, and hemoglobin ≥9 g/dL
have adequate hepatic function defined by a total bilirubin level ≤1.5× upper limit of normal (ULN), an aspartate aminotransferase (AST) level ≤3.0×ULN, an alanine aminotransferase (ALT) level ≤3.0×ULN and alkaline phosphatase ≤2.5 ULN
have adequate renal function as defined by creatine ≤1.5×ULN or calculated creatinine clearance (CrCl)≤50 mL/min for participant with Cr>1.5×ULN (GFR can also be used)
use contraceptives (males and females) consistent with local regulations on contraception methods
have Eastern Cooperative Oncology Group Performance Status (ECOG PS) of 0 to 1

Patients may be excluded from the study because of any prior systemic cytotoxic chemotherapy for their NSCLC or any antibody or drug targeting T-cell coregulatory proteins.

### Example 7: Therapeutic Efficacy in Treatment of Advanced Unresectable Stage III NSCLC Patients as Described in Example 6

Progression-free survival (PFS) according to RECIST 1.1 is measured as a primary endpoint in participants treated with anti-PD-L1/TGFβ Trap in combination with cCRT followed by anti-PD-L1/TGFβ Trap, as described in Example 6. The difference in efficacy between the arms of each example is investigated.

The therapeutic efficacy can also be measured with additional outcome determinants. A measure of therapeutic efficacy is overall survival (OS), which is the time from randomization to planned assessment, for example, at 59 months. Best Overall Response (BOR), which is the best response recorded from the start of the study treatment until the disease progression/recurrence can also be investigated to further establish therapeutic efficacy. Additional measure of therapeutic efficacy is through evaluation of PD-L1 expression at baseline. Another secondary endpoint is safety. Additional endpoints are investigated to further establish therapeutic efficacy. For example, changes in tumor size are evaluated by tumor volumetric analysis compared to baseline, and changes in tumor metabolic volume are measured with PET scan. Changes from baseline in lung fibrosis are measured with high resolution CT scan and pulmonary function tests.

Contrast-enhanced computed tomography (CT) of chest/abdomen and pelvis covering the area from the superior extent of the thoracic inlet to the symphysis pubis is the first choice of imaging modality to assess treatment efficacy. Participants are evaluated every 8 weeks with radiographic imaging to assess response to study intervention for up to 24 months of the participant's first dose unless progression or withdrawal from the study whichever occurs first. Subsequent scans are done every 8-12 weeks up to progression, start of new treatment or death.

Potential predictive biomarkers of clinical response may be evaluated by examining mutation types and numbers (Tumor Mutational Burden (TMB)) in plasma or in tumor tissue, and investigating the correlation between TMB and clinical outcome.

Additional exploratory endpoints are investigated to further establish therapeutic efficacy. For example, changes in circulating tumor DNA (ctDNA) levels, immune-related Best Overall Response (irBOR) and irunune-related Progression-Free Survival (irPFS) according to immune-related Response Evaluation Criteria in Solid Tumors (irRECIST).

It is contemplated that treatment with anti-PD-L1/TGFβ Trap results in initial clinical activity in treatment of advanced unresectable stage III NSCLC patients. Treated patients exhibit disease response (e.g., partial response, complete response, stable disease) and/or improved survival (e.g., progression-free survival and/or overall survival). It is contemplated that treatment with anti-PD-L1/TGFβ Trap with concomitant cCRT followed by anti-PD-L1/TGFβ Trap consolidation treatment results in superior survival of advanced unresectable stage III NSCLC patients compared to patients treated with cCRT along with placebo matched to anti-PD-L1/TGFβ Trap followed by durvalumab.

In one exemplary embodiment, the PD-L1 expression is determined by an FDA-approved test (e.g., (Tumor Proportion Score (TPS) or the VENTANA PD-L1 (SP263) assay). In one exemplary embodiment, the anti-PD-L1 antibody is used to determine the PD-L1 protein expression in a formalin-fixed, paraffin-embedded tissue. In one exemplary embodiment, patients are enrolled irrespective of PD-L1 expression and stratified retrospectively for PD-L1 expression with SP263 assay. In one exemplary embodiment, PD-L1 data (retrospective and prospective) is considered in the primary efficacy analysis (stratified log-rank test, PD-L-stratified Cox-model, PD-L1 adjusted Cox-model as sensitivity analysis for the estimation of the treatment effect regarding PFS and OS).

In one exemplary embodiment, the chemotherapy regimen (e.g., cisplatin/pemetrexed) is used as a stratification factor in the study. In one exemplary embodiment, patients diagnosed with stage III NSCLC (e.g., squamous or non-squamous) are treated by intravenously administering cisplatin/etoposide or carboplatin/paclitaxel in combination with anti-PD-L1/TGFβ Trap followed by treatment with anti-PD-L1/TGFβ Trap. In one exemplary embodiment, patients diagnosed with stage III NSCLC, with non-squamous histology, are treated by intravenously administering cisplatin/pemetrexed in combination with anti-PD-L1/TGFβ Trap followed by treatment with anti-PD-L1/TGFβ Trap.

In summary, anti-PD-L1/TGFβ Trap with concomitant cCRT is found to be an innovative first-in-class bifunctional fusion protein designed to simultaneously target 2 immune suppressive pathways: PD-L1 and TGF-β, and, thereby treat stage III NSCLC, while minimizing the development of fibrosis associated with concomitant radiotherapy, and increasing the time-to-onset of metastasis and/or time to distant metastasis of the stage III NSCLC in the subject.

SEQUENCES

SEQ ID NO: 1
Peptide sequence of the secreted anti-PD-L1 lambda light chain
QSALTQPASVSGSPGQSITISCTGTSSDVGGYNYVSWYQQHPGKAPKLMIYDVSNRPSG
VSNRFSGSKSGNTASLTISGLQAEDEADYYCSSYTSSSTRVFGTGTKVTVLGQPKANPTV
TLFPPSSEELQANKATLVCLISDFYPGAVTVAWKADGSPVKAGVETTKPSKQSNNKYAA
SSYLSLTPEQWKSHRSYSCQVTHEGSTVEKTVAPTECS
SEQ ID NO: 2
Peptide sequence of the secreted H chain of anti-PDL1
EVQLLESGGGLVQPGGSLRLSCAASGFTFSSYIMMWVRQAPGKGLEWVSSIYPSGGITF
YADTVKGRFTISRDNSKNTLYLQMNSLRAEDTAVYYCARIKLGTVTTVDYWGQGTLVT
VSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKRVEPKSCDKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK
SEQ ID NO: 3
Peptide sequence of the secreted H chain of anti-PDL1/TGFβ Trap
EVQLLESGGGLVQPGGSLRLSCAASGFTFSSYIMMWVRQAPGKGLEWVSSIYPSGGITF
YADTVKGRFTISRDNSKNTLYLQMNSLRAEDTAVYYCARIKLGTVTTVDYWGQGTLVT
VSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKRVEPKSCDKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGAGGGGSGGGGSGGGGSGGGGS
GIPPHVQKSVNNDMIVTDNNGAVKFPQLCKFCDVRFSTCDNQKSCMSNCSITSICEKPQE
VCVAVWRKNDENITLETVCHDPKLPYHDFILEDAASPKCIMKEKKKPGETFFMCSCSSD
ECNDNIIFSEEYNTSNPD
SEQ ID NO: 4
DNA sequence from the translation initiation codon to the translation stop codon of the
anti-PD-L1 lambda light chain (the leader sequence preceding the VL is the signal peptide
from urokinase plasminogen activator)
atgagggccctgctggctagactgctgctgtgcgtgctggtcgtgtccgacagcaagggcCAGTCCGCCCTGACCCAGC
CTGCCTCCGTGTCTGGCTCCCCTGGCCAGTCCATCACCATCAGCTGCACCGGCACCT
CCAGCGACGTGGGCGGCTACAACTACGTGTCCTGGTATCAGCAGCACCCCGGCAAG
GCCCCCAAGCTGATGATCTACGACGTGTCCAACCGGCCCTCCGGCGTGTCCAACAG
ATTCTCCGGCTCCAAGTCCGGCAACACCGCCTCCCTGACCATCAGCGGACTGCAGGC
AGAGGACGAGGCCGACTACTACTGCTCCTCCTACACCTCCTCCAGCACCAGAGTGTT
CGGCACCGGCACAAAAGTGACCGTGCTGggccagcccaaggccaacccaaccgtgacactgttccccccatc
ctccgaggaactgcaggccaacaaggccaccctggtctgcctgatctcagatttctatccaggcgccgtgaccgtggcctggaaggctgat
ggctccccagtgaaggccggcgtggaaaccaccaagccctccaagcagtccaacaacaaatacgccgcctcctcctacctgtccctgac
ccccgagcagtggaagtcccaccggtcctacagctgccaggtcacacacgagggctccaccgtggaaaagaccgtcgcccccaccgag
tgctcaTGA
SEQ ID NO: 5
DNA sequence from the translation initiation codon to the translation stop codon (mVK SP
leader: small underlined; VH: capitals; IgG1m3 with K to A mutation: small letters;
(G4S)x4-G (SEQ ID NO: 11) linker: bold capital letters; TGFβRII: bold underlined small
letters; two stop codons: bold underlined capital letters)
atggaaacagacaccctgctgctgtgggtgctgctgctgtgggtgcccggctccacaggcGAGGTGCAGCTGCTGGAAT
CCGGCGGAGGACTGGTGCAGCCTGGCGGCTCCCTGAGACTGTCTTGCGCCGCCTCCG
GCTTCACCTTCTCCAGCTACATCATGATGTGGGTGCGACAGGCCCCTGGCAAGGGCC
TGGAATGGGTGTCCTCCATCTACCCCTCCGGCGGCATCACCTTCTACGCCGACACCG
TGAAGGGCCGGTTCACCATCTCCCGGGACAACTCCAAGAACACCCTGTACCTGCAG
ATGAACTCCCTGCGGGCCGAGGACACCGCCGTGTACTACTGCGCCCGGATCAAGCT
GGGCACCGTGACCACCGTGGACTACTGGGGCCAGGGCACCCTGGTGACAGTGTCCT
CCgctagcaccaagggcccatcggtcttccccctggcaccctcctccaagagcacctctgggggcacagcggccctgggctgcctggt
caaggactacttccccgaaccggtgacggtgtcgtggaactcaggcgccctgaccagcggcgtgcacaccttcccggctgtcctacagtc
ctcaggactctactccctcagcagcgtggtgaccgtgccctccagcagcttgggcacccagacctacatctgcaacgtgaatcacaagccc
agcaacaccaaggtggacaagagagttgagcccaaatcagtgacaaaactcacacatgcccaccgtgcccagcacctgaactcctggg
gggaccgtcagtatcctatccccccaaaacccaaggacaccctcatgatctcccggacccctgaggtcacatgcgtggtggtggacgtg
agccacgaagaccctgaggtcaagttcaactggtacgtggacggcgtggaggtgcataatgccaagacaaagccgcgggaggagcagt
acaacagcacgtaccgtgtggtcagcgtcctcaccgtcctgcaccaggactggctgaatggcaaggagtacaagtgcaaggtctccaaca
aagccctcccagcccccatcgagaaaaccatctccaaagccaaagggcagccccgagaaccacaggtgtacaccctgcccccatcccg
ggaggagatgaccaagaaccaggtcagcctgacctgcctggtcaaaggcttctatcccagcgacatcgccgtggagtgggagagcaatg
ggcagccggagaacaactacaagaccacgcctcccgtgctggactccgacggetccacttcctctatagcaagcteaccgtggacaaga
gcaggtggcagcaggggaacgtcttctcatgctccgtgatgcatgaggctctgcacaaccactacacgcagaagagcctctccctgtcccc
gggtgaGGCGGCGGAGGAAGCGGAGGAGGTGGCAGCGGTGGCGGTGGCTCCGG
CGGAGGTGGCTCCGGAatccctccccacgtgcagaagtccgtgaacaacgacatgatcgtgaccgacaacaacg
gcgccgtgaagttccctcagctgtgcaagttctgcgacgtgaggttcagcacctgcgacaaccagaagtcctgcatgagcaactgc
agcatcacaagcatctgcgagaagccccaggaggtgtgtgtggccgtgtggaggaagaacgacgaaaacatcaccctcgagacc
gtgtgccatgaccccaagctgccctaccacgacttcatcctggaagacgccgcctcccccaagtgcatcatgaaggagaagaaga
agcccggcgagaccttcttcatgtgcagctgcagcagcgacgagtgcaatgacaacatcatctttagcgaggagtacaacaccag
caaccccgacTGATAA
SEQ ID NO: 6
Polypeptide sequence of the secreted lambda light chain of anti-PD-L1 (mut)/TGFβ Trap,
with mutations A31G ,D52E, R99Y
QSALTQPASVSGSPGQSITISCTGTSSDVGGYNYVSWYQQHPGKAPKLMIYEVSNRPSG
VSNRFSGSKSGNTASLTISGLQAEDEADYYCSSYTSSSTYVFGTGTKVTVLGQPKANPTV
TLFPPSSEELQANKATLVCLISDFYPGAVTVAWKADGSPVKAGVETTKPSKQSNNKYAA
SSYLSLTPEQWKSHRSYSCQVTHEGSTVEKTVAPTECS
SEQ ID NO: 7
Polypeptide sequence of the secreted heavy chain of anti-PD-L1 (mut)/TGFβ Trap
EVQLLESGGGLVQPGGSLRLSCAASGFTFSMYMMMWVRQAPGKGLEWVSSIYPSGGIT
FYADSVKGRFTISRDNSKNTLYLQMNSLRAEDTAIYYCARIKLGTVTTVDYWGQGTLV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKTHTCPPCPAPE
LLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGAGGGGSGGGGSGGGGSGGGG
SGIPPHVQKSVNNDMIVTDNNGAVKFPQLCKFCDVRFSTCDNQKSCMSNCSITSICEKPQ
EVCVAVWRKNDENITLETVCHDPKLPYHDFILEDAASPKCIMKEKKKPGETFFMCSCSS
DECNDNIIFSEEYNTSNPD
SEQ ID NO: 8
Human TGFβRII Isoform A Precursor Polypeptide (NCBI RefSeq Accession No:
NP_001020018)
MGRGLLRGLWPLHIVLWTRIASTIPPHVQKSDVEMEAQKDEIICPSCNRTAHPLRHINND
MIVTDNNGAVKFPQLCKFCDVRFSTCDNQKSCMSNCSITSICEKPQEVCVAVWRKNDE
NITLETVCHDPKLPYHDFILEDAASPKCIMKEKKKPGETFFMCSCSSDECNDNIIFSEEYN
TSNPDLLLVIFQVTGISLLPPLGVAISVIIIFYCYRVNRQQKLSSTWETGKTRKLMEFSEHC
AIILEDDRSDISSTCANNINHNTELLPIELDTLVGKGRFAEVYKAKLKQNTSEQFETVAVK
IFPYEEYASWKTEKDIFSDINLKHENILQFLTAEERKTELGKQYWLITAFHAKGNLQEYL
TRHVISWEDLRKLGSSLARGIAHLHSDHTPCGRPKMPIVHRDLKSSNILVKNDLTCCLCD
FGLSLRLDPTLSVDDLANSGQVGTARYMAPEVLESRMNLENVESFKQTDVYSMALVL
WEMTSRCNAVGEVKDYEPPFGSKVREHPCVESMKDNVLRDRGRPEIPSFWLNHQGIQM
VCETLTECWDHDPEARLTAQCVAERFSELEHLDRLSGRSCSEEKIPEDGSLNTTK
SEQ ID NO: 9
Human TGFβRII Isoform B Precursor Polypeptide (NCBI RefSeq Accession No: NP_003233
MGRGLLRGLWPLHIVLWTRIASTIPPHVQKSVNNDMIVTDNNGAVKFPQLCKFCDVRFS
TCDNQKSCMSNCSITSICEKPQEVCVAVWRKNDENITLETVCHDPKLPYHDFILEDAASP
KCIMKEKKKPGETFFMCSCSSDECNDNIIFSEEYNTSNPDLLLVIFQVTGISLLPPLGVAIS
VIIIFYCYRVNRQQKLSSTWETGKTRKLMEFSEHCAIILEDDRSDISSTCANNINHNTELLP
IELDTLVGKGRFAEVYKAKLKQNTSEQFETVAVKIFPYEEYASWKTEKDIFSDINLKHEN
ILQFLTAEERKTELGKQYWLITAFHAKGNLQEYLTRHVISWEDLRKLGSSLARGIAHLHS
DHTPCGRPKMPIVHRDLKSSNILVKNDLTCCLCDFGLSLRLDPTLSVDDLANSGQVGTA
RYMAPEVLESRMNLENVESFKQTDVYSMALVLWEMTSRCNAVGEVKDYEPPFGSKVR
EHPCVESMKDNVLRDRGRPEIPSFWLNHQGIQMVCETLTECWDHDPEARLTAQCVAER
FSELEHLDRLSGRSCSEEKIPEDGSLNTTK
SEQ ID NO: 10
A Human TGFβRII Isoform B Extracellular Domain Polypeptide
IPPHVQKSVNNDMIVTDNNGAVKFPQLCKFCDVRFSTCDNQKSCMSNCSITSICEKPQEV
CVAVWRKNDENITLETVCHDPKLPYHDFILEDAASPKCIMKEKKKPGETFFMCSCSSDE
CNDNIIFSEEYNTSNPD
SEQ ID NO: 11
(Gly₄Ser)₄Gly linker
GGGGSGGGGSGGGGSGGGGSG
SEQ ID NO: 12
Polypeptide sequence of the secreted heavy chain variable region of anti-PD-L1 antibody
MPDL3289A
EVQLVESGGGLVQPGGSLRLSCAASGFTFSDSWIHWVRQAPGKGLEWVAWISPYGGST
YYADSVKGRFTISADTSKNTAYLQMNSLRAEDTAVYYCARRHWPGGFDYWGQGTLVT
VSS
SEQ ID NO: 13
Polypeptide sequence of the secreted light chain variable region of anti-PD-L1 antibody
MPDL3289A
DIQMTQSPSSLSASVGDRVTITCRASQDVSTAVAWYQQKPGKAPKLLIYSASFLYSGVPS
RFSGSGSGTDFTLTISSLQPEDFATYYCQQYLYHPATFGQGTKVEIKR
SEQ ID NO: 14
Polypeptide sequence of the secreted heavy chain variable region of anti-PD-L1 antibody
YW243.55S70
EVQLVESGGGLVQPGGSLRLSCAASGFTFSDSWIHWVRQAPGKGLEWVAWISPYGGST
YYADSVKGRFTISADTSKNTAYLQMNSLRAEDTAVYYCARRHWPGGFDYWGQGTLVT
VSA
SEQ ID NO: 50
A Truncated Human TGFβRII Isoform B Extracellular Domain Polypeptide
GAVKFPQLCKFCDVRFSTCDNQKSCMSNCSITSICEKPQEVCVAVWRKNDENITLETVC
HDPKLPYHDFILEDAASPKCIMKEKKKPGETFFMCSCSSDECNDNIIFSEEYNTSNPD
SEQ ID NO: 51
A Truncated Human TGFβRII Isoform B Extracellular Domain Polypeptide
VKFPQLCKFCDVRFSTCDNQKSCMSNCSITSICEKPQEVCVAVWRKNDENITLETVCHD
PKLPYHDFILEDAASPKCIMKEKKKPGETFFMCSCSSDECNDNIIFSEEYNTSNPD
SEQ ID NO: 52
A Truncated Human TGFβRII Isoform B Extracellular Domain Polypeptide
VTDNNGAVKFPQLCKFCDVRFSTCDNQKSCMSNCSITSICEKPQEVCVAVWRKNDENIT
LETVCHDPKLPYHDFILEDAASPKCIMKEKKKPGETFFMCSCSSDECNDNIIFSEEYNTSN
PD
SEQ ID NO: 53
A Truncated Human TGFβRII Isoform B Extracellular Domain Polypeptide
LCKFCDVRFSTCDNQKSCMSNCSITSICEKPQEVCVAVWRIKNDENITLETVCHDPKLPY
HDFILEDAASPKCIMKEKKKPGETFFMCSCSSDECNDNIIFSEEYNTSNPD
SEQ ID NO: 54
A Mutated Human TGFβRII Isoform B Extracellular Domain Polypeptide
VTDNAGAVKFPQLCKFCDVRFSTCDNQKSCMSNCSITSICEKPQEVCVAVWRKNDENIT
LETVCHDPKLPYHDFILEDAASPKCIMKEKKKPGETFFMCSCSSDECNDNIIFSEEYNTSN
PD
SEQ ID NO: 55
Polypeptide sequence of the heavy chain variable region of anti-PD-L1 antibody
QVQLQESGPGLVKPSQTLSLTCTVSGGSISNDYWTWIRQHPGKGLEYIGYISYTGSTYYN
PSLKSRVTISRDTSKNQFSLKLSSVTAADTAVYYCARSGGWLAPFDYWGRGTLVTVSS
SEQ ID NO: 56
Polypeptide sequence of the light chain variable region of anti-PD-L1 antibody
DIVMTQSPDSLAVSLGERATINCKSSQSLFYHSNQKHSLAWYQQKPGQPPKLLIYGAST
RESGVPDRFSGSGSGTDFTLTISSLQAEDVAVYYCQQYYGYPYTFGGGTKVEIK
SEQ ID NO: 57
Polypeptide sequence of the heavy chain variable region of anti-PD-L1 antibody
QVQLVQSGAEVKKPGASVKVSCKASGYTFTSYWMHWVRQAPGQGLEWMGRIGPNSG
FTSYNEKFKNRVTMTRDTSTSTVYMELSSLRSEDTAVYYCARGGSSYDYFDYWGQGTT
VTVSS
SEQ ID NO: 58
Polypeptide sequence of the light chain variable region of anti-PD-L1 antibody
DIVLIQSPASLAVSPGQRATITCRASESVSIHGTHLMHWYQQKPGQPPKLLIYAASNLES
GVPARFSGSGSGTDFTLTINPVEAEDTANYYCQQSFEDPLTFGQGTKLEIK
SEQ ID NO: 59
Polypeptide sequence of the heavy chain of anti-PD-L1 antibody
QVQLQESGPGLVKPSQTLSLTCTVSGGSISNDYWTWIRQHPGKGLEYIGYISYTGSTYYN
PSLKSRVTISRDTSKNQFSLKLSSVTAADTAVYYCARSGGWLAPFDYWGRGTLVTVSSA
STKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEAAGGPSVF
LFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEM
TKNQVSLICLVKGFYPSDIAVEWESNGQPENNYKTTPPVLDSDGSFFLYSRLTVDKSRW
QEGNVFSCSVMHEALHNHYTQKSLSLSLGK
SEQ ID NO: 60
Polypeptide sequence of the light chain of anti-PD-L1 antibody
DIVMTQSPDSLAVSLGERATINCKSSQSLFYHSNQKHSLAWYQQKPGQPPKLLIYGAST
RESGVPDRFSGSGSGTDFTLTISSLQAEDVAVYYCQQYYGYPYTFGGGTKVEIKRTVAA
PSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
SEQ ID NO: 61
Polypeptide sequence of the heavy chain of anti-PD-L1 antibody
QVQLVQSGAEVKKPGASVKVSCKASGYTFTSYWMHWVRQAPGQGLEWMGRIGPNSG
FTSYNEKFIKNRVTMTRDTSTSTVYMELSSLRSEDTAVYYCARGGSSYDYFDYWGQGTT
VTVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEAA
GGPSVFLFPPKPKDTLMISRTPEVTCVVVDVSQEDPEVQFNWYVDGVEVHNAKTKPRE
EQFNSTYRVVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGA
SEQ ID NO: 62
Polypeptide sequence of the light chain of anti-PD-L1 antibody
DIVLTQSPASLAVSPGQRATITCRASESVSIHGTHLMHWYQQKPGQPPKLLIYAASNLES
GVPARFSGSGSGTDFTLTINPVEAEDTANYYCQQSFEDPLTFGQGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

What is claimed is:

1. A method of treating a treatment naïve patient diagnosed with stage III non-small cell lung cancer (NSCLC), and at risk of developing a pathological disorder of the lung associated with concomitant chemotherapy and radiotherapy (cCRT), the method comprising a first step of administering to the patient a dose of at least 1200 mg of a protein comprising a first polypeptide and a second polypeptide, with concomitant cCRT, and a second step of administering at least 1200 mg of the protein without concomitant cCRT to the patient,
wherein the first polypeptide comprises: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ),
wherein the second polypeptide comprises at least a variable region of a light chain of an antibody that binds PD-L1, and
wherein the heavy chain of the first polypeptide and the light chain of the second polypeptide, when combined, form an antigen binding site that binds PD-L1.

2. The method of claim 1, wherein the method mitigates a pathological disorder of the lung associated with the cCRT at the first step.

3. The method of claim 2, wherein the pathological disorder is pneumonitis and/or pulmonary fibrosis.

4. The method of any one of claims 1-3, wherein the method increases the time-to-onset of metastasis and/or time to distant metastasis of the stage III NSCLC in the patient.

5. The method of any one of claims 1-4, wherein the first polypeptide comprises the amino acid sequence of SEQ ID NO: 3, and the second polypeptide comprises the amino acid sequence of SEQ ID NO: 1.

6. The method of any one of claims 1-5, wherein the dose is 1200 mg to 2400 mg.

7. The method of any one of claims 1-6, wherein the dose is 1800 mg to 2400 mg.

8. The method of any one of claims 1-7, wherein the dose is 1800 mg.

9. The method of any one of claims 1-7, wherein the dose is 2400 mg.

10. The method of any one of claims 1-6, wherein the dose is administered once every two weeks or once every three weeks.

11. The method of claim 10, wherein the dose is 1200 mg, administered once every two weeks.

12. The method of claim 10, wherein the dose is 2400 mg, administered once every three weeks.

13. The method of claim 10, wherein the dose is 2100 mg or 2400 mg, administered once every three weeks.

14. The method of any one of claims 1-13, wherein the stage III NSCLC exhibits squamous or non-squamous histology.

15. The method of any one of claims 1-14, wherein the stage III NSCLC exhibits PD-L1+ expression.

16. The method of any one of claims 1-14, wherein the stage III NSCLC does not exhibit PD-L1+ expression.

17. The method of any one of claims 1-16, wherein the patient has or does not have an EGFR sensitizing mutation.

18. The method of any one of claims 1-16, wherein the patient has or does not have an anaplastic lymphoma kinase (ALK) translocation.

19. The method of any one of claims 1-16, wherein the patient has or does not have ROS1 rearrangement.

20. The method of any one of claims 1-19, wherein the treatment results in a disease response or improved survival of the patient.

21. The method of claim 20, wherein the disease response is a complete response, a partial response, or a stable disease.

22. The method of claim 21, wherein the survival is progression-free survival (PFS).

23. The method of any one of claims 1-22, wherein the chemotherapy comprises administering cisplatin/etoposide, cisplatin/pemetrexed, and/or carboplatin/paclitaxel to the patient.

24. The method of any one of claims 1-23, wherein the chemotherapy comprises cisplatin/pemetrexed and the stage III NSCLC exhibits non-squamous histology.

25. The method of claim 23 or 24, wherein cisplatin is intravenously administered at a dose of about 50 mg/m²-80 mg/m².

26. The method of claim 23 or 24, wherein pemetrexed is intravenously administered at a dose of about 500 mg/m².

27. The method of claim 23, wherein etoposide is intravenously administered at a dose of about 50 mg/m².

28. The method of claim 23, wherein paclitaxel is intravenously administered at a dose of about 45 mg/m².

29. The method of claim 23, wherein carboplatin is intravenously administered based on AUC 2 over 30 minutes.

30. The method of any one of claims 1-29, wherein the radiotherapy comprises a dose of 60-74 Gy.

31. The method of claim 30, wherein the radiotherapy is administered on days 1-5 for 6-7 weeks during the first step.

32. The method of any one of claims 1-31, wherein the protein is administered by intravenous administration.

33. The method of claim 32, wherein the intravenous administration is performed with a prefilled bag, a prefilled pen, or a prefilled syringe comprising a formulation comprising the protein.

34. The method of claim 33, wherein the bag is connected to a channel comprising a tube and/or a needle.

35. The method of any one of claims 1-34, wherein the second step is initiated 1-42 days after completion of the first step.

36. The method of claim 35, wherein the second step is continued for 12-24 months.

37. A method of mitigating a pathological disorder associated with chemotherapy and radiotherapy (cCRT) in a treatment naïve patient diagnosed with stage III non-small cell lung cancer (NSCLC), the method comprising a first step of administering to the patient a dose of at least 1200 mg of a protein comprising a first polypeptide and a second polypeptide, with concomitant chemotherapy and radiotherapy (cCRT), and a second step of administering at least 1200 mg of the protein without concomitant cCRT to the patient,
wherein the first polypeptide comprises: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ),
wherein the second polypeptide comprises at least a variable region of a light chain of an antibody that hinds PD-L1, and
wherein the heavy chain of the first polypeptide and the light chain of the second polypeptide, when combined, form an antigen binding site that binds PD-L.

38. The method of claim 37, wherein the pathological disorder is pneumonitis and/or pulmonary fibrosis.

39. The method of claim 37 or 38, wherein the first polypeptide comprises the amino acid sequence of SEQ ID NO: 3, and the second polypeptide comprises the amino acid sequence of SEQ ID NO: 1.

40. The method of any one of claims 37-39, wherein the dose is 1200 mg to 2400 mg.

41. The method of any one of claims 37-40, wherein the dose is 1800 mg to 2400 mg.

42. The method of any one of claims 37-40, wherein the dose is 1200 mg.

43. The method of any one of claims 37-41, wherein the dose is 2400 mg.

44. The method of any one of claims 37-40, wherein the dose is administered once every two weeks or once every three weeks.

45. The method of claim 44, wherein the dose is 1200 mg, administered once every two weeks.

46. The method of claim 44, wherein the dose is 2400 mg, administered once every three weeks.

47. The method of claim 44, wherein the dose is 2100 mg or 2400 mg, administered once every three weeks.

48. The method of any one of claims 37-47, wherein the stage III NSCLC exhibits squamous or non-squamous histology.

49. The method of any one of claims 37-48, wherein the stage III NSCLC exhibits PD-L1+ expression.

50. The method of any one of claims 37-48, wherein the stage III NSCLC does not exhibit PD-L1+ expression.

51. The method of any one of claims 37-50, wherein the patient has or does not have an EGFR sensitizing mutation.

52. The method of any one of claims 37-50, wherein the patient has or does not have an anaplastic lymphoma kinase (ALK) translocation.

53. The method of any one of claims 37-50, wherein the patient has or does not have ROS1 rearrangement.

54. The method of any one of claims 37-53, wherein the treatment results in a disease response of the stage III NSCLC or improved survival of the patient.

55. The method of claim 54, wherein the disease response is a complete response, a partial response, or a stable disease.

56. The method of claim 55, wherein the survival is progression-free survival (PFS).

57. The method of any one of claims 37-56, wherein the chemotherapy comprises administering cisplatin/etoposide, cisplatin/pemetrexed, and/or carboplatin/paclitaxel to the patient.

58. The method of any one of claims 37-57, wherein the chemotherapy comprises cisplatin/pemetrexed and the stage III NSCLC exhibits non-squamous histology.

59. The method of claim 57 or 58, wherein cisplatin is intravenously administered at a dose of about 50 mg/m²-80 mg/m².

60. The method of claim 57 or 58, wherein pemetrexed is intravenously administered at a dose of about 500 mg/m².

61. The method of claim 57, wherein etoposide is intravenously administered at a dose of about 50 mg/m².

62. The method of claim 57, wherein paclitaxel is intravenously administered at a dose of about 45 mg/m².

63. The method of claim 57, wherein carboplatin is intravenously administered based on AUC 2 over 30 minutes.

64. The method of any one of claims 37-63, wherein the radiotherapy comprises a dose of 60-74 Gy.

65. The method of claim 64, wherein the radiotherapy is administered on days 1-5 for 6-7 weeks during the first step.

66. The method of any one of claims 37-65, wherein the protein is administered by intravenous administration.

67. The method of claim 66, wherein the intravenous administration is performed with a prefilled bag, a prefilled pen, or a prefilled syringe comprising a formulation comprising the protein.

68. The method of claim 67, wherein the bag is connected to a channel comprising a tube and/or a needle.

69. The method of any one of claims 37-68, wherein the second step is initiated 1-42 days after completion of the first step.

70. The method of claim 69, wherein the second step is continued for 12-24 months.

71. The method of any one of claims 1-70, wherein the stage III non-small cell lunch cancer (NSCLC) is unresectable.

72. The method of any one of claims 1-22 and 37-56, wherein the chemotherapy is a platinum-based chemotherapy.

73. An anti-PD-L1/TGFβ Trap protein comprising a first polypeptide and a second polypeptide for use in a method of treating a treatment naïve patient diagnosed with stage III non-small cell lung cancer (NSCLC), and at risk of developing a pathological disorder of the lung associated with concomitant chemotherapy and radiotherapy (cCRT), the method comprising a first step of administering to the patient a dose of at least 1200 mg of the protein with concomitant cCRT, and a second step of administering at least 1200 mg of the protein without concomitant cCRT to the patient,
wherein the first polypeptide comprises: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGF),
wherein the second polypeptide comprises at least a variable region of a light chain of an antibody that binds PD-L1, and
wherein the heavy chain of the first polypeptide and the light chain of the second polypeptide, when combined, form an antigen binding site that binds PD-L1.

74. An anti-PD-L1/TGFβ Trap protein comprising a first polypeptide and a second polypeptide for use in a method of mitigating a pathological disorder associated with chemotherapy and radiotherapy (cCRT) in a treatment naïve patient diagnosed with stage III non-small cell lung cancer (NSCLC), the method comprising a first step of administering to the patient a dose of at least 1200 mg of the protein with concomitant chemotherapy and radiotherapy (cCRT), and a second step of administering at least 1200 mg of the protein without concomitant cCRT to the patient,
wherein the first polypeptide comprises: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ),
wherein the second polypeptide comprises at least a variable region of a light chain of an antibody that binds PD-L1, and
wherein the heavy chain of the first polypeptide and the light chain of the second polypeptide, when combined, form an antigen binding site that binds PD-L1.

75. The anti-PD-L1/TGFβ Trap protein for use of claim 73 or 74, wherein the method mitigates a pathological disorder of the lung associated with the cCRT at the first step.

76. The anti-PD-L1/TGFβ Trap protein for use of claim 75, wherein the pathological disorder is pneumonitis and/or pulmonary fibrosis.

77. The anti-PD-L1/TGFβ Trap protein for use of any one of claims 73-76, wherein the method increases the time-to-onset of metastasis and/or time to distant metastasis of the stage III NSCLC in the patient.

78. The anti-PD-L1/TGFβ Trap protein for use of any one of claims 73-77, wherein the first polypeptide comprises the amino acid sequence of SEQ ID NO: 3, and the second polypeptide comprises the amino acid sequence of SEQ ID NO: 1.

79. The anti-PD-L1/TGFβ Trap protein for use of any one of claims 73-78, wherein the dose is 1200 mg to 2400 mg.

80. The anti-PD-L1/TGFβ Trap protein for use of any one of claims 73-79, wherein the dose is 1800 mg to 2400 mg.

81. The anti-PD-L1/TGFβ Trap protein for use of any one of claims 73-79, wherein the dose is 1200 mg.

82. The anti-PD-L1/TGFβ Trap protein for use of any one of claims 73-80, wherein the dose is 2400 mg.

83. The anti-PD-L1/TGFβ Trap protein for use of any one of claims 73-79, wherein the dose is administered once every two weeks or once every three weeks.

84. The anti-PD-L1/TGFβ Trap protein for use of claim 83, wherein the dose is 1200 mg, administered once every two weeks.

85. The anti-PD-L1/TGFβ Trap protein for use of claim 83, wherein the dose is 2400 mg, administered once every three weeks.

86. The anti-PD-L1/TGFβ Trap protein for use of claim 79, wherein the dose is 2100 mg or 2400 mg, administered once every three weeks.

87. The anti-PD-L1/TGFβ Trap protein for use of any one of claims 73-86, wherein the stage III NSCLC exhibits squamous or non-squamous histology.

88. The anti-PD-L/TGFβ Trap protein for use of any one of claims 73-87, wherein the stage III NSCLC exhibits PD-L1+ expression.

89. The anti-PD-L1/TGFβ Trap protein for use of any one of claims 73-87, wherein the stage III NSCLC does not exhibit PD-L1+ expression.

90. The anti-PD-L1/TGFβ Trap protein for use of any one of claims 73-89, wherein the patient has or does not have an EGFR sensitizing mutation.

91. The anti-PD-L1/TGFβ Trap protein for use of any one of claims 73-89, wherein the patient has or does not have an anaplastic lymphoma kinase (ALK) translocation.

92. The anti-PD-L1/TGFβ Trap protein for use of any one of claims 73-89, wherein the patient has or does not have ROS1 rearrangement.

93. The anti-PD-L1/TGFβ Trap protein for use of any one of claims 73-92, wherein the treatment results in a disease response or improved survival of the patient.

94. The anti-PD-L1/TGFβ Trap protein for use of claim 93, wherein the disease response is a complete response, a partial response, or a stable disease.

95. The anti-PD-L1/TGFβ Trap protein for use of claim 93, wherein the survival is progression-free survival (PFS).

96. The anti-PD-L1/TGFβ Trap protein for use of any one of claims 73-95, wherein the chemotherapy comprises administering cisplatin/etoposide, cisplatin/pemetrexed, and/or carboplatin/paclitaxel to the patient.

97. The anti-PD-L1/TGFβ Trap protein for use of any one of claims 73-95, wherein the chemotherapy comprises cisplatin/pemetrexed and the stage III NSCLC exhibits non-squamous histology.

98. The anti-PD-L1/TGFβ Trap protein for use of claim 96 or 97, wherein cisplatin is intravenously administered at a dose of about 50 mg/m²-80 mg/m².

99. The anti-PD-L1/TGFβ Trap protein for use of claim 96 or 97, wherein pemetrexed is intravenously administered at a dose of about 500 mg/m².

100. The anti-PD-L1/TGFβ Trap protein for use of claim 96, wherein etoposide is intravenously administered at a dose of about 50 mg/m².

101. The anti-PD-L1/TGFβ Trap protein for use of claim 96, wherein paclitaxel is intravenously administered at a dose of about 45 mg/m².

102. The anti-PD-L1/TGFβ Trap protein for use of claim 96, wherein carboplatin is intravenously administered based on AUC 2 over 30 minutes.

103. The anti-PD-L1/TGFβ Trap protein for use of any one of claims 73-102, wherein the radiotherapy comprises a dose of 60-74 Gy.

104. The anti-PD-L1/TGFβ Trap protein for use of claim 103, wherein the radiotherapy is administered on days 1-5 for 6-7 weeks during the first step.

105. The anti-PD-L1/TGFβ Trap protein for use of any one of claims 73-104, wherein the protein is administered by intravenous administration.

106. The anti-PD-L1/TGFβ Trap protein for use of claim 105, wherein the intravenous administration is performed with a prefilled bag, a prefilled pen, or a prefilled syringe comprising a formulation comprising the protein.

107. The anti-PD-L1/TGFβ Trap protein for use of claim 106, wherein the bag is connected to a channel comprising a tube and/or a needle.

108. The anti-PD-L1/TGFβ Trap protein for use of any one of claims 73-107, wherein the second step is initiated 1-42 days after completion of the first step.

109. The anti-PD-L1/TGFβ Trap protein for use of claim 108, wherein the second step is continued for 12-24 months.


SEQUENCE LISTING (62 sequences)

SEQ ID NO: 1 - protein, 216 aa, Artificial Sequence
  Description of Artificial Sequence Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 2 - protein, 450 aa, Artificial Sequence
  Description of Artificial Sequence Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 607 aa, Artificial Sequence
  Description of Artificial Sequence Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 4 - DNA, 711 bp, Artificial Sequence
  Description of Artificial Sequence Synthetic polynucleotide
atgagggccctgctggctagactgctgctgtgcgtgctggtcgtgtccgacagcaagggccagtccgccctgacccagcctgcctccgtgtctggctcccctggccagtccatcaccatcagctgcaccggcacctccagcgacgtgggcggctacaactacgtgtcctggtatcagcagcaccccggcaaggcccccaagctgatgatctacgacgtgtccaaccggccctccggcgtgtccaacagattctccggctccaagtccggcaacaccgcctccctgaccatcagcggactgcaggcagaggacgaggccgactactactgctcctcctacacctcctccagcaccagagtgttcggcaccggcacaaaagtgaccgtgctgggccagcccaaggccaacccaaccgtgacactgttccccccatcctccgaggaactgcaggccaacaaggccaccctggtctgcctgatctcagatttctatccaggcgccgtgaccgtggcctggaaggctgatggctccccagtgaaggccggcgtggaaaccaccaagccctccaagcagtccaacaacaaatacgccgcctcctcctacctgtccctgacccccgagcagtggaagtcccaccggtcctacagctgccaggtcacacacgagggctccaccgtggaaaagaccgtcgcccccaccgagtgctcatga

SEQ ID NO: 5 - DNA, 1887 bp, Artificial Sequence
  Description of Artificial Sequence Synthetic polynucleotide
atggaaacagacaccctgctgctgtgggtgctgctgctgtgggtgcccggctccacaggcgaggtgcagctgctggaatccggcggaggactggtgcagcctggcggctccctgagactgtcttgcgccgcctccggcttcaccttctccagctacatcatgatgtgggtgcgacaggcccctggcaagggcctggaatgggtgtcctccatctacccctccggcggcatcaccttctacgccgacaccgtgaagggccggttcaccatctcccgggacaactccaagaacaccctgtacctgcagatgaactccctgcgggccgaggacaccgccgtgtactactgcgcccggatcaagctgggcaccgtgaccaccgtggactactggggccagggcaccctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtgctggcggcggaggaagcggaggaggtggcagcggtggcggtggctccggcggaggtggctccggaatccctccccacgtgcagaagtccgtgaacaacgacatgatcgtgaccgacaacaacggcgccgtgaagttccctcagctgtgcaagttctgcgacgtgaggttcagcacctgcgacaaccagaagtcctgcatgagcaactgcagcatcacaagcatctgcgagaagccccaggaggtgtgtgtggccgtgtggaggaagaacgacgaaaacatcaccctcgagaccgtgtgccatgaccccaagctgccctaccacgacttcatcctggaagacgccgcctcccccaagtgcatcatgaaggagaagaagaagcccggcgagaccttcttcatgtgcagctgcagcagcgacgagtgcaatgacaacatcatctttagcgaggagtacaacaccagcaaccccgactgataa

SEQ ID NO: 6 - protein, 216 aa, Artificial Sequence
  Description of Artificial Sequence Synthetic polypeptide
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 7 - protein, 607 aa, Artificial Sequence
  Description of Artificial Sequence Synthetic polypeptide
EVQLLESGGGLVQPGGSLRLSCAASGFTFSMYMMMWVRQAPGKGLEWVSSIYPSGGITFYADSVKGRFTISRDNSKNTLYLQMNSLRAEDTAIYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 8 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 9 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 10 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 11 - protein, 21 aa, Artificial Sequence
  Description of Artificial Sequence Synthetic peptide
GGGGSGGGGSGGGGSGGGGSG

SEQ ID NO: 12 - protein, 118 aa, Artificial Sequence [sequence omitted]

SEQ ID NO: 13 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 14 - protein, 118 aa, Artificial Sequence
  Description of Artificial Sequence Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 15 - protein, 4 aa, Artificial Sequence
  Description of Artificial Sequence Synthetic peptide
QFNS

SEQ ID NO: 16 - protein, 4 aa, Artificial Sequence
  Description of Artificial Sequence Synthetic peptide
QAQS

SEQ ID NO: 17 - protein, 6 aa, Artificial Sequence
  Description of Artificial Sequence Synthetic peptide
PKSCDK

SEQ ID NO: 18 - protein, 6 aa, Artificial Sequence
  Description of Artificial Sequence Synthetic peptide
PKSSDK

SEQ ID NO: 19 - protein, 4 aa, Artificial Sequence
  Description of Artificial Sequence Synthetic peptide
LSLS

SEQ ID NO: 20 - protein, 4 aa, Artificial Sequence
  Description of Artificial Sequence Synthetic peptide
ATAT

SEQ ID NO: 21 - protein, 5 aa, Artificial Sequence
  Description of Artificial Sequence Synthetic peptide
  MOD_RES (1)..(1): Lys, Arg, Thr, Gln, Gly, Ala, Trp, Met, Ile or Ser
  MOD_RES (3)..(3): Val, Arg, Lys, Leu, Met or Ile
  MOD_RES (5)..(5): His, Thr, Asn, Gln, Ala, Val, Tyr, Trp, Phe or Met
XYXMX

SEQ ID NO: 22 - protein, 17 aa, Artificial Sequence
  Description of Artificial Sequence Synthetic peptide
  MOD_RES (8)..(8): Phe or Ile
  MOD_RES (14)..(14): Ser or Thr
SIYPSGGXTFYADXVKG

SEQ ID NO: 23 - protein, 11 aa, Artificial Sequence
  Description of Artificial Sequence Synthetic peptide
  MOD_RES (10)..(10): Glu or Asp
IKLGTVTTVXY

SEQ ID NO: 24 - protein, 30 aa, Artificial Sequence
  Description of Artificial Sequence Synthetic polypeptide
EVQLLESGGGLVQPGGSLRLSCAASGFTFS

SEQ ID NO: 25 - protein, 14 aa, Artificial Sequence
  Description of Artificial Sequence Synthetic peptide
WVRQAPGKGLEWVS

SEQ ID NO: 26 - protein, 32 aa, Artificial Sequence
  Description of Artificial Sequence Synthetic polypeptide
RFTISRDNSKNTLYLQMNSLRAEDTAVYYCAR

SEQ ID NO: 27 - protein, 11 aa, Artificial Sequence
  Description of Artificial Sequence Synthetic peptide
WGQGTLVTVSS

SEQ ID NO: 28 - protein, 14 aa, Artificial Sequence
  Description of Artificial Sequence Synthetic peptide
  MOD_RES (4)..(4): Asn or Ser
  MOD_RES (5)..(5): Thr, Arg or Ser
  MOD_RES (9)..(9): Ala or Gly
TGTXXDVGXYNYVS

SEQ ID NO: 29 - protein, 7 aa, Artificial Sequence
  Description of Artificial Sequence Synthetic peptide
  MOD_RES (1)..(1): Glu or Asp
  MOD_RES (3)..(3): Ile, Asn or Ser
  MOD_RES (4)..(4): Asp, His or Asn
XVXXRPS

SEQ ID NO: 30 - protein, 10 aa, Artificial Sequence
  Description of Artificial Sequence Synthetic peptide
  MOD_RES (3)..(3): Phe or Tyr
  MOD_RES (5)..(5): Asn or Ser
  MOD_RES (6)..(6): Arg, Thr or Ser
  MOD_RES (7)..(7): Gly or Ser
  MOD_RES (8)..(8): Ile or Thr
SSXTXXXXRV

SEQ ID NO: 31 - protein, 22 aa, Artificial Sequence
  Description of Artificial Sequence Synthetic peptide
QSALTQPASVSGSPGQSITISC

SEQ ID NO: 32 - protein, 15 aa, Artificial Sequence
  Description of Artificial Sequence Synthetic peptide
WYQQHPGKAPKLMIY

SEQ ID NO: 33 - protein, 32 aa, Artificial Sequence
  Description of Artificial Sequence Synthetic polypeptide
GVSNRFSGSKSGNTASLTISGLQAEDEADYYC

SEQ ID NO: 34 - protein, 10 aa, Artificial Sequence
  Description of Artificial Sequence Synthetic peptide
FGTGTKVTVL

SEQ ID NO: 35 - protein, 5 aa, Artificial Sequence
  Description of Artificial Sequence Synthetic peptide
SYIMM

SEQ ID NO: 36 - protein, 17 aa, Artificial Sequence
  Description of Artificial Sequence Synthetic peptide
SIYPSGGITFYADTVKG

SEQ ID NO: 37 - protein, 11 aa, Artificial Sequence
  Description of Artificial Sequence Synthetic peptide
IKLGTVTTVDY

SEQ ID NO: 38 - protein, 14 aa, Artificial Sequence
  Description of Artificial Sequence Synthetic peptide
TGTSSDVGGYNYVS

SEQ ID NO: 39 - protein, 7 aa, Artificial Sequence
  Description of Artificial Sequence Synthetic peptide
DVSNRPS

SEQ ID NO: 40 - protein, 10 aa, Artificial Sequence
  Description of Artificial Sequence Synthetic peptide
SSYTSSSTRV

SEQ ID NO: 41 - protein, 5 aa, Artificial Sequence
  Description of Artificial Sequence Synthetic peptide
MYMMM

SEQ ID NO: 42 - protein, 17 aa, Artificial Sequence
  Description of Artificial Sequence Synthetic peptide
SIYPSGGITFYADSVKG

SEQ ID NO: 43 - protein, 14 aa, Artificial Sequence
  Description of Artificial Sequence Synthetic peptide
TGTSSDVGAYNYVS

SEQ ID NO: 44 - protein, 119 aa, Artificial Sequence
  Description of Artificial Sequence Synthetic polypeptide
EVQLLESGGGLVQPGGSLRLSCAASGFTFSSYIMMVWRQAPGKGLEWVSSIYPSGGITFYADWKGRFTISRDNSKNTLYLQMNSLRAEDTAVYYCARIKLGTVTTVDYWGQGTLVTVSS

SEQ ID NO: 45 - protein, 110 aa, Artificial Sequence
  Description of Artificial Sequence Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 46 - protein, 120 aa, Artificial Sequence
  Description of Artificial Sequence Synthetic polypeptide
EVQLLESGGGLVQPGGSLRLSCAASGFTFSMYMMMWVRQAPGKGLEVWSSIYPSGGITFYADSVKGRFTISRDNSKNTLYLQMNSLRAEDTAIYYCARIKLGTVTTVDYWGQGTLVTVSS

SEQ ID NO: 47 - protein, 110 aa, Artificial Sequence
  Description of Artificial Sequence Synthetic polypeptide
QSALTQPASVSGSPGQSITISCTGTSSDVGAYNYVSWYQQHPGKAPKLMIYDVSNRPSGVSNRFSGSKSGNTASLTISGLQAEDEADYYCSSYTSSSTRVFGTGTKVTVL

SEQ ID NO: 48 - DNA, 1407 bp, Artificial Sequence
  Description of Artificial Sequence Synthetic polynucleotide from human Fab library
atggagttgcctgttaggctgttggtgctgatgttctggattcctgctagctccagcgaggtgcagctgctggaatccggcggaggactggtgcagcctggcggctccctgagactgtcttgcgccgcctccggcttcaccttctccagctacatcatgatgtgggtgcgacaggcccctggcaagggcctggaatgggtgtcctccatctacccctccggcggcatcaccttctacgccgacaccgtgaagggccggttcaccatctcccgggacaactccaagaacaccctgtacctgcagatgaactccctgcgggccgaggacaccgccgtgtactactgcgcccggatcaagctgggcaccgtgaccaccgtggactactggggccagggcaccctggtgacagtgtcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 49 - DNA, 705 bp, Artificial Sequence
  Description of Artificial Sequence Synthetic polynucleotide from human Fab library
atggagttgcctgttaggctgttggtgctgatgttctggattcctgcttccttaagccagtccgccctgacccagcctgcctccgtgtctggctcccctggccagtccatcaccatcagctgcaccggcacctccagcgacgtgggcggctacaactacgtgtcctggtatcagcagcaccccggcaaggcccccaagctgatgatctacgacgtgtccaaccggccctccggcgtgtccaacagattctccggctccaagtccggcaacaccgcctccctgaccatcagcggactgcaggcagaggacgaggccgactactactgctcctcctacacctcctccagcaccagagtgttcggcaccggcacaaaagtgaccgtgctgggccagcccaaggccaacccaaccgtgacactgttccccccatcctccgaggaactgcaggccaacaaggccaccctggtctgcctgatctcagatttctatccaggcgccgtgaccgtggcctggaaggctgatggctccccagtgaaggccggcgtggaaaccaccaagccctccaagcagtccaacaacaaatacgccgcctcctcctacctgtccctgacccccgagcagtggaagtcccaccggtcctacagctgccaggtcacacacgagggctccaccgtggaaaagaccgtcgcccccaccgagtgctca

SEQ ID NO: 50 - protein, 117 aa, Homo sapiens
GAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 51 - protein, 115 aa, Homo sapiens
VKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 52 - protein, 122 aa, Homo sapiens [sequence omitted]

SEQ ID NO: 53 - protein, 110 aa, Homo sapiens
LCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 54 - protein, 122 aa, Artificial Sequence
  Description of Artificial Sequence Synthetic polypeptide
VTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 55 - protein, 118 aa, Artificial Sequence
  Description of Artificial Sequence Synthetic polypeptide
QVQLQESGPGLVKPSQTLSLTCTVSGGSISNDYWTWIRQHPGKGLEYIGYISYTGSTYYNPSLKSRVTISRDTSKNQFSLKLSSVTAADTAVYYCARSGGWLAPFDYWGRGTLVTVSS

SEQ ID NO: 56 - protein, 113 aa, Artificial Sequence
  Description of Artificial Sequence Synthetic polypeptide
DIVMTQSPDSLAVSLGERATINCKSSQSLFYHSNQKHSLAWYQQKPGQPPKLLIYGASTRESGVPDRFSGSGSGTDFTLTISSLQAEDVAVYYCQQYYGYPYTFGGGTKVEIK

SEQ ID NO: 57 - protein, 119 aa, Artificial Sequence
  Description of Artificial Sequence Synthetic polypeptide
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSS

SEQ ID NO: 58 - protein, 111 aa, Artificial Sequence
  Description of Artificial Sequence Synthetic polypeptide
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIK

SEQ ID NO: 59 - protein, 445 aa, Artificial Sequence
  Description of Artificial Sequence Synthetic polypeptide
QVQLQESGPGLVKPSQTLSLTCTVSGGSISNDYWTWIRQHPGKGLEYIGYISYTGSTYYNPSLKSRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 220 aa, Artificial Sequence
  Description of Artificial Sequence Synthetic polypeptide
DIVMTQSPDSLAVSLGERATINCKSSQSLFYHSNQKHSLAWYQQKPGQPPKLLIYGASTRESGVPDRFSGSGSGTDFTLTISSLQAEDVAVYYCQQYYGYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 446 aa, Artificial Sequence
  Description of Artificial Sequence Synthetic polypeptide
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGA

SEQ ID NO: 62 - protein, 218 aa, Artificial Sequence
  Description of Artificial Sequence Synthetic polypeptide
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC